(19) United States
(12) Patent Application Publication
(10) Pub. No.: US 2023/0140838 A9
(43) Pub. Date: May 4, 2023

(54) NOVEL ACTIVITY-BASED PROBES FOR NEUTROPHIL ELASTASE AND THEIR USE

(71) Applicant: Takeda Pharmaceutical Company Limited, Osaka (JP)

(72) Inventors: Laura EDGINGTON-MITCHELL, Clayton (AU); Bethany M. ANDERSON, Clayton (AU); Daniel P. POOLE, Clayton (AU); Luigi AURELIO, Clayton (AU); Paulina KASPERKIEWICZ, Wroclaw (PL); Marcin DRAG, Wroclaw (PL); Nigel BUNNETT, Clayton (AU)

(73) Assignee: Takeda Pharmaceutical Company Limited, Osaka (JP)

(21) Appl. No.: 17/416,053

(22) PCT Filed: Dec. 20, 2019
(86) PCT No.: PCT/JP2019/050223

§ 371 Date: Jun. 18, 2021

(30) Foreign Application Priority Data
Dec. 20, 2018 (AU) 2018904873

(51) Int. Cl. G01N 33/573; G01N 33/58; G01N 33/559; C09B 23/01; C09K 11/06; G01N 21/64

(52) U.S. Cl. CPC G01N 33/573; G01N 33/582; G01N 33/559; C09B 23/0091; C09K 11/06; G01N 21/6428; G01N 2333/966; G01N 2474/10; G01N 2800/06; G01N 2800/26; C09K 2211/1007; C09K 2211/1014; C09K 2211/1022; G01N 2021/6439

(57) ABSTRACT

The present invention relates to compounds of formula I, wherein D is a detectable moiety, or salts thereof, which can be used as activity-based probes for neutrophil elastase, as well as to methods for detecting neutrophil elastase (NE) activity in a tissue sample lysate, and related diagnostic methods using compounds of formula I.

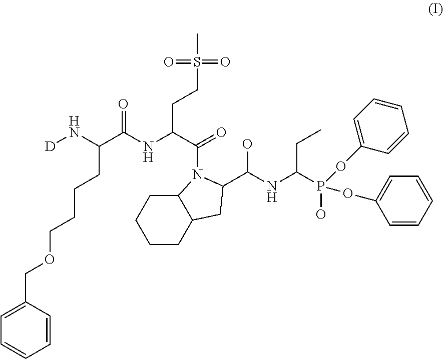

### TECHNICAL FIELD

The present invention relates to compounds that can be used as activity-based probes and/or inhibitors for neutrophil elastase, methods of detecting neutrophil elastase activity, and related diagnostic methods.

### BACKGROUND ART

According to Lechtenberg et al., ACS Chem. Biol. (2015), “proteases are central mediators of a large variety of physiological processes. Proteolytic cleavage events are at the basis of protein degradation, enzyme activation, and protein maturation and regulate a wide range of pathways from cell death, migration and proliferation, inflammation and immune response, to blood coagulation (Rawlings and Salvesen (2012)). Aberrant proteolysis on the other hand is frequently linked to serious disorders. Furthermore, proteases are usually expressed in the cell or secreted as inactive zymogens that need activation via processes like proteolytic cleavage or dimerization. Activation of proteases underlies tight temporal and spatial regulation, and thus generally protease location is not an ideal marker for protease function. Instead, spatial-temporal location of the active form of a given protease is necessary for understanding its function. For this purpose, activity-based probes have been developed for a variety of proteases (Deu et al., Nat. Struct. Mol. Biol. (2012)). These probes are designed like active site-reacting protease inhibitors to specifically label an active protease and are thus powerful tools for research and diagnostics. Furthermore, these probes additionally pave the way for the development of potent inhibitors for select proteases for potential therapeutic use (Deu et al., Nat. Struct. Mol. Biol. (2012)).” Lechtenberg et al. go on to describe neutrophil elastase (NE) as “a prime example for a desirable yet difficult target for activity-based probe design.”

NE is a serine protease, that is a protease using a serine residue in its active site as the nucleophilic amino acid for proteolysis, found within azurophilic granules of neutrophils (Korkmaz et al. Pharmacol Rev (2010)). During infection, active NE contributes to killing of intracellular pathogens by cleaving microbial proteins (Korkmaz et al. Pharmacol Rev (2010); Kobayashi et al. Arch Immunol Ther Exp (2005)). Mice lacking NE are more susceptible to bacterial and fungal infections (Reeves et al. Nature (2002). NE also mediates inflammation by processing cytokines, chemokines and growth factors (Korkmaz et al. Pharmacol Rev (2010)). Furthermore, NE cleaves the extracellular N-termini of protease-activated receptors (PARs), a family of Gprotein-coupled receptors (GPCRs), to initiate cellular signaling events that lead to inflammation and pain (Jimenez-Vargas et al. Proc Natl Acad Sci (2018); Lieu et al. Brit J Pharmacol (2016); Zhao et al. J Biol Chem (2015)). NE can also facilitate tissue destruction through cleavage of extracellular matrix components. It is becoming increasingly appreciated that NE activity is increased in cancers of the breast, prostate, colon/rectum, and lung (Lerman & Hammes. Steroids (2018)). NE is also involved in the development of chronic obstructive pulmonary diseases (Demkow & Overveld Eur J Med Res (2010)), and lung infections (Polverino et al. Chest (2017)), likely including Legionella infections (Narita et al. Nihon Kokyuki Gakkai Zasshi (2007)).

Aside from its roles in infection and cancer, NE has recently been implicated in the pathogenesis of inflammatory bowel diseases (IBD), which are characterized by chronic and relapsing inflammation in the gastrointestinal tract (Edgington-Mitchell. Am J Physiol Gastrointest Liver Physiol (2015)). IBD comprises ulcerative colitis (UC) and Crohn's disease (CD), both of which are associated with diarrhea, rectal bleeding, increased urgency and pain. Mice lacking one copy of NE and a related neutrophil serine protease, proteinase 3 (PR3), exhibit improved symptoms in mouse models of colitis (Motta et al. Gastroenterol (2011)). Enforced expression of elafin, an endogenous serine protease inhibitor, either by intracolonic administration of adenoviral vectors or introduction of elafin-expressing lactic acid bacteria, resulted in attenuation of symptoms in mouse models of colitis (Motta et al. Gastroenterol (2011)). Treatment with a NE-selective inhibitor also reduced colitis symptoms (Morohoshi et al. J Gastroenterol (2006)). Colonic mucosal biopsies from patients with IBD exhibit elevated NE expression compared to healthy controls at both mRNA and protein levels (Kuno et al. J Gastroenterol (2002); Uchiyama et al. Am J Physiol Gastrointest Liver Physiol (2012)).

Because NE is expressed as an inactive zymogen and can be tightly controlled by endogenous inhibitors once activated, measures of mRNA or total protein expression rarely reflect the pool of active functional enzyme (Edgington et al. Curr Op Chem Biol (2011)). Thus, tools to measure the specific activity of NE are required to more accurately determine its involvement in pathologies.

Commercially available chromogenic and fluorogenic substrate probes, including AAPV-p-nitroanilide and BODIPY-FL-elastin, respectively, indicated an increase in elastase-like activity in biopsies from UC and CD patients and in mouse models of IBD (Gecse K et al. Gut (2008); Morohoshi et al. J Gastroenterol (2006); Motta et al. Sci Trans Med (2012); Motta et al. Gastroenterol (2011)). However, these probes not only lack specificity, but can be cleaved by multiple proteases which are present in tissues and tissue samples (Edgington et al. Curr Op Chem Biol (2011); Edgington-Mitchell. Am J Physiol Gastrointest Liver Physiol (2015)).

A fluorescent activity-based probe (ABP) for NE, Cy5-V-DPP, was previously used to track NE activation during colitis (Edgington-Mitchell et al. Bioorganic Med Chem Lett (2017)). This probe contained a sulfonated cyanine 5 (sulfoCy5) fluorophore and a P1 valine residue coupled to a diphenylphosphonate electrophile (DPP; ‘warhead’) that reacts with the active site serine of active NE in a covalent, irreversible manner. While Cy5-V-DPP efficiently labeled recombinant NE and endogenous NE in purified cells with high expression (e.g., bone marrow), lack of sensitivity led to little success in detecting NE activity in colitis tissues.

Thus, there is a need for activity-based probes for NE allowing detection of NE activity in more complex samples such as tissue lysates, e.g. probes exhibiting resistance to cleavage by other proteases and/or high enough sensitivity to permit labeling of NE, and thereby detection of NE activity, in tissue lysates. Similarly, there is a need for methods employing these activity-based probes in order to detect NE activity in tissue lysates, to inhibit NE in tissue lysates, and/or to diagnose a subject with pathologies in which NE activity has a role by testing tissue lysates.

The present inventors now found that activity-based probe compounds as described below bearing a detectable element, the recognition sequence Nle(O-Bzl)-Met(O)₂-Oic-Abu, and a DPP warhead, can be used as activity-based probes for detection of NE activity in tissue lysates. Compounds of this structure, such as the PK101 probe (exhibiting a biotin tag, a PEG linker, the recognition sequence Nle(O-Bzl)-Met(O)₂-Oic-Abu and a DPP warhead), and probes of the PK10X series (exhibiting different tags, a PEG linker, the recognition sequence Nle(O-Bzl)-Met(O)₂-Oic-Abu and a DPP warhead) have so far only been described as possessing efficacy and specificity for NE in purified cells (Kasperkiewicz et al. J Am Chem Soc (2017); Kasperkiewicz et al. FEBS (2017); Kasperkiewicz et al. Proc Natl Acad Sci (2014); Lechtenberg et al. ACS Chem Biol (2015)), which represent a much less challenging sample type.

### SUMMARY OF INVENTION

It is an object of certain embodiments of the present invention to provide methods of detecting neutrophil elastase activity in tissue sample lysates.

It is an object of certain embodiments of the present invention to provide in vitro methods of diagnosing a disease associated with (increased) neutrophil elastase activity.

It is an object of certain embodiments of the present invention to provide in vitro methods of diagnosing a disease selected from the group consisting of a celiac disease, a gastrointestinal motility disorder, pain, itch, a skin disorder, diet-induced obesity, a metabolic disorder, asthma, rheumatoid arthritis, periodontitis, an inflammatory GI disorder, a functional GI disorder, a cancer, a fibrotic disease, metabolic dysfunction, a neurological disease, a chronic obstructive pulmonary disease (COPD), and an infection.

It is an object of certain embodiments of the present invention to provide in vitro methods of diagnosing a disease selected from the group of an inflammatory bowel disease, an infection, a chronic obstructive pulmonary disease, and a cancer in a subject.

It is an object of certain embodiments of the present invention to provide in vitro methods of inhibiting neutrophil elastase.

It is an object of certain embodiments of the present invention to provide activity-based probe compounds that allow detection of neutrophil elastase activity with high potency for neutrophil elastase, such as improved potency for neutrophil elastase as compared to the activity-based probe Cy5-V-DPP, e.g., in tissue lysates.

It is an object of certain embodiments of the present invention to provide inhibitors of neutrophil elastase.

It is an object of certain embodiments of the present invention to provide compounds that can be used in the diagnosis of a disease associated with neutrophil elastase activity.

It is an object of certain embodiments of the present invention to provide compounds that can be used in the diagnosis of a disease selected from the group consisting of a celiac disease, a gastrointestinal motility disorder, pain, itch, a skin disorder, diet-induced obesity, a metabolic disorder, asthma, rheumatoid arthritis, periodontitis, an inflammatory GI disorder, a functional GI disorder, a cancer, a fibrotic disease, metabolic dysfunction, a neurological disease, a chronic obstructive pulmonary disease (COPD), and an infection.

It is an object of certain embodiments of the present invention to provide compounds that can be used in the diagnosis of a disease selected from the group consisting of an inflammatory bowel disease, an infection, a chronic obstructive pulmonary disease, and a cancer.

The above objects are to be understood to also relate to the respective methods as well as to compounds/compositions for use in the respective method.

In certain embodiments, the present invention is directed to a method of detecting neutrophil elastase (NE) activity in a tissue sample lysate, comprising

(1) preparing the lysate from a tissue sample obtained from a subject,

(2) contacting the lysate with a compound of formula I

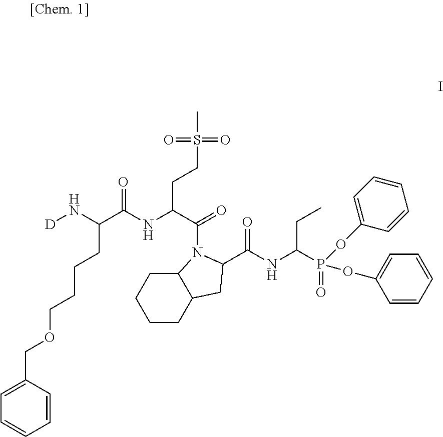

or a salt thereof,

wherein D is a detectable element,

(3) subsequently subjecting at least an aliquot of the lysate of step (2) to gel electrophoresis; and thereafter

(4) measuring a detectable signal.

In certain embodiments, the present invention is directed to a method of diagnosing a disease associated with NE activity in a subject comprising

(1) preparing a lysate from a tissue sample obtained from the subject,

(2) contacting the lysate with a compound of formula I

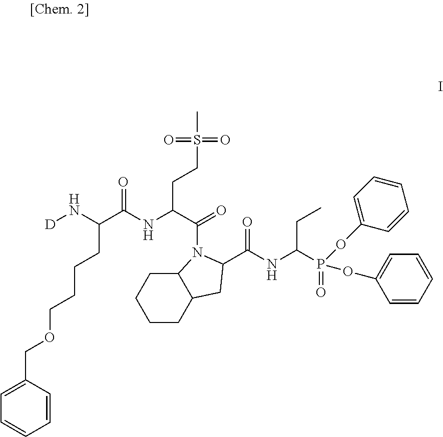

or a salt thereof,

wherein D is a detectable element,

(3) subsequently subjecting the lysate to gel electrophoresis; and thereafter

(4) measuring a detectable signal.

In certain embodiments, the present invention is directed to an in vitro method of inhibiting NE, comprising

(1) preparing a lysate from a tissue sample obtained from a subject,

(2) contacting the lysate with a compound of formula I

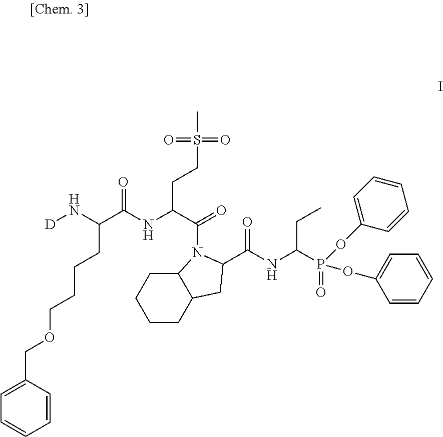

or a salt thereof,

wherein D is a detectable element.

In certain embodiments, the present invention is directed to the preceding methods wherein in step (2), the lysate is contacted with a compound having the formula IA:

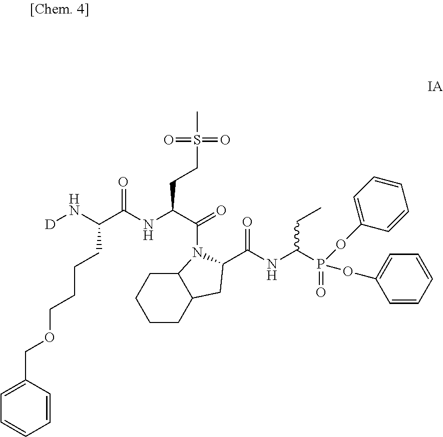

or a salt thereof,

wherein D is a detectable element.

In certain embodiments, the present invention is directed to an in vitro method of diagnosing an inflammatory bowel disease in a subject, comprising detecting an activated form of NE that is a trimmed form of mature NE.

In certain embodiments, the present invention is directed to a compound of formula I

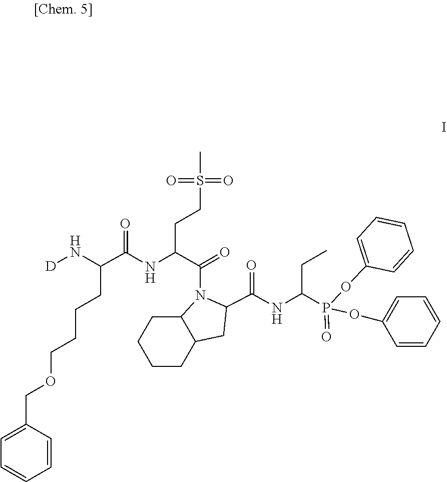

or a salt thereof,

wherein D is a detectable element,

with the proviso that compounds wherein D corresponds to one of the following formulas are excluded:

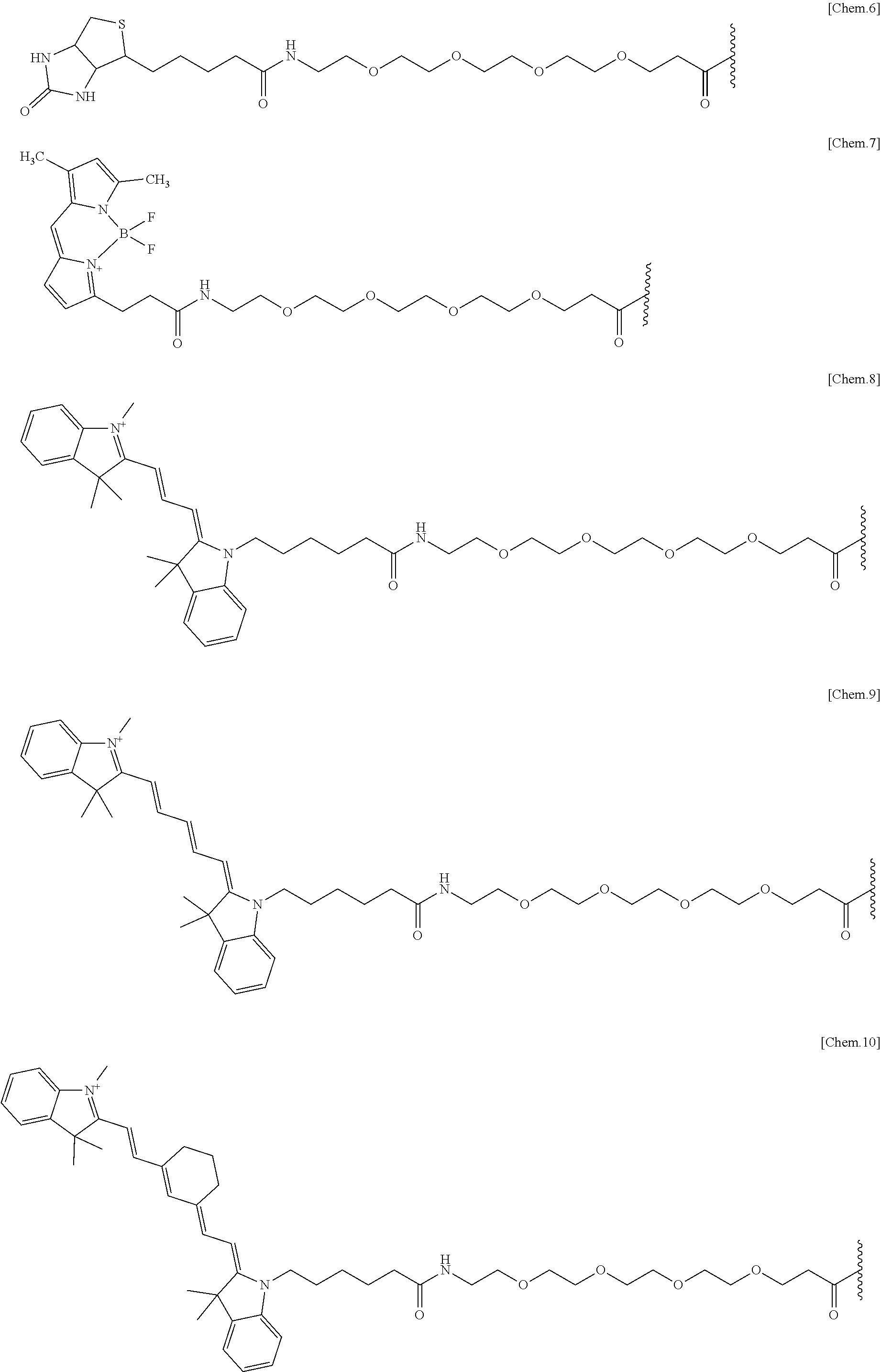

wherein in each of the above formulas, the curled line represents the point of connection to the remainder of the molecule. In certain such embodiments, the present invention is directed to a compound having the formula IA:

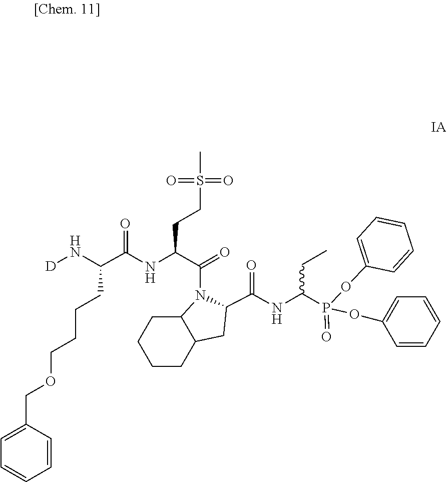

or a salt thereof, wherein D is a detectable element.

In certain embodiments, the present invention is directed to a compound of formula II

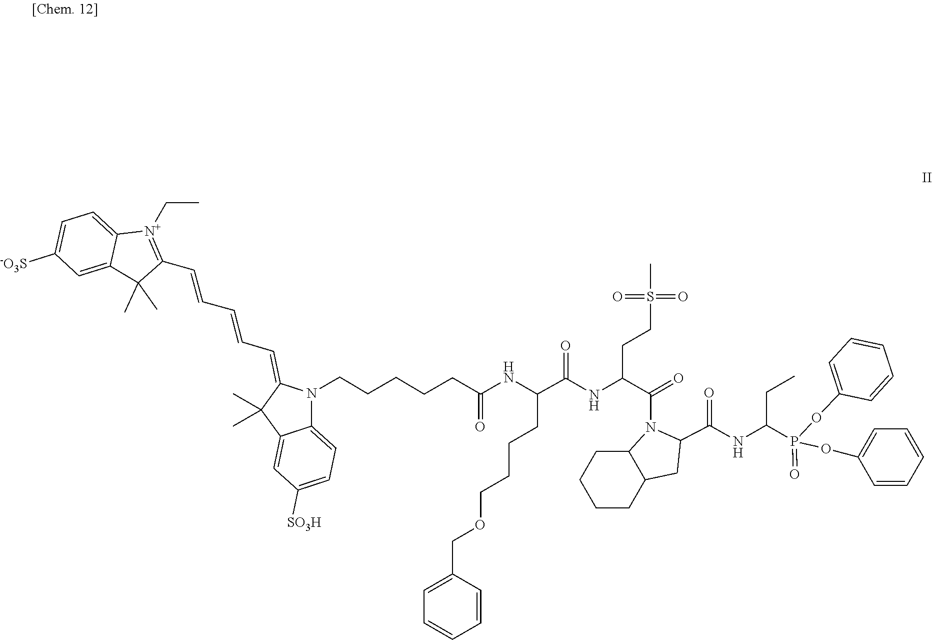

or a salt thereof.

In certain embodiments, the present invention is directed to a compound of formula IIA

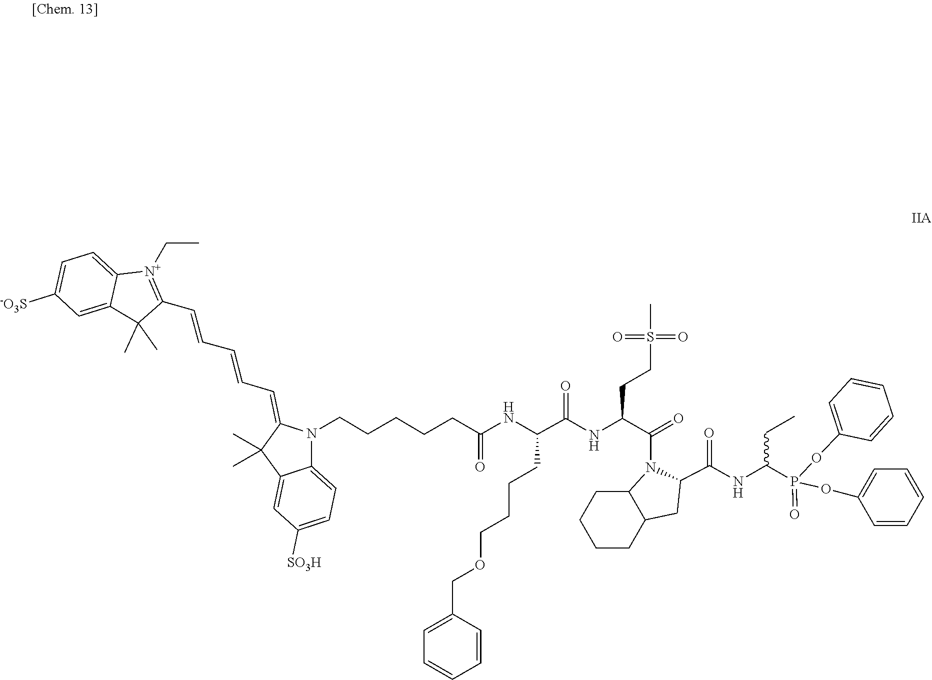

or a salt thereof.

In certain embodiments, the present invention is directed to a composition comprising any one of the above compounds or a salt thereof, and an excipient.

### BRIEF DESCRIPTION OF DRAWINGS

FIG. 1A depicts the purity of synthesized sulfoCy5-Nle(OBzl)-Met(O)₂-Oic-OH through measurement of absorbance at 214 nm by HPLC.

FIG. 1B depicts API-ES analysis of synthesized sulfoCy5-Nle(OBzl)-Met(O)₂-Oic-OH: m/z calculated; C₆₀H₇₉N₅O₁₄S₃ [M-H]⁻ 1189.5, [M-2H]²⁻ 593.7; observed: [M-H]⁻ 1189.0, [M-2H]²⁻ 593.6.

FIG. 2A depicts the purity of synthesized PK105b through measurement of absorbance at 214 nm by HPLC.

FIG. 2B depicts API-ES analysis of PK105b: m/z calculated; C₇₅H₉₅N₆O₁₆PS₃ [M-H]⁻ 1462.8, [M-2H]^{2 -} 730.3; observed: [M-H]⁻ 1462.8, [M-2H]²⁻ 730.2.

FIG. 3 depicts in-gel fluorescence results for concentration-dependent binding of Cy5-V-DPP and PK105b to recombinant serine proteases.

FIG. 4A depicts in-gel fluorescence results for murine bone marrow lysates labeled with Cy5-V-DPP or PK105b ex vivo.

FIG. 4B depicts results of immunoprecipitation of PK105b-labeled lysates from FIG. 4A with an NE-specific antibody.

FIG. 4C depicts in-gel fluorescence results for murine pancreas lysates labeled with Cy5-V-DPP or PK105b ex vivo.

FIG. 4D depicts results of immunoprecipitation of PK105b-labeled lysates from FIG. 4A with antibodies specific for NE, pancreatic elastase (PE), and trypsin 3 (Try3).

FIG. 5A depicts the results of ex vivo labeling of distal or proximal colons excised from mice with acute colitis induced by TNBS with PK105b detected by in-gel fluorescence (top; * indicates high-molecular weight species of unknown identity) and immunoblotting of the same samples with an NE-specific antibody to reveal total NE expression (bottom; n=3-5).

FIG. 5B depicts the results of immunoprecipitation of PK105b-labeled inflamed distal colon lysate with an NE-specific antibody.

FIG. 5C depicts results of in-gel fluorescence (top) and NE immunoblot (bottom) of distal colon lysates with or without PK105b labeling.

FIG. 6A depicts results of in-gel fluorescence of ex vivo labeled distal colon lysates (control or TNBS-treated) with Cy5-V-DPP probe.

FIG. 6B depicts results of in-gel fluorescence of luminal fluids from control or TNBS-treated mice labeled with PK105b.

FIG. 6C depicts results of in-gel fluorescence of fecal pellets from control or TNBS-treated mice labeled with PK105b.

FIG. 6D depicts the results of immunoprecipitation of PK105b-labeled fecal samples with antibodies specific for NE, PE, or trypsin 3.

FIG. 7A depicts the results of ex vivo labeling of mucosal biopsies from healthy patients or those with inflammatory bowel disease (IBD) with PK105b detected by in-gel fluorescence (top) and immunoblotting of the same samples with an NE-specific antibody to reveal total NE expression (bottom). Active ulcerative colitis (UC; n=9) or healthy controls (normal; n=5).

FIG. 7B depicts densitometry analysis of active NE (left) and the most trimmed species of NE detected by immunoblot (right; SEM).

FIG. 7C depicts the results of immunoprecipitation of PK105b-labeled UC biopsy lysates with an NE-specific antibody.

FIG. 8A depicts results of in-gel fluorescence of lungs excised from control or legionella-infected mice and labeled ex vivo with PK105b (* indicates high-molecular weight species of unknown identity) and immunoblotting the samples with an NE-specific antibody (n=3-5).

FIG. 8B depicts densitometry analysis of NE activity (left) and mature NE detected by immunoblot (right) in FIG. 8A.

FIG. 8C depicts the results of immunoprecipitation of PK105b-labeled lung lysates with an NE-specific antibody.

FIG. 9A depicts results of in-gel fluorescence of normal mouse tongues or HSC-3 oral squamous cell carcinoma xenografted tumors labeled ex vivo with PK105b (* indicates high-molecular weight species of unknown identity) and immunoblotting the samples with an NE-specific antibody (n=3-5).

FIG. 9B depicts densitometry analysis of NE activity (left) and mature NE detected by immunoblot (right) in FIG. 9A.

FIG. 9C depicts the results of immunoprecipitation of PK105b-labeled tumor lysates with an NE-specific antibody.

### DESCRIPTION OF EMBODIMENTS

In describing the present invention, the following terms are to be used as indicated below.

As used herein, the singular forms “a”, “an”, and “the” include plural references unless the context clearly indicates otherwise.

Unless defined otherwise, all technical and scientific terms used herein have the same meanings as commonly understood by one of ordinary skill in the art.

The term “neutrophil elastase activity” refers to proteolytic activity of the serine protease neutrophil elastase. Neutrophil elastase is also referred to as leukocyte elastase, elastase-2, serine elastase, subtype human leukocyte elastase (HLE), medullasin, PMN elastase, or bone marrow elastase.

The term “mature neutrophil elastase” or “mature NE” refers to a 25 kDa form resulting from trimming, i.e. shortening, of the 37 kDa inactive zymogen form that is initially produced from the NE gene, designated as ELANE or ELA2, by transcription and translation. Mature NE can exhibit NE activity.

The term “trimmed form of mature NE” refers to a form of NE that results from further trimming of mature NE and accordingly is <25 kDa.

The term “activated form of NE” refers to forms of NE that can exhibit NE activity. Mature NE and trimmed forms of mature NE can be activated forms of NE.

The term “tissue sample” or “tissue biopsy” refers to a sample of a biological tissue obtained from a subject, such as a sample obtained by excision, needle aspiration, biopsy forceps, or swab. Tissue samples also comprise mucosal biopsies, sputum samples, and fecal samples. The sampled tissue can be live, dead, healthy, or diseased and contain a heterogenous mixture of cell types and extracellular factors.

A “mucosal biopsy” is typically obtained by swabbing mucus accumulated on the surface of another tissue, e.g. mucous membranes or intestinal tract epithelia. Mucosal biopsies contain shed cells and cell excretions from the tissue the mucus accumulated on.

The term “sputum sample” refers to a sample that is a mixture of saliva and mucus coughed up from the respiratory tract. A “sputum sample” can be obtained invasively or non-invasively. Invasive methods involve oropharyngeal or endotracheal suctioning while the subject is intubated, and the obtained contents are collected in a sputum trap. Non-invasive methods collect the contents produced when the subject coughs, sometimes after nebulization with saline to loosen secretions.

The term “fecal sample” or “stool sample” refers to a sample collected from the feces of a subject. Fecal samples comprise cells shed from the gastrointestinal tract and cell excretions from the gastrointestinal tract of the subject.

The term “tissue sample lysate” refers to a solution obtained by lysing the cells of a tissue sample. The term “lysing” or “lysis” refers to the disintegration or rupture of the cell membranes, resulting in the release of cell contents and/or the subsequent death of the cell. Lysis can be accomplished e.g. by mechanical, enzymatic, or osmotic disruption of the cell membranes.

The term “activity-based probe” is intended to have the same meaning as commonly understood by one of ordinary skill in the art. Activity-based probes (ABPs) are small molecules that covalently bind to the active site of an enzyme (such as a protease) or a group of enzymes in an activity-dependent manner (i.e., the labeling reaction requires enzyme activity). ABPs typically include three elements: (i) an electrophilic moiety called “warhead”, (ii) a linker or recognition sequence, and (iii) a detectable element or “reporter moiety” for detection. The enzyme attacks the electrophilic warhead resulting in the formation of a covalent adduct which can then be detected either directly (e.g., if the detectable element is a fluorescent label), or by two-step labeling (e.g., post-labeling modification of a ligation handle).

The term “detectable element” or “reporter group/moiety” refers to a functional group in a compound (activity-based probe) that can be detected using techniques including, but not limited to, optical methods (e.g., measurement of fluorescence or UV-VIS absorbance), radiography, biochemical methods (e.g., using an immunochemical reagent such as an antibody), etc. The term “detectable element” includes functional groups that can be detected “directly” (e.g., by fluorescence measurement after running an SDS-PAGE) as well as functional groups that can be detected after performing a secondary labeling step and subsequent detection of the secondary label. An example for such groups is a biotin label which can be detected, e.g., after secondary labeling with fluorescently tagged streptavidin and subsequent fluorescence measurement. A further example for such groups is a click-chemistry label (bioorthogonal ligation handle) which can be detected, e.g., after secondary labeling with a fluorescent label using a click-chemistry (bioorthogonal) reaction and subsequent fluorescence measurement.

A “bioorthogonal ligation handle” is thus a functional group present in the compounds of the invention at the initial probe labeling step (in vivo or ex vivo contacting of the protease/biological sample/subject with the compounds of the invention), which enables the subsequent attachment of a secondary label (corresponding to the actually detected label) in a secondary labeling step using e.g. a click-chemistry (bioorthogonal) reaction which is performed in vitro.

Click-chemistry labels and respective click-chemistry reactions for secondary labeling, i.e., attachment of the label to be actually detected, are described, e.g., in Martell et al., Molecules (2014), and in Willems et al., Acc. Chem. Res. (2011).

Detectable elements give rise to “detectable signals” that can be measured in an analytical detection method as described herein.

The term “patient” means a subject, particularly a human subject, who has presented a clinical manifestation of a particular symptom or symptoms suggesting the need for treatment, who is treated preventatively or prophylactically for a condition, or who has been diagnosed with a condition to be treated.

The term “subject” is meant to comprise mammalian subjects, in particular human subjects, and is inclusive of the definition of the term “patient” and does not exclude individuals who are entirely normal in all respects or with respect to a particular condition.

The term “disease associated with neutrophil elastase activity” or “disease associated with NE activity” as used herein denotes a disease wherein neutrophil elastase activity is implicated in the pathogenesis of the disease. In a “disease associated with NE activity”, the level of NE activity in the diseased state or diseased region of the body (e.g., body part, organ, pathological tissue including tumor tissue), deviates from the respective level of NE activity found in the pathology-free state or in the respective pathology-free region of the body. In certain embodiments, the level of NE activity in the diseased state or diseased region of the body, is increased as compared to the respective level of NE activity found in the pathology-free state or in the respective pathology-free region of the body. For example, in the pathology-free state or region, the level of NE activity can be below a detectable limit, whereas in the diseased state or region, the level of NE activity is above the detectable limit. Diseases associated with neutrophil activity are, e.g., celiac disease, gastrointestinal motility disorders, pain, itch, skin disorders such as topic dermatitis, diet-induced obesity, metabolic disorders (including, but not limited to nonalcoholic steatohepatitis (NASH), hepatic and pancreatic disease), asthma, rheumatoid arthritis, periodontitis, inflammatory GI disorders (such as inflammatory bowel diseases (IBD), infectious diarrhea, mesenteric ischaemia, diverticulitis and necrotizing enterocolitis (NEC)), functional GI disorders (such as irritable bowel syndrome, functional chest pain, functional dyspepsia, nausea and vomiting disorders, functional constipation, functional diarrhea, fecal incontinence, functional anorectal pain, and functional defecation disorders), cancer, fibrotic diseases, metabolic dysfunction, neurological diseases, chronic obstructive pulmonary disease (COPD), and infection.

The term “inflammatory gastrointestinal disease”, “inflammatory gastrointestinal disorder”, or “inflammatory GI disease” as used herein denotes gastrointestinal diseases, i.e. diseases involving the gastrointestinal tract, namely the oral cavity, esophagus, stomach, small intestine, large intestine (colon) and rectum, and the accessory organs of digestion (e.g., the tongue, salivary glands, pancreas, liver and gallbladder), in which there is inflammation of one or more parts of the GI tract. Inflammatory GI diseases comprise, e.g., inflammatory bowel diseases, infectious diarrhea, mesenteric ischemia, diverticulitis, and necrotizing enterocolitis.

The term “inflammatory bowel disease” or “IBD” refers to a collection of diseases characterized by chronic and relapsing inflammation in the gastrointestinal tract. IBD most notably comprises ulcerative colitis (UC) and Crohn's disease (CD), both of which are associated with diarrhea, rectal bleeding, increased urgency, and pain, but also comprises less prevalent diseases such as acute colitis, immuno-oncology colitis, chemotherapy/radiation colitis, Graft versus Host Disease colitis, collagenous colitis, lymphocytic colitis, microscopic colitis, diversion colitis, Behcet's disease, and indeterminate colitis and pouchitis.

The term “functional gastrointestinal disorders”, “functional GI disorders” or “functional GI diseases” as used herein denotes disorders of gut-brain interaction. It is a group of disorders classified by GI symptoms related to any combination of the following: motility disturbance, visceral hypersensitivity, altered mucosal and immune function, altered gut microbiota, and altered central nervous system (CNS) processing. The term “functional” is generally applied to disorders in which the body's normal activities in terms of the movement of the intestines, the sensitivity of the nerves of the intestines, or the way in which the brain controls some of these functions is impaired. However, there are no structural abnormalities that can be seen by endoscopy, x-ray, or blood tests. Thus, these disorders are largely identified by the characteristics of the symptoms. Functional GI disorders comprise irritable bowel syndrome, functional chest pain, functional dyspepsia, nausea and vomiting disorders, functional constipation, functional diarrhea, fecal incontinence, functional anorectal pain, and functional defecation disorders.

The term “infection” refers to a process or state wherein an infectious agent (such as, e.g., pathogenic bacteria, fungi, protozoa, viruses, prions, viroids, nematodes, and helminths) invade and multiply in the body tissues of an infected subject.

The term “chronic obstructive pulmonary disease” refers to a group of progressive lung diseases and includes emphysema, chronic bronchitis, and refractory (non-reversible) asthma. These diseases are characterized by increasing breathlessness and poor air-flow.

The term “cancer” refers to a collection of diseases characterized by uncontrolled, abnormal growth of cells with the potential to invade or spread to other parts of the body. Cancer can affect any tissue and is named after the tissue of origin. The term “oral cancer” refers to cancers of the mouth, i.e. any cancerous tissue growth located in the oral cavity of a subject. Exemplary histological types of oral cancer are teratoma, adenocarcinoma derived from a major or minor salivary gland, lymphoma from tonsillar or other lymphoid tissue, or melanoma from the pigment-producing cells of the oral mucosa. The most common type of oral cancer is squamous cell carcinoma originating in the tissues that line the mouth and lips, with less common types including Kaposi's sarcoma. Oral cancer most commonly involves the tongue, but may also occur on the floor of the mouth, cheek lining, gingiva, lips, or palate. The term “breast cancer” refers to cancers of the breast. Exemplary breast cancers are ductal carcinoma in situ (DCIS), lobular carcinoma in situ (LCIS), invasive ductal carcinoma (IDC), invasive lobular carcinoma (ILC), Paget disease of the nipple, phyllodes tumor, and angiosarcoma. The term “prostate cancer” refers to cancer of the prostate. Exemplary prostate cancers include adenocarcinomas of the prostate. The term “colorectal cancer” refers to cancers of the colon and/or rectum. Exemplary colorectal cancers are adenocarcinomas, carcinoid tumors, gastrointestinal stromal tumors (GISTs), lymphomas, and sarcomas originating from the colon or rectum.

The term “(C_{y}-C_{z})” when used in conjunction with a chemical group, such as alkyl and aryl, indicates the possible number of carbon atoms in the group (i.e., from y to z carbon atoms).

The term “alkyl” as used herein denotes a straight-chain or branched alkyl group. Examples of alkyl groups include methyl, ethyl, n-propyl, iso-propyl, n-butyl, 2-butyl, iso-butyl, tert-butyl, n-pentyl, 1-methylbutyl, 2-methylbutyl, 3-methylbutyl, and 2,2-dimethylpropyl, etc. In certain embodiments the term “alkyl” denotes a straight-chain alkyl group, such as methyl, ethyl, n-propyl, n-butyl, n-pentyl, n-hexyl, n-heptyl and n-octyl.

The term “aryl” as used herein denotes groups derived from monocyclic or polycyclic aromatic hydrocarbons by removal of a hydrogen atom from a ring carbon atom. Examples of aryl groups include phenyl and naphtyl.

The term “sulfo” as used herein is art recognized and refers to the group —SO₃H, or a salt form thereof.

Formulas indicating positively or negatively charged atoms or groups (such as N⁺ or SO₃⁻) mean salt forms of the respective formula (including “inner salts” in the case of zwitterions).

For purposes of the present invention, the term “salt” includes inorganic acid salts, such as hydrochloride, hydrobromide, sulfate, phosphate and the like; and organic acid salts, such as myristate, formate, acetate, trifluoroacetate, maleate, tartrate, bitartrate and the like; sulfonates, such as, methanesulfonate, benzenesulfonate, p-toluenesulfonate and the like; and amino acid salts such as arginate, asparaginate, glutamate and the like. The term “salt” includes solvates, such as hydrates, of the respective salt.

In certain embodiments, the term “salt” as used herein means a diagnostically acceptable salt. In certain embodiments, the term “salt” as used herein means a diagnostically and pharmaceutically acceptable salt.

The term “pharmaceutically acceptable salt”, as used herein, means a salt of a compound of the present invention which is safe and effective for topical or systemic use in mammals and that possesses the desired biological activity. The counter ion is suitable for the intended use, non-toxic, and it does not interfere with the desired biological action of the compound. Pharmaceutically acceptable salts in the context of the present invention include the salts reviewed in the IUPAC Handbook of Pharmaceutically Acceptable Salts (Wermuth, C. G. and Stahl, P. H., Pharmaceutical Salts: Properties, Selection and Use—A Handbook, Verlag Helvetica Chimica Acta (2002)).

The term “diagnostically acceptable salt”, as used herein, refers to a salt of a compound of the present invention which is useful and effective for the desired diagnostic method. Its counter ion does not interfere with the reaction necessary for detection of the target protein, or with the method of detection/diagnosis.

In certain embodiments the compounds of the present invention are present as the trifluoroacetate salt, e.g., after HPLC-purification in an eluting solvent including trifluoroacetic acid (TFA).

As used herein, the term “contacting the lysate with a compound of formula I (or a salt thereof)” also encompasses embodiments wherein the lysate is contacted with a composition comprising the compound of formula I (or a salt thereof) and an excipient. In certain such embodiments the composition is an aqueous solution comprising e.g. water, physiologically buffered saline or a buffer solution as excipient. In certain such embodiments the aqueous solution additionally comprises detergents such as triton X-100.

In certain embodiments, “excipient” means a diagnostically and/or pharmaceutically acceptable excipient. Diagnostically and/or pharmaceutically acceptable excipients that can be used in the compositions of the present invention are known to the skilled person. Examples of such pharmaceutically acceptable excipients include, e.g. those described in paragraphs [0114] to [0118] of WO 2018/119476, the contents of which are hereby introduced into the present disclosure.

In formulas showing a curled line neighboring a chemical structure, the curled line represents or indicates the point of connection to the remainder of the molecule. Where a bond within a chemical structure is drawn as a curled line (as in formula IA and IIA as described herein), this indicates that the stereochemistry at the respective position is not defined, i.e., the substituent attached by this bond (in formula IA and IIA, the ethyl group) can point to the back or to the front.

A compound of formula I or II can contain one or more asymmetric centers and can thus give rise to enantiomers, diastereomers, and other stereoisomeric forms. Unless specifically otherwise indicated, the disclosure encompasses compounds with all such possible forms, as well as their racemic and resolved forms or any mixture thereof. When a Compound of formula (I) contains an olefinic double bond or other center of geometric asymmetry, and unless specifically otherwise indicated, it is intended to include all “geometric isomers”, e.g., both E and Z geometric isomers. Unless specifically otherwise indicated, all “tautomers”, e.g., ketone-enol, amide-imidic acid, lactam-lactim, enamine-imine, amine-imine, and enamine-enimine tautomers, are intended to be encompassed by the disclosure as well.

As used herein, the terms “stereoisomer”, “stereoisomeric form”, and the like are general terms for all isomers of individual molecules that differ only in the orientation of their atoms in space. It includes enantiomers and isomers of compounds with more than one chiral center that are not minor images of one another (“diastereomers”).

The term “chiral center” refers to a carbon atom to which four different groups are attached.

The term “enantiomer” or “enantiomeric” refers to a molecule that is nonsuperimposeable on its mirror image and hence optically active where the enantiomer rotates the plane of polarized light in one direction and its minor image rotates the plane of polarized light in the opposite direction.

The term “racemic” refers to a mixture of equal parts of enantiomers which is optically inactive.

The term “resolution” refers to the separation or concentration or depletion of one of the two enantiomeric forms of a molecule. Optical isomers of a Compound of Formula (I) can be obtained by known techniques such as chiral chromatography or formation of diastereomeric salts from an optically active acid or base.

Optical purity can be stated in terms of enantiomeric excess (% ee), which is determined by the formula:

$$\% ee [ \frac{major enantiomer ( mol ) - minor enantiomer ( mol )}{major enantiomer ( mol ) + minor enantiomer ( mol )} ] Χ 100 \% \qquad [ Math .1 ]$$

In one embodiment the invention relates to compounds having the absolute stereochemistry indicated by formulas IA or IIA.

The compounds of the present invention can be synthesized using standard synthetic chemical techniques, for example using the methods described in the Examples section below. Other useful synthetic techniques are described, for example, in March's Advanced Organic Chemistry: Reactions, Mechanisms, and Structure, 7th Ed., (Wiley, 2013); Carey and Sundberg, Advanced Organic Chemistry 4th Ed., Vols. A and B (Plenum 2000, 2001); Fiesers' Reagents for Organic Synthesis, Volumes 1-27 (Wiley, 2013); Rodd's Chemistry of Carbon Compounds, Volumes 1-5 and Supplementals (Elsevier Science Publishers, 1989); Organic Reactions, Volumes 1-81 (Wiley, 2013); and Larock's Comprehensive Organic Transformations (VCH Publishers Inc., 1989) (all of which are incorporated by reference in their entirety). The compounds are normally synthesized using starting materials that are generally available from commercial sources or are readily prepared using methods well known to those skilled in the art. See, e.g., Fiesers' Reagents for Organic Synthesis, Volumes 1-27 (Wiley, 2013), or Beilsteins Handbuch der organischen Chemie, 4, Aufl. ed. Springer-Verlag, Berlin, including supplements.

Methods of Detecting Neutrophil Elastase Activity

In the methods of detecting neutrophil elastase activity according to the present invention, only proteolytically active forms of neutrophil elastase are detected.

The detectable signal is measured after a reaction between the activity-based probe compound and neutrophil elastase has taken place, which has resulted in the formation of a covalent bond. The measured detectable signal is emitted by the labeled enzyme, i.e. by the detectable element of the activity-based probe compound covalently attached to the neutrophil elastase. In certain embodiments the detectable signal is measured after subjecting the labeled enzyme to a secondary labeling step.

The concept of detecting enzyme activity using activity-based probes and respective methods of detection and underlying experimental protocols are known to the skilled person (see, e.g., Edgington and Bogyo, 2013; Edgington-Mitchell, L. E., and Bogyo, M. (2016). Detection of Active Caspases During Apoptosis Using Fluorescent Activity-Based Probes. Methods Mol Biol. 1419, 27-39; and Edgington-Mitchell, L. E., Bogyo, M., and Verdoes, M. (2017). Live Cell Imaging and Profiling of Cysteine Cathepsin Activity Using a Quenched Activity-Based Probe. Methods Mol Biol. 1491, 145-159; the contents of which are hereby incorporated by reference in their entirety). The skilled person knows how to suitably adapt these methods/protocols for use in the methods of the present invention.

In certain embodiments, the present invention is directed to a method of detecting neutrophil elastase (NE) activity in a tissue sample lysate, comprising

(1) preparing the lysate from a tissue sample obtained from a subject,

(2) contacting the lysate with a compound of formula I

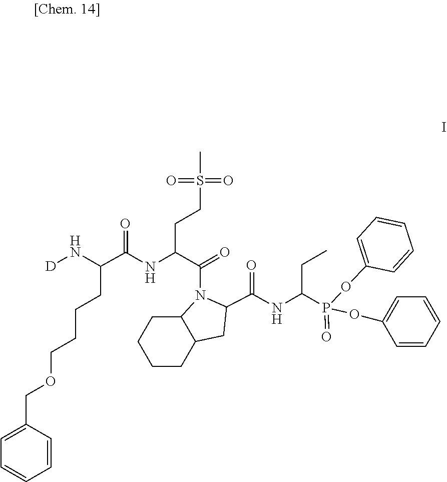

or a salt thereof,

wherein D is a detectable element,

(3) subsequently subjecting at least an aliquot of the lysate of step (2) to gel electrophoresis; and thereafter

(4) measuring a detectable signal.

In certain such embodiments, the present invention is directed to a method further comprising after step (3) a step

(5) immunoblotting with an anti-NE antibody.

In certain embodiments, the present invention is directed to a method, wherein additionally the following steps are performed:

(3a) immunoprecipitating the compound of formula I in a separate aliquot of the lysate of step (2) using an antibody specific for the compound of formula I or a part thereof (i.e. specific for a part of the compound of formula I),

(4a) subsequently analyzing co-precipitated material.

In certain such embodiments, the present invention is directed to a method, wherein the analysis of step (4a) comprises
gel electrophoresis and subsequent immunoblot using an anti-NE antibody, or
protein sequencing,
   and preferably comprises gel electrophoresis and subsequent immunoblot using an anti-NE antibody.

In certain embodiments, the present invention is directed to a method of any one of the preceding embodiments, wherein prior to step (2), an aliquot of the lysate of step (1) is pretreated with a specific NE inhibitor, and wherein the pretreated aliquot is subsequently processed analogously to the not pretreated lysate of step (1).

In certain embodiments, the present invention is directed to a method of any one of the preceding embodiments, wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy. In certain such embodiments, the tissue sample is a mucosal biopsy and the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

In certain embodiments, the present invention is directed to a method of any one of the preceding embodiments, wherein the subject is a human subject.

In certain embodiments, the present invention is directed to a method of any one of the preceding embodiments, wherein an activated form of NE that is a trimmed form of mature NE is detected. In certain such embodiments, the present invention is directed to a method, wherein the tissue sample is selected from the group consisting of an oral biopsy, an esophagus sample, a stomach sample, a small intestine sample, a colon sample, a proximal colon sample, a distal colon sample, a rectal sample, a fecal sample and a mucosal biopsy. In certain such embodiments, the tissue sample is a mucosal biopsy, and the mucosal biopsy is selected from the group consisting of an oral mucosal biopsy, an esophagus mucosal biopsy, a small intestine mucosal biopsy, a colon mucosal biopsy, and a rectal mucosal biopsy.

In certain embodiments of the above methods, preparing the lysate comprises a clearing step. The clearing step may comprise a step of sedimentation of undissolved matter by gravity or centrifugation.

In certain embodiments of the above methods, the gel electrophoresis is a one-dimensional or a two-dimensional gel electrophoresis. In certain such embodiments, the gel electrophoresis is an SDS-PAGE or a native PAGE, preferably an SDS-PAGE.

In certain embodiments of the above methods, the detectable element is selected from the group consisting of a fluorescent label, a biotin label, a radiolabel, a chelator, and a bioorthogonal ligation handle. In certain such embodiments, the detectable signal is measured by fluorescence measurement or radiography. In certain such embodiments, the measurement is by fluorescence measurement, and the fluorescence measurement is in-gel fluorescence. In certain such embodiments, the fluorescence measurement is preceded by secondary labeling. In certain such embodiments, the secondary labeling is selected from the group consisting of secondary labeling with tagged streptavidin, secondary labeling with a fluorophore, and secondary labeling with a tagged antibody.

In certain embodiments of each of the above methods, the measurement of detectable signal in step (4) comprises a measurement selected from the group consisting of radiography, and gel electrophoresis and subsequent radiography. In certain such embodiments, said compound comprises a detectable element in the form of a radiolabel. In certain other embodiments, said compound comprises a detectable element in the form of a chelator for a radiolabel. In certain other embodiments, said compound comprises a detectable element in the form of a bioorthogonal ligation handle, and step (4) further comprises secondary labeling by click-chemistry to apply a radiolabel or a chelator for a radiolabel prior to performing the radiography measurement.

In certain embodiments of each of the above methods, the measurement of detectable signal in step (4) comprises a measurement selected from the group consisting of affinity purification and subsequent mass spectrometry, and affinity purification and subsequent proteomics. In certain such embodiments, said compound comprises a detectable element in the form of a biotin label. In certain other embodiments, said compound comprises a detectable element in the form of a bioorthogonal ligation handle, and step (4) further comprises secondary labeling by click-chemistry to apply a biotin label prior to performing the affinity purification. In the case of biotin-labeling, affinity purification can be performed using, e.g., streptavidin-coated beads, or beads coated with an antibody specific for biotin.

In certain embodiments, the affinity purification can be performed using beads coated with an antibody specific for a certain tag. In certain such embodiments, said compound comprises said tag as a detectable element. In certain other embodiments, said compound comprises a detectable element in the form of a bioorthogonal ligation handle, and step (4) further comprises secondary labeling by click-chemistry to apply said tag prior to performing the affinity purification.

In certain embodiments of each of the above methods, the measurement of detectable signal in step (4) comprises gel electrophoresis and subsequent immunoblotting. In certain such embodiments, said compound comprises a detectable element in the form of a biotin label, and step (4) further comprises secondary labeling, e.g., with HRP-tagged-streptavidin prior to performing the immunoblot. In certain other embodiments, said compound comprises a detectable element in the form of a bioorthogonal ligation handle, and step (4) further comprises secondary labeling by click-chemistry to apply a biotin label and subsequent labeling, e.g., with HRP-tagged-streptavidin prior to performing the immunoblot.

Methods of Diagnosis

In certain embodiments, the invention is directed to an in vitro method of diagnosing an inflammatory bowel disease (IBD) in a subject, comprising detecting an activated form of NE that is a trimmed form of mature NE. In certain such embodiments, the subject is a human subject. In certain embodiments, the invention is directed to an in vitro method of diagnosing an inflammatory bowel disease in a subject, wherein the method comprises a step of contacting the activated form of NE with an activity-based probe. In certain embodiments, the invention is directed to an in vitro method of diagnosing an inflammatory bowel disease in a subject, wherein the method comprises a step of contacting the activated form of NE with an anti-NE-antibody.

In certain other embodiments, the present invention is directed to an in vitro method of diagnosing a disease associated with NE activity in a subject, comprising

(1) preparing a lysate from a tissue sample obtained from the subject,

(2) contacting the lysate with a compound of formula I

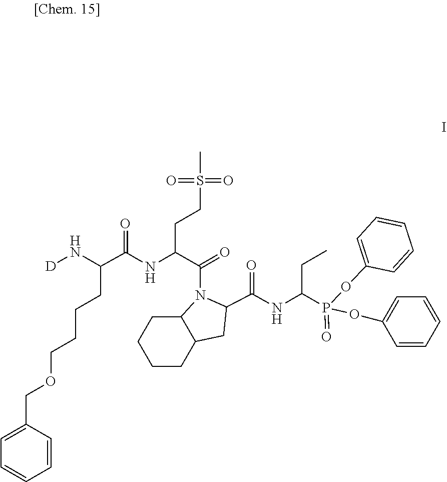

or a salt thereof,

wherein D is a detectable element,

(3) subsequently subjecting the lysate to gel electrophoresis; and thereafter

(4) measuring a detectable signal. In certain such embodiments, the disease is selected from the group consisting of an infection (such as a wound infection or a lung infection), an inflammatory disease (such as an inflammatory bowel disease), an autoimmune disease (such as diabetes), a chronic obstructive pulmonary disease, and a cancer. In certain such embodiments, the above method further comprises after step (3) a step

(5) immunoblotting with an anti-NE antibody.

In certain such embodiments, the disease associated with NE activity is selected from the group consisting of a celiac disease, a gastrointestinal motility disorder, pain, itch, a skin disorder, diet-induced obesity, a metabolic disorder, asthma, rheumatoid arthritis, periodontitis, an inflammatory GI disorder, a functional GI disorder, a cancer, a fibrotic disease, metabolic dysfunction, a neurological disease, a chronic obstructive pulmonary disease (COPD), and an infection.

In certain other such embodiments the disease associated with NE activity is selected from the group consisting of an inflammatory bowel disease, an infection, a chronic obstructive pulmonary disease, and a cancer.

In certain embodiments, the above method further comprises after step (3) a step (5) immunoblotting with an anti-NE antibody.

In certain embodiments, the invention is directed to a method wherein additionally the following steps are performed:

(3a) immunoprecipitating the compound of formula I in a separate aliquot of the lysate of step (2) using an antibody specific for the compound of formula I or a part thereof,

(4a) subsequently analyzing co-precipitated material. In certain such embodiments, the analysis of step (4a) comprises
gel electrophoresis and subsequent immunoblot using an anti-NE antibody, or
protein sequencing,

and preferably comprises gel electrophoresis and subsequent immunoblot using an anti-NE antibody.

In certain embodiments, the invention is directed to a method of diagnosis of any one of the preceding embodiments, wherein prior to step (2), an aliquot of the lysate of step (1) is pretreated with a specific NE inhibitor, and wherein the pretreated aliquot is subsequently processed analogously to the not pretreated lysate of step (1).

In certain embodiments of the preceding methods of diagnosis, the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy. In certain such embodiments, the tissue sample is a mucosal biopsy, and the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

In certain embodiments, the invention is directed to a method of diagnosis of any one of the preceding embodiments, wherein the subject is a human subject.

Inflammatory Bowel Disease

In certain embodiments, the invention is directed to a method of diagnosis of any one of the preceding embodiments, wherein the method is for diagnosing an inflammatory bowel disease. In certain such embodiments, an activated form of NE that is a trimmed form of mature NE is detected. In certain such embodiments, the subject is diagnosed as having inflammatory bowel disease if the activated form of NE is detected.

In certain embodiments, the invention is directed to a method of diagnosis of inflammatory bowel disease of any one of the above embodiments, wherein the tissue sample is selected from the group consisting of an oral biopsy, an esophagus sample, a stomach sample, a small intestine sample, a colon sample, a proximal colon sample, a distal colon sample, a rectal sample, a fecal sample, and a mucosal biopsy. In certain such embodiments, the tissue sample is a mucosal biopsy, and the mucosal biopsy is selected from the group consisting of an oral mucosal biopsy, an esophagus mucosal biopsy, a small intestine mucosal biopsy, a colon mucosal biopsy, and a rectal mucosal biopsy.

In certain embodiments, the invention is directed to a method of diagnosis of inflammatory bowel disease of any one of the above embodiments, wherein the inflammatory bowel disease is selected from the group consisting of acute colitis, ulcerative colitis, Crohn's disease, microscopic colitis, diversion colitis, Behcet's disease, immuno-oncology colitis, chemotherapy/radiation colitis, Graft versus Host Disease colitis, collagenous colitis, lymphocytic colitis, and indeterminate colitis and pouchitis.

In certain embodiments, the invention is directed to a method of diagnosis of inflammatory bowel disease of any one of the above embodiments, wherein the inflammatory bowel disease is ulcerative colitis. In certain such embodiments, the tissue sample is a colon sample, a proximal colon sample, a distal colon sample, or a colon mucosal biopsy.

In other certain embodiments, the invention is directed to a method of diagnosis of inflammatory bowel disease of any one of the above embodiments, wherein the inflammatory bowel disease is Crohn's disease. In certain such embodiments, the tissue sample is selected from the group consisting of an oral biopsy, an esophagus sample, a stomach sample, a small intestine sample, a colon sample, a proximal colon sample, a distal colon sample, a rectal sample, a fecal sample and a mucosal biopsy. In certain such embodiments, the tissue sample is a mucosal biopsy, and the mucosal biopsy is selected from the group consisting of an oral mucosal biopsy, an esophagus mucosal biopsy, a small intestine mucosal biopsy, a colon mucosal biopsy, and a rectal mucosal biopsy.

Infection

In other certain embodiments, the invention is directed to a method of diagnosis, wherein the method is for diagnosing an infection. In certain such embodiments, the infection is selected from the group consisting of a bacterial infection and a fungal infection. In certain such embodiments, the tissue sample is a sample from an infected tissue. In certain embodiments, the infected tissue is selected from the group consisting of a wound sample (e.g. wound fluid), a lung sample, a lung mucosal biopsy, and a sputum sample.

In certain embodiments, the invention is directed to a method of diagnosis of infection of any one of the above embodiments, wherein the infection is an infection of the lung. In certain such embodiments, the infection of the lung is a bacterial infection. In certain such embodiments, the bacterial infection is an infection with Legionella.

In certain embodiments, the invention is directed to a method of diagnosis of a lung infection of any one of the above embodiments, wherein the tissue sample is selected from the group consisting of a lung sample, a lung mucosal biopsy, and a sputum sample.

Cancer

In yet other certain embodiments the invention is directed to a method of diagnosis wherein the method is for diagnosing a cancer. In certain such embodiments, the tissue sample is selected from the group consisting of a tumor sample, an oral biopsy, an oral mucosal biopsy, a breast biopsy, a prostate biopsy, a colon biopsy, a colon mucosal biopsy, a rectal biopsy, a rectal mucosal biopsy, a lung biopsy, a lung mucosal biopsy and a sputum sample.

In certain embodiments, the invention is directed to a method of diagnosis of a cancer of any one of the above embodiments, wherein the cancer is selected from the group consisting of an oral cancer, a breast cancer, a prostate cancer, a colorectal cancer, and a lung cancer. In certain such embodiments, the cancer is an oral cancer. In certain such embodiments, the oral cancer is a squamous cell carcinoma.

In certain embodiments relating to the diagnosis of a cancer, wherein the cancer is breast cancer, the tissue is a sample as described above which is obtained from the breast of a subject, e.g. from a breast tumor.

In certain embodiments relating to the diagnosis of a cancer, wherein the cancer is lung cancer, the tissue is a sample, a sputum sample, or mucosal biopsy as described above which is obtained from the lung of a subject, e.g. from a lung tumor.

In certain embodiments relating to the diagnosis of a cancer, wherein the cancer is prostate cancer, the tissue is a sample as described above which is obtained from the prostate of a subject, e.g. from a prostate tumor.

In certain embodiments relating to the diagnosis of a cancer, wherein the cancer is oral cancer, the tissue is a sample or mucosal biopsy as described above which is obtained from the oral cavity of a subject, e.g. from an oral tumor.

In certain embodiments relating to the diagnosis of a cancer, wherein the cancer is colorectal cancer, the tissue is a sample or mucosal biopsy as described above which is obtained from the colon or rectum of a subject, e.g. from a colon or rectal tumor.

Chronic Obstructive Pulmonary Disease

In yet other certain embodiments the invention is directed to a method of diagnosis, wherein the method is for diagnosing a chronic obstructive pulmonary disease. In certain such embodiments, the tissue sample is selected from the group consisting of a lung sample, a lung mucosal biopsy, and a sputum sample.

In certain embodiments of the above methods, preparing the lysate comprises a clearing step. The clearing step may comprise a step of sedimentation of undissolved matter by gravity or centrifugation.

In certain embodiments of each of the above methods of diagnosis, the gel electrophoresis is a one-dimensional or a two-dimensional gel electrophoresis. In certain such embodiments, the gel electrophoresis is an SDS-PAGE or a native PAGE, preferably an SDS-PAGE.

In certain embodiments of the above methods, the detectable element is selected from the group consisting of a fluorescent label, a biotin label, a radiolabel, a chelator, and a bioorthogonal ligation handle. In certain such embodiments, the detectable signal is measured by fluorescence measurement or radiography. In certain such embodiments, the measurement is by fluorescence measurement, and the fluorescence measurement is in-gel fluorescence. In certain such embodiments, the fluorescence measurement is preceded by secondary labeling. In certain such embodiments, the secondary labeling is selected from the group consisting of secondary labeling with tagged streptavidin, secondary labeling with a fluorophore, and secondary labeling with a tagged antibody.

In certain embodiments of each of the above methods, the measurement of detectable signal in step (4) comprises a measurement selected from the group consisting of radiography, and gel electrophoresis and subsequent radiography. In certain such embodiments, said compound comprises a detectable element in the form of a radiolabel. In certain other embodiments, said compound comprises a detectable element in the form of a chelator for a radiolabel. In certain other embodiments, said compound comprises a detectable element in the form of a bioorthogonal ligation handle, and step (4) further comprises secondary labeling by click-chemistry to apply a radiolabel or a chelator for a radiolabel prior to performing the radiography measurement.

In certain embodiments of each of the above methods, the measurement of detectable signal in step (4) comprises a measurement selected from the group consisting of affinity purification and subsequent mass spectrometry, and affinity purification and subsequent proteomics. In certain such embodiments, said compound comprises a detectable element in the form of a biotin label. In certain other embodiments, said compound comprises a detectable element in the form of a bioorthogonal ligation handle, and step (4) further comprises secondary labeling by click-chemistry to apply a biotin label prior to performing the affinity purification. In the case of biotin-labeling, affinity purification can be performed using, e.g., streptavidin-coated beads, or beads coated with an antibody specific for biotin.

In certain embodiments, the affinity purification can be performed using beads coated with an antibody specific for a certain tag. In certain such embodiments, said compound comprises said tag as a detectable element. In certain other embodiments, said compound comprises a detectable element in the form of a bioorthogonal ligation handle, and step (4) further comprises secondary labeling by click-chemistry to apply said tag prior to performing the affinity purification.

In certain embodiments of each of the above methods, the measurement of detectable signal in step (4) comprises gel electrophoresis and subsequent immunoblotting. In certain such embodiments, said compound comprises a detectable element in the form of a biotin label, and step (4) further comprises secondary labeling, e.g., with HRP-tagged-streptavidin prior to performing the immunoblot. In certain other embodiments, said compound comprises a detectable element in the form of a bioorthogonal ligation handle, and step (4) further comprises secondary labeling by click-chemistry to apply a biotin label and subsequent labeling, e.g., with HRP-tagged-streptavidin prior to performing the immunoblot.

Methods of Inhibiting Neutrophil Elastase Activity

In certain embodiments, the present invention is directed to an in vitro method of inhibiting NE, comprising

(1) preparing a lysate from a tissue sample obtained from a subject,

(2) contacting the lysate with a compound of formula I

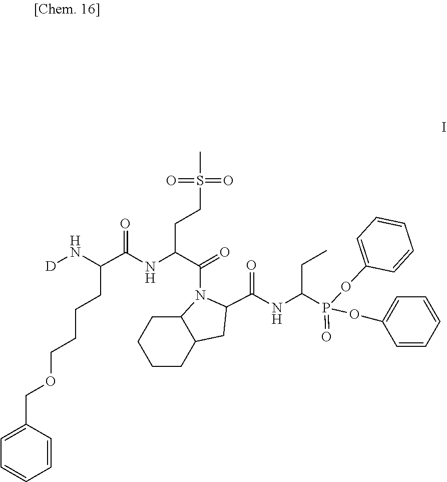

or a salt thereof,

wherein D is a detectable element.

In certain such embodiments, the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy. In certain such embodiments, the tissue sample is a mucosal biopsy, and the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

In certain embodiments, the invention is directed to an in vitro method of inhibiting neutrophil elastase activity of any one of the above embodiments, wherein the subject is a human subject.

In certain embodiments of the above methods, preparing the lysate comprises a clearing step. The clearing step may comprise a step of sedimentation of undissolved matter by gravity or centrifugation.

Compounds

The above described methods of detecting NE activity, methods of diagnosis, and in vitro methods of inhibiting NE, each comprise a step (2) of contacting the lysate with a compound of formula (I)

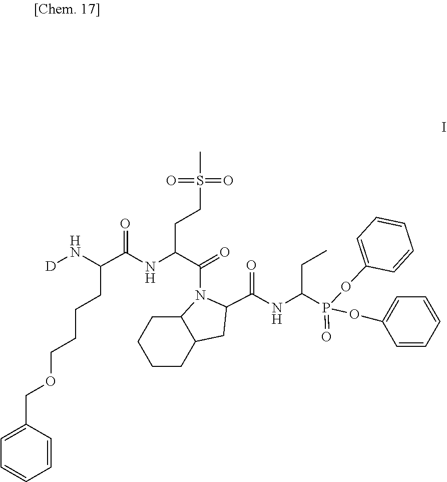

or a salt thereof, wherein D is a detectable element.

In certain such embodiments, in step (2), the lysate is contacted with a compound having the formula IA:

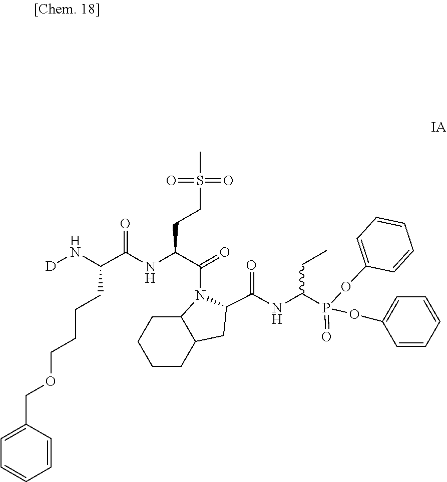

or a salt thereof, wherein D is a detectable element.

In certain embodiments the present invention also relates to a compound of formula I

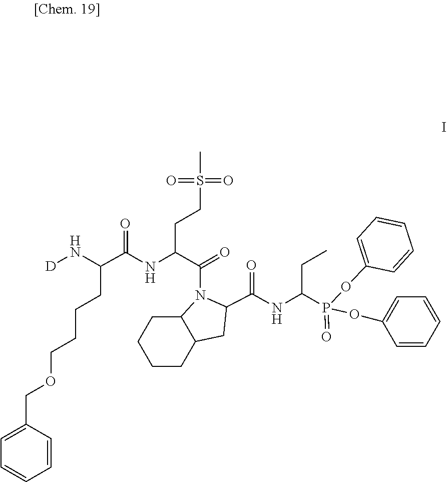

or a salt thereof,

or to a compound of formula IA:

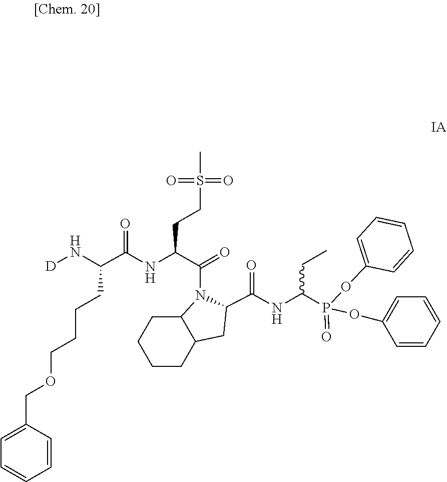

or a thereof,

wherein D is a detectable element,

with the proviso that compounds (of formula I or formula IA) wherein D corresponds to one of the following formulas are not encompassed by the present invention:

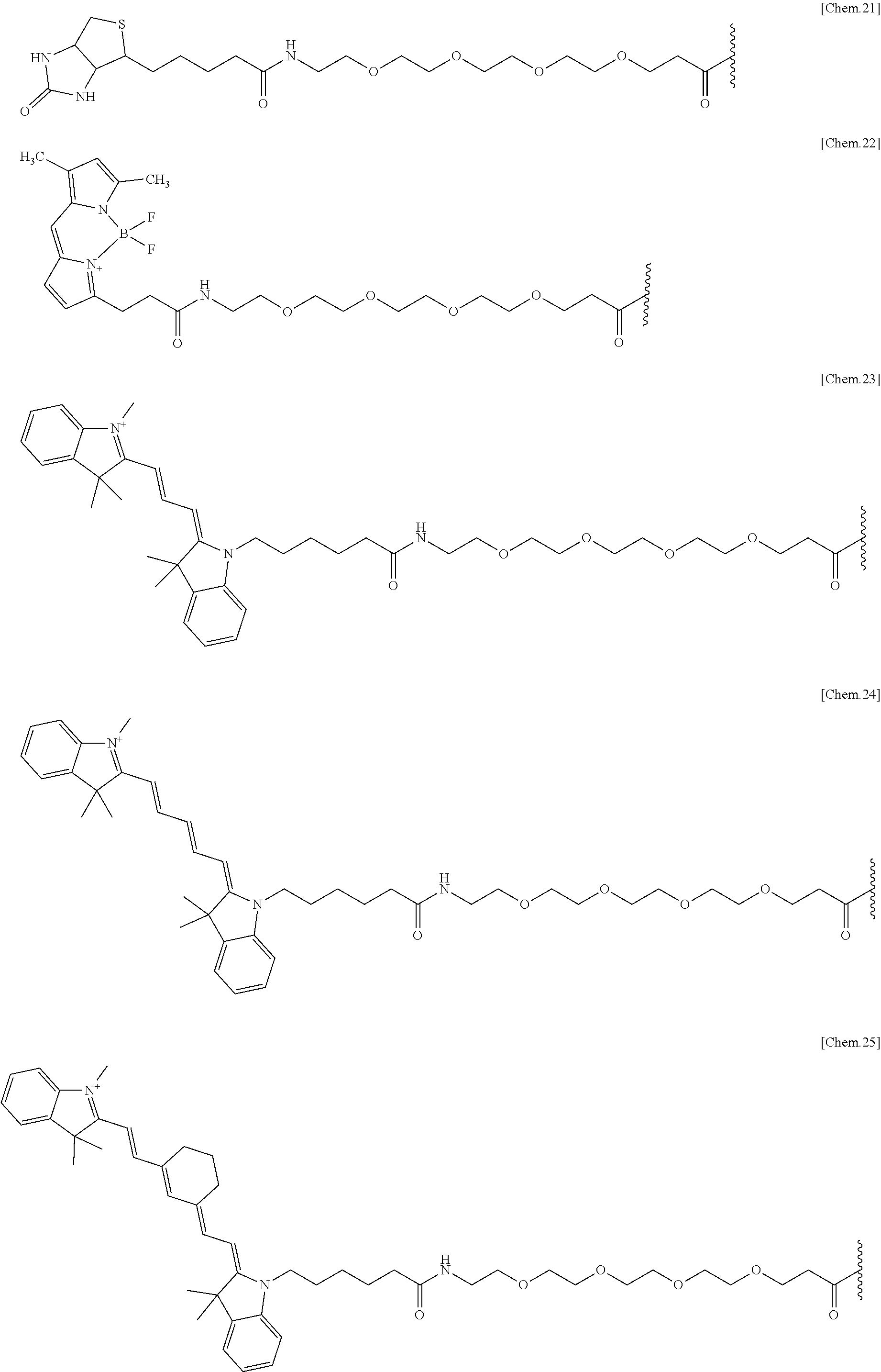

wherein in each of the above formulas, the curled line represents the point of connection to the remainder of the molecule.

Detectable Element:

In certain embodiments, the detectable element is selected from the group consisting of a fluorescent label, a biotin label, a radiolabel, a chelator (e.g., for a radiolabel), and a bioorthogonal ligation handle.

The detectable element, such as the fluorescent label, biotin label, radiolabel, chelator, or bioorthogonal ligation handle, can include a linker for incorporation into the compounds of the present invention (i.e., for attachment of the detectable element or label to the remainder of the molecule). Suitable linkers are known to those of skill in the art. Examples of linkers which can be used in the compounds of the present invention are described in WO 2012/118715 A2 (see page 18, lines 9-18), the contents of which are hereby included into the present disclosure. The linker can also include a polyethylene glycol (PEG) moiety, such as PEG-4, PEG-6 or PEG-8 for attachment to the remainder of the molecule.

A definition of the term “radiolabel” and examples of radiolabels which can be used in the compounds of the present invention are described in WO 2009/124265 A1 (see page 11, line 25 to page 13, line 3), the contents of which are hereby included into the present disclosure.

A definition of the term “chelator” and examples of chelators which can be used in the compounds of the present invention are described in WO 2009/124265 A1 (see page 10, line 26 to page 11, line 14), the contents of which are hereby included into the present disclosure.

A definition of the term “bioorthogonal ligation handle” and examples of bioorthogonal ligation handles which can be used in the compounds of the present invention and respective “click” reactions are described, e.g., in Martell et al., Applications of Copper-Catalyzed Click Chemistry in Activity-Based Protein Profiling, Molecules 2014, 19, 1378-1393, which is incorporated herein by reference. Adaptation of these methods to generate or modify compounds of the instant claims is within the skill in the art.

Bioorthogonal or click reactions for attachment of the secondary label include

A. the traceless Staudinger Ligation coupling azides with triarylphosphines to generate an amide linkage,

B. the tetrazine cycloaddition utilizing a 1,2,4,5-tetrazine and a strained diene (trans-cyclooctene),

C. the copper (I)-catalyzed azide-alkyne cycloaddition (CuAAC) reaction between an azide and a terminal alkyne to generate a 1,4-disubstituted 1,2,3-triazole, and

D. the copper-free variant of the azide-alkyne cycloaddition utilizing a strained alkyne to accelerate the reaction.

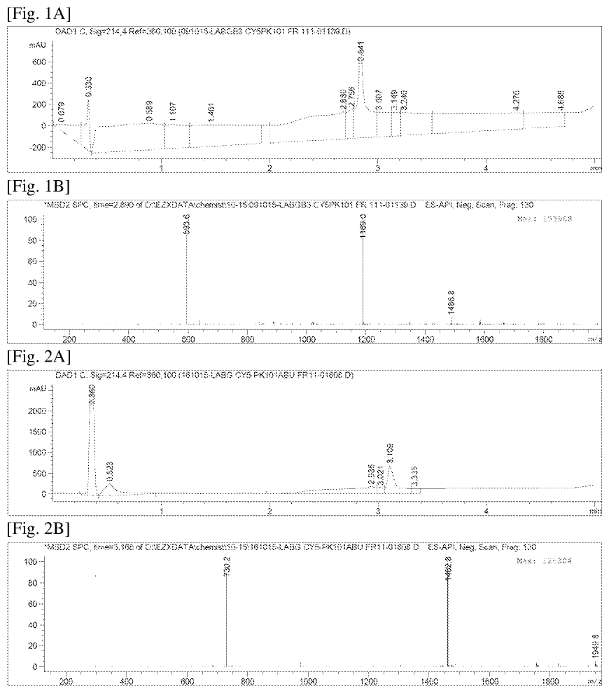

In this regard, reference is particularly made to FIG. 1B and FIG. 2 of Martell et al., Molecules 2014, 19, 1378-1393, the contents of which are hereby included into the present disclosure.

Thus, in certain embodiments, the bioorthogonal ligation handle comprises a functional group selected from the group consisting of an azide, a 1,2,4,5-tetrazine, and an alkyne (such as a terminal alkyne). These functional groups allow the attachment of a secondary label using one of the above bioorthogonal reactions (A) to (D).

In certain embodiments, the detectable element is a fluorescent label. As is known by those of skill in the art, fluorescent labels emit electromagnetic radiation, preferably visible light, when stimulated by the absorption of incident electromagnetic radiation. A wide variety of fluorescent labels, including labels having reactive moieties useful for coupling the label to reactive groups, such as, for example, amino groups, thiol groups and the like, are commercially available. See, e.g., The Molecular Probes (registered trademark) Handbook—A guide to Fluorescent Probes and Labeling technologies, which is hereby incorporated by reference in its entirety.

Examples of fluorescent labels which can be used in the compounds of the present invention are described in WO 2018/119476 A1 (see paragraphs [0084] to [0095]) and in WO 2012/118715 A2 (see page 15, line 18 to page 17, line 12, and page 18, line 19 to page 21, line 1), the contents of which are hereby included in the present disclosure. Such fluorescent labels can include a linker for incorporation into the compounds of the present invention, e.g., as described in WO 2012/118715 A2 (see page 18, lines 9-18), the contents of which are hereby included into the present disclosure.

In certain embodiments, the detectable element is a fluorescent label. In certain such embodiments, the fluorescent label is selected from the group consisting of a fluorescein, an Oregon green (a fluorinated derivative of fluorescein), a bora-diaza-indecene dye, a rhodamine dye (such as tetramethylrhodamine and carboxy tetramethyl rhodamine), a benzopyrillium dye, a coumarin dye, a cyanine label or a benzoindole label (such as indocyanine green).

Commercially available examples of such dyes include the BODIPY (registered trademark) dyes (bora-diaza-indecene dyes), dyes of the Alexa Fluor&reg; series (sulfonated rhodamines), dyes of the DyLight (registered trademark) series (having e.g. a sulfonated or unsulfonated coumarin, rhodamine, benzopyrilium, or cyanine as base structure), dyes of the IRDye (registered trademark) series, and cyanine (Cy) dyes (e.g. Cy2, Cy3, Cy3.5, Cy5, Cy5.5, Cy7, Cy7.5, sCy3, sCy5, and sCy7). Such cyanine labels can be purchased, e.g., from the companies Abcam, Tocris, GoldBio, ThermoFisher, Kerafast, Lumiprobe, AAT Bioquest or W&J Pharmachem.

In certain embodiments the fluorescent label is a cyanine label. In certain such embodiments the fluorescent label is a cyanine label selected from the group consisting of Cy2, Cy3, Cy3.5, Cy5, Cy5.5, Cy7, Cy7.5, sCy3, sCy5, and sCy7. In certain such embodiments the fluorescent label is Cy5 or sCy5. In certain embodiments the fluorescent label is sCy5.

In certain embodiments the fluorescent label is a cyanine label having a formula selected from the following group of formulas:

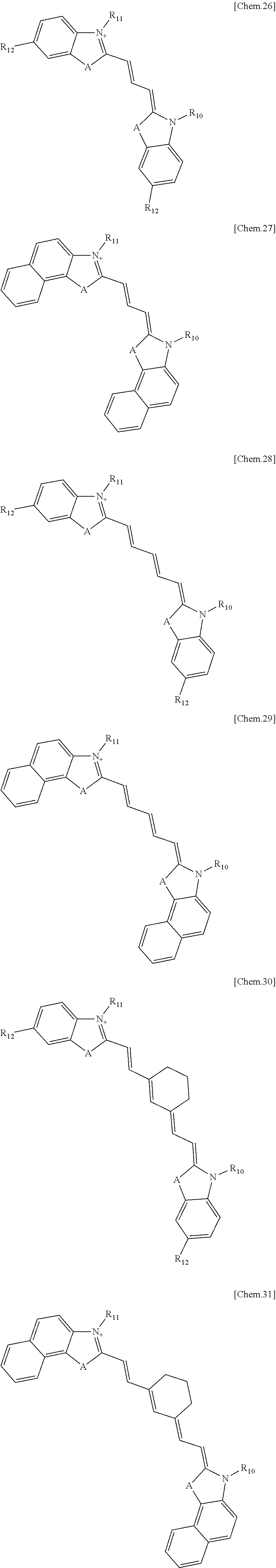

wherein in each of the above formulas,

A is selected from the group consisting of CH₂, C(CH₃)₂, C(C₂H₅)₂, NH, N(CH₃), N(C₂H₅), O, S, and Se; R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, 6, 7, or 8;

q is 2, 3, 4, 5, 6, 7, or 8;

r is 2, 3, 4, 5, 6, 7, or 8;

$ represents the point of connection to the nitrogen atom of the cyanine moiety; and & represents the point of connection to the remainder of the molecule;

R₁₁ is selected from the group consisting of (C₁-C₈)alkyl, and (C₆-C₁₀)aryl; and

R₁₂ is H or a sulfo group. In certain embodiments, R₁₀ is $-(CH₂)ₚ—C(═O)-&. In certain embodiments, R₁₂ is a sulfo group. In certain embodiments, p is 5, q is 5 and r is 4.

In certain embodiments wherein the fluorescent label is a cyanine label having one of the above formulas,

A is selected from the group consisting of CH₂, C(CH₃)₂, and C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is (C₁-C₈)alkyl; and

R₁₂ is H or a sulfo group. In certain embodiments, R₁₀ is $-(CH₂)ₚ—C(═O)-&. In certain embodiments, R₁₂ is a sulfo group. In certain embodiments, p is 5, q is 5 and r is 4.

In certain embodiments wherein the fluorescent label is a cyanine label having one of the above formulas,

A is C(CH₃)₂ or C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is methyl, ethyl or propyl; and

R₁₂ is H or a sulfo group. In certain embodiments, R₁₀ is $-(CH₂)ₚ—C(═O)-&. In certain embodiments, R₁₂ is a sulfo group. In certain embodiments, p is 5, q is 5 and r is 4.

In certain embodiments wherein the fluorescent label is a cyanine label having one of the above formulas,

A is C(CH₃)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 4, 5, or 6;

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H or a sulfo group. In certain embodiments, R₁₀ is $-(CH₂)ₚ—C(═O)-&. In certain embodiments, R₁₂ is a sulfo group. In certain embodiments, p is 5, q is 5 and r is 4.

In certain embodiments wherein the fluorescent label is a cyanine label having one of the above formulas,

A is C(CH₃)₂;

R₁₀ is $-(CH₂)ₚ—C(═O)-&; wherein

p is 4, 5, or 6; and

$ represents the point of connection to the nitrogen atom of the cyanine moiety; and

& represents the point of connection to the remainder of the molecule;

R₁₁ is methyl or ethyl; and

R₁₂ is a sulfo group. In certain such embodiments, p is 5.

In certain embodiments wherein the fluorescent label is a cyanine label having one of the above formulas,

A is C(CH₃)₂;

R₁₀ is $-(CH₂)_{q}—C(═O)—NH—[CH₂CH₂O]ᵣ—CH₂CH₂—C(═O)-&;

wherein

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H. In certain such embodiments, q is 5 and r is 4.

In certain embodiments, the fluorescent label is a cyanine label having a formula selected from the following group of formulas:

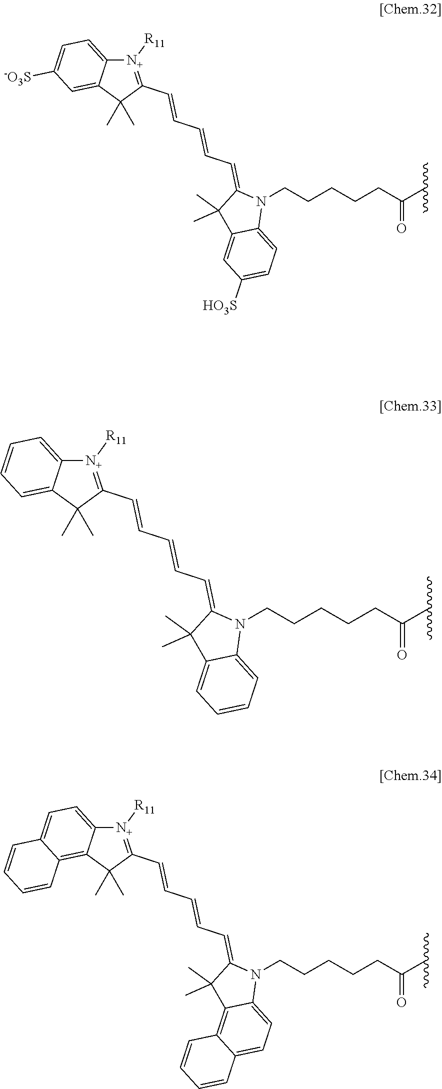

wherein in each of the above formulas,

the curled line represents the point of connection to the remainder of the molecule;

and R₁₁ is selected from the group consisting of (C₁-C₈)alkyl, and (C₆-C₁₀)aryl. In certain such embodiments, R₁₁ is (C₁-C₈)alkyl. In certain such embodiments, R₁₁ is methyl or ethyl.

In certain embodiments, the fluorescent label is a cyanine label having the formula

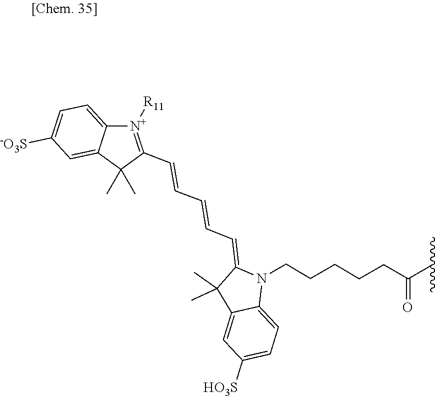

wherein the curled line represents the point of connection to the remainder of the molecule; and R₁₁ is methyl or ethyl.

Thus, in certain embodiments of the methods of detecting NE activity, methods of diagnosis, and in vitro methods of inhibiting NE as described herein, in step (2) the lysate is contacted with a compound of formula II

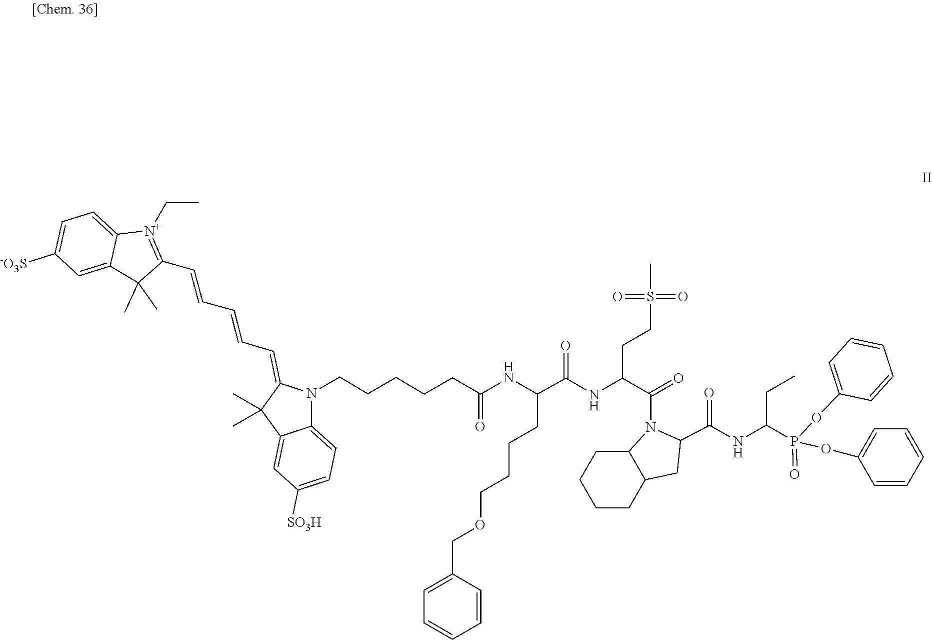

or a salt thereof.

In certain such embodiments, in step (2) the lysate is contacted with a compound of formula IIA

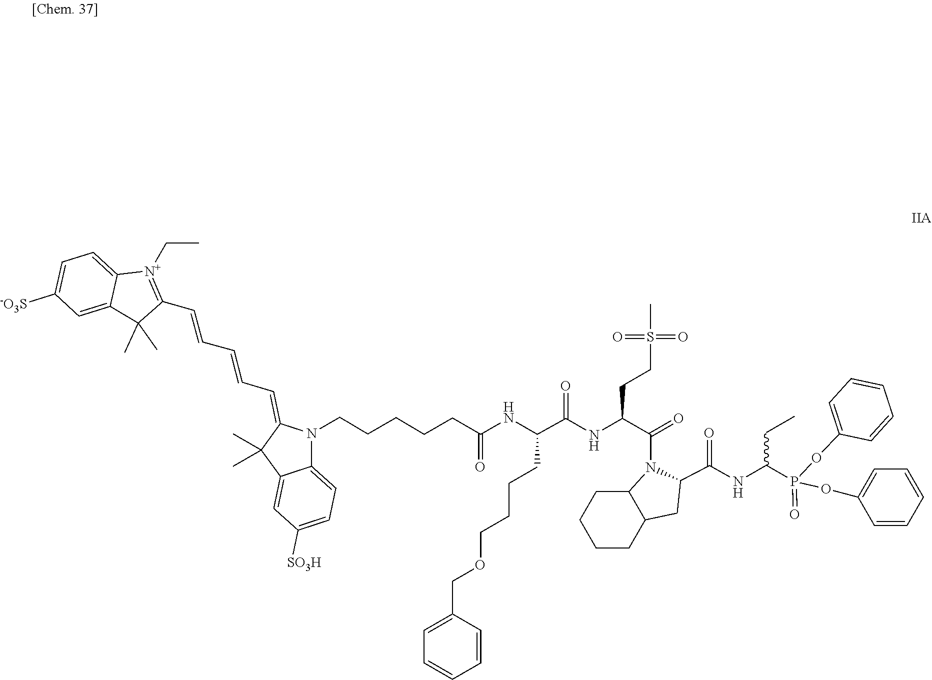

or a salt thereof.

In certain embodiments the present invention also relates to a compound of formula II

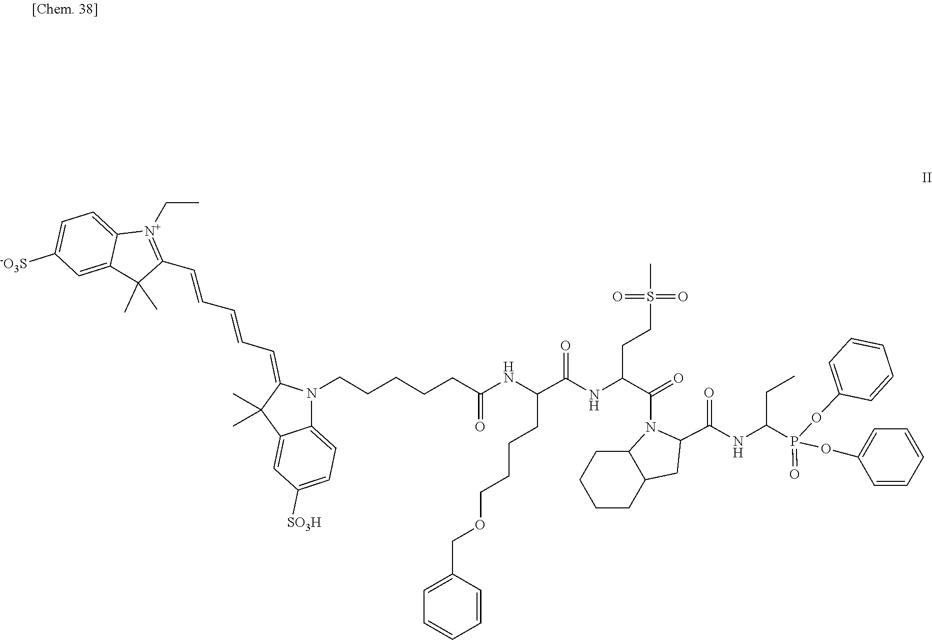

or a salt thereof, or to a compound of formula IIA

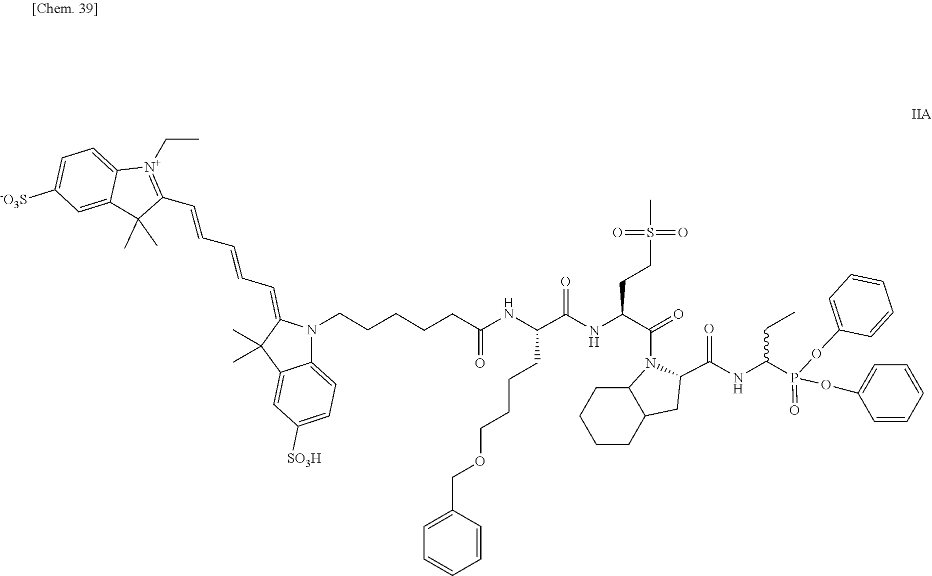

or a salt thereof.

In certain embodiments, the present invention relates to a composition comprising a compound (of formula I, IA, II or IIA) as described herein or a salt thereof, and an excipient.

### DETAILED DESCRIPTION OF THE PREFERRED EMBODIMENTS

The present invention is now more fully described with reference to the accompanying examples. It should be understood, however, that the following description is illustrative only and should not be taken in any way as a restriction of the invention.

### EXAMPLES

I. Synthesis and Characterization of Compounds

General Information

Fmoc amino acids were purchased from Chem-Impex and Novabiochem, coupling reagents were purchased from GL Biochem, and solvents and other reagents were purchased from Merck and used without further purification.

Resins were purchased from Chem-Impex.

Cy5-acid was purchased from Lumiprobe.

RP-HPLC purification of crude peptides was performed on an Agilent 1200 quaternary pump system, photodiode array detector (214 nm), employing a Phenomenex Axia column (Luna C8(2), 50×21.3 mm ID) eluting with a gradient of 5-100% of 0.1% TFA/acetonitrile in 0.1% aqueous TFA, over 60 minutes at a flow rate of 10 mL/min. Appropriate fractions collected were analyzed by LC-MS on an Agilent 1260SQ system, incorporating a photodiode array detector (214 nm) coupled directly to an API-ES quadrupole mass analyser. The combined fractions were freeze-dried for two days to give the purified peptides as TFA salts and their purity was >90% as estimated by reversed-phase HPLC carried out employing a Poroshell 120 EC-C18 3.0×50 mm 2.7-Micron eluting with a gradient of 5-100% acetonitrile in 0.1% aqueous formic acid, over 3.8 min and maintained to 100% acetonitrile until 5 min at a flow rate of 0.5 mL/min, detection was at 214 nm.

The compounds were confirmed as having the correct molecular weight by API-ES MS analysis. Mass spectra were acquired in negative ion mode with a scan range of 200-2000 m/z.

### Example 1: Synthesis of sulfoCy5-Nle(OBzl)-Met(O)₂-Oic-OH

Synthesis of the protected linear peptide (Cy5-Nle(OBzl)-Met(O)₂-Oic-OH) was carried out using manual peptide synthesis with standard Fmoc solid phase peptide chemistry. Synthesis was undertaken using Chlorotrityl chloride resin (loading 1.0 mmol/g from Chem-Impex) on a 0.2 mmol scale (0.3 g of resin). Coupling of the first amino acid was performed with Fmoc-Oic-OH (1.2 mol eq relative to resin loading) in dichloromethane (DCM) activated with 3 mol eq of diisopropylethylamine (DIPEA). This was carried out overnight at room temperature. The resin was then washed with DMF (3×5 mL×2 min each and then DCM 2×5 mL×2 min each) and then exposed to the deprotection solution 20% piperidine in DMF (3×5 mL×5 min each) and after the third deprotection step a positive bromophenol blue test resulted.

Coupling of subsequent Fmoc-amino acids was performed using the 1.5 mol eq. (relative to resin loading) of Fmoc amino acid, PyBOP (1H-Benzotriazol-1-yloxy)(tri-1-pyrrolidinyl)phosphonium hexafluorophosphate in DMF (5 mL/g of resin) with activation in situ, using 3 mol equiv of DIPEA. This was carried out for 1 h at room temperature (RT). At this stage the TNBS test was used to monitor peptide coupling providing a negative result. The resin was then washed with DMF (3×5 mL×2 min each and then DCM 2×5 mL×2 min each). The resin was then exposed to the deprotection solution 20% piperidine in DMF (3×5 mL×5 min each) and after the third deprotection step a positive TNBS test resulted. The resin was washed with DMF (3×5 mL×2 min each and then DCM 2×5 mL×2 min each) and the coupling process continued with the next Fmoc amino acid until the sequence was completed.

The final amino acid on the peptide resin was Fmoc deprotected with 20% piperidine in DMF (3×5 mL×5 min each) and then thoroughly washed with DMF then DCM. A portion of the resin (30 mg, 0.03 mmol) was suspended in 4:1 DMF:DMSO and sulfoCy5 acid (30 mg, 0.046 mmol) was added to the mixture followed by PyBOP (0.1 mmol) and finally DIPEA (0.6 mmol). The mixture was left for 24 h with intermittent agitation and then thoroughly washed with DMSO (until a colorless filtrate was obtained), followed by DMF, DCM, MeOH and finally Ether. The dried resin was taken up in 5 mL of HFIP (hexafluoroisopropanol):DCM:TIPS (v:v:v, 30:69:1) and left to stand for 2 h. The filtrate was filtered from the resin and the resin washed with HFIP until colorless. The combined filtrate and washings were concentrated to a residue (10.1 mg) and then purified by RP-HPLC providing 3 mg of the intermediate sulfoCy5-Nle(OBzl)-Met(O)₂-Oic-OH as a blue powder.

The compound was checked for purity by HPLC absorbance measurement at 214 nm

(FIG. 1A) and confirmed as having the correct molecular weight by API-ES analysis: m/z calculated; C₆₀H₇₉N₅O₁₄S₃ [M-H]⁻ 1189.5, [M-2H]²⁻ 593.7; observed: [M-H]⁻ 1189.0, [M-2H]²⁻ 593.6 (FIG. 1B).

### Example 2: Synthesis of PK105b

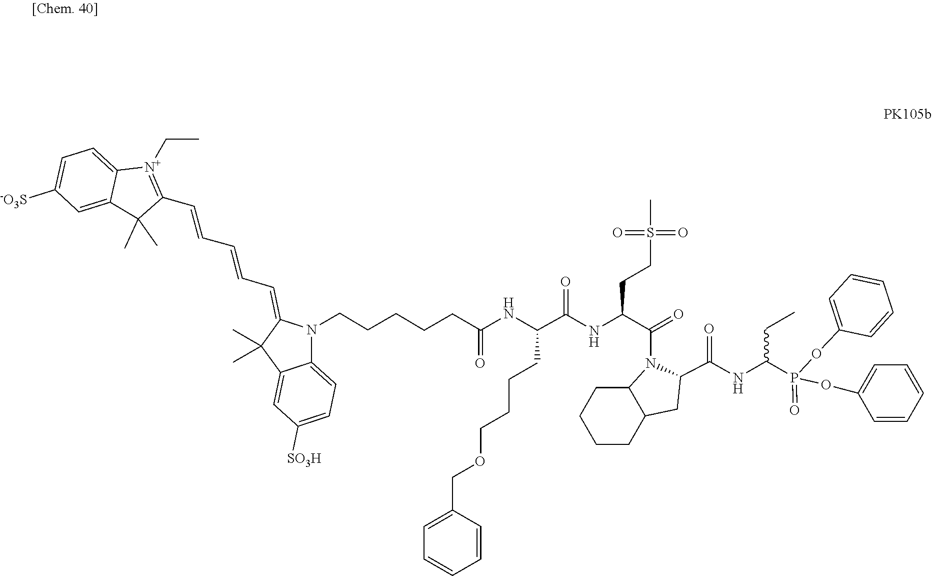

Cy5-Nle(OBzl)-Met(O)₂-Oic-OH from Example 1 (1 mg) was taken up in dry DMSO (50 μL) in an Eppendorf tube (1.5 mL) and to this mixture was added PyBOP (2 mol eq), Abu^{P}(OPh)₂.HBr (1.2 mol eq) followed by DIPEA (6 mol eq). The mixture was agitated for 24 h and then diluted in ACN (6 mL) and purified by RP-HPLC providing 0.7 mg of the final compound PK105b as a blue powder.

The compound was checked for purity by HPLC absorbance measurement at 214 nm (FIG. 2A) and confirmed as having the correct molecular weight by API-ES analysis: m/z calculated; C₇₅H₉₅N₆O₁₆PS₃ [M-H]⁻ 1462.8, [M-2H]²⁻ 730.3; observed: [M-H]⁻ 1462.8, [M-2H]²⁻ 730.2 (FIG. 2B).

II. Testing of Probes

General Information

Materials and Methods

Probe Synthesis and Characterization

Synthesis and characterization of PK105b was carried out as described in Examples 1 and 2. Synthesis and characterization of Cy5-V-DPP was carried out as described in Edgington-Mitchell et al., Bioorg. Med. Chem. Lett. (2017).

Mice

C57BL/6J mice were purchased from the Monash University in-house colony or the Bio21 in-house colony at the University of Melbourne. BALB/c nude mice were purchased from Charles River Laboratories. Unless otherwise specified, animal experiments were approved by the Animal Ethics Committee of Monash University in accordance with guidelines for the use of laboratory animals in research.

Recombinant Protease Labeling/Fluorescent SDS-PAGE

Recombinant proteases (500 ng) were diluted in 20 μl of phosphate-buffered saline (PBS): neutrophil elastase (Elastin Products Company), porcine pancreatic trypsin type II-S (beta trypsin; Sigma), and human proteinase-3 (Sigma). PK105b or Cy5-V-DPP (0, 0.1, 0.5 or 1 μM) was added from a 100×DMSO stock, and reaction was left to occur at 37° C. for 30 minutes. Proteins were solubilized in 4× sample buffer (40% glycerol, 200 mM Tris-Cl [pH 6.8], 8% SDS, 0.04% bromophenol blue, 5% beta-mercaptoethanol), boiled for five minutes and resolved on a 15% SDS-PAGE gel. Probe labeling was detected by scanning the gel for Cy5 fluorescence on a Typhoon 5 flatbed laser scanner (GE Healthcare). Detailed protocols for ABP application are available in Edgington and Bogyo Curr Protoc Chem Biol (2013).

Ex Vivo Tissue Labeling

Bone marrow was obtained by flushing tibias and femurs from healthy C57BL/6J mice with PBS. Cells were washed and resuspended in PBS prior to sonication on ice. Pancreata, colon tissues, mucosal biopsies, lungs, and tumors were lysed by sonication on ice in PBS (10 μl/mg tissue), and supernatants were cleared by centrifugation at 21 g for 10 min at 4° C. Total protein (60 μg, as measured by BCA assay, Pierce) was aliquoted in a total volume of 20 μl PBS, and probe labeling and SDS-PAGE was carried out as above.

Western Blotting

Fluorescent Gels were Transferred to Nitrocellulose Membranes and Blotted Using the Turbo Blot system (BioRad). Membranes were blocked using LiCor Odyssey blocking buffer diluted by 50% with PBS contained 0.05% Tween 20. Sheep anti-mouse neutrophil elastase/ELA2 (1:1000; R&D AF4517) was incubated overnight at 4° C. Secondary antibody (goat-IR800, 1:5000; LiCor) was incubated for one hour at room temperature. Binding was detected by scanning with the IRLong filter on a Typhoon 5 flatbed laser scanner (GE Healthcare).

Immunoprecipitation

PK105b-labeled lysates (boiled in sample buffer for five minutes) were divided into input and immunoprecipitation (IP) samples (100 μg each). The IP samples were diluted in 500 μl IP buffer (PBS [pH 7.4], 1 mM EDTA, 0.5% NP-40) along with 10 μl of one of the following antibodies: Sheep anti-neutrophil elastase/ELA2 (R&D AF4517); rabbit anti-PRSS3 (Trypsin 3; Abcam ab105123); rabbit anti-pancreatic elastase (Abcam ab21593). Protein A/G beads (40 μl slurry; Santa Cruz) were washed with IP buffer and then added to the sample. Tubes were rocked overnight at 4° C. Beads were washed four times with IP buffer and once with 0.9% sodium chloride. After the last wash, all buffer was removed and beads were boiled in 2× sample buffer (20 μl) for five minutes. Supernatants were then analyzed, alongside the input sample, by fluorescent SDS-PAGE as above.

Colitis Model

Colitis was induced in 10-week old male C57BL/6J mice by intracolonic infusion of picrylsulfonic acid solution (2,4,6-Trinitrobenzenesulfonic acid solution, TNBS; Sigma; 2.5 mg dissolved in 50% ethanol). Body weight and symptoms were recorded daily, and mice were humanely killed after three days. Upon colon extraction, luminal fluids were collected by flushing colons with PBS. Solids were removed by centrifugation and supernatant was concentrated using a 3-KDa cut-off centrifugal filter (Amgen). Pieces of proximal and distal colon were frozen for protease analysis or fixed in 4% paraformaldehyde overnight, paraffin embedded, sectioned, and stained with haematoxylin and eosin.

Human Mucosal Biopsies

Human mucosal biopsies were obtained from individuals during colonoscopy procedures at Hotel Dieu Hospital in Kingston, Ontario, Canada (Table 1). Patients were well-characterized individuals with active ulcerative colitis (UC) or healthy individuals undergoing routine colonoscopy for cancer screening. For UC patients, biopsies were obtained from sites of active inflammation. Written and verbal consent was obtained prior to enrolment and all protocols were approved by the Queen's University Human Ethics Committee. Fresh biopsies were washed in PBS and then snap frozen for protease analysis as above.

TABLE 1
Human patient data

| PT# | Symptoms | Medication | Pathology | Endoscopy |
|---|---|---|---|---|
| 1 | none | none | normal tissue | none |
| 2 | none | none | normal tissue | none |
| 3 | none | none | normal tissue | none |
| 4 | none | none | normal tissue | none |
| 5 | flare-up, | Steriods, | chronic inflammation, | pancolitis, Mayo 3 |
| | 15-20 bm/d | biologic | severe activity | distal,2 proximal |
| 6 | flare-up, | none | chronic inflammation, | pancolitis, |
| | 10 bm/d | | moderate to severe acticity | Mayo 2 |
| 7 | chronic active, | none | chronic inflammation, | proctitis, Mayo 1 |
| | 4 bm/d | | mild activity | |
| 8 | chronic active, | 5-ASA | chronic inflammation, | pancolitis, Mayo 3 |
| | 2-3 bm/d | | marked activity | distal, 2 promixal |
| 9 | new oneset, | none | chronic inflammation, | pancolitis, Mayo 2 |
| | 6-8 bm/d | | moderate activity | |
| 10 | chronic active, | Steriod enema, | chronic inflammation, | proctitis, Mayo 2 |
| | 12 bm/d* | 5-ASA | mild activity | |
| 11 | flare-up, | Imuran | acute, chronic inflammation, | ileocolitis, deep |
| | 6-8 bm/d | | deep ulcers | ulcers |
| 12 | flare-up, | 5-ASA | chronic inflammation, | colitis |
| | 4-5 bm/d | | severe activity | |
| 13 | flare-up, pain, | 5-ASA | normal tissue; chronic stricture | ileal stricture, |
| | +2 bm/d | | with no active inflammation | blind biopsies |
| 14 | flare-up, | none | chronic inflammation, | pancolitis, |
| | 2-5 bm/day | | moderate activity | Mayo 1-2 |

5-ASA = 5 aminosalicylic acid;
*mucous, but infrequent stool;
**suspected flare up initially but with further imaging dx with chronic stricture with no active inflammation;
bm = bowel movement; most were bloody

Mouse model of Legionella pneumophila infection

These experiments were performed under approval of the University of Melbourne

Animal Ethics Committee in accordance with guidelines for the use of laboratory animals in research. C57BL/6J mice were infected by intranasal inoculation with 2.5×10⁶ L. pneumophila 130b AflaA in 50 μL of PBS. Three days after infection, lungs were collected, snap frozen, and processed as above for labeling with PK105b.

Mouse Model of Oral Cancer

These experiments were approved by the Committee on Animal Research at New

York University in accordance with guidelines for the use of laboratory animals in research. Female BALB/c nude mice (6-8 weeks old, Charles River Laboratories) were injected in the left lateral tongue under anesthesia (3×10⁵ HSC-3 human oral squamous cell carcinoma cells suspended in 50 μl vehicle [1:2 mixture of DMEM and Matrigel; Becton Dickinson], or vehicle alone). After two weeks, the resulting xenografted tumors and vehicle-injected tongues were excised, snap frozen, and processed as above for labeling with PK105b.

Statistical Analysis

All experiments were performed with at least three biological replicates. Data are reported as means±SEM. Statistical significance was determined by comparing two groups using a Student's t test, and p values of less than 0.05 were considered significant.

### Example 3—Selectivity of PK105b Against Purified Serine Proteases

The reactivity of PK105b against recombinant human serine proteases was tested, and its potency was compared to Cy5-V-DPP. After a brief incubation of increasing amounts of PK105b (0, 0.1, 0.5, or 1 μM) with equal amounts of serine proteases (neutrophil elastase (NE), proteinase-3 (PR-3), or trypsin), the mixtures were resolved by SDS-PAGE and binding of Cy5-V-DPP or PK105b to the serine proteases was detected by in-gel fluorescence.

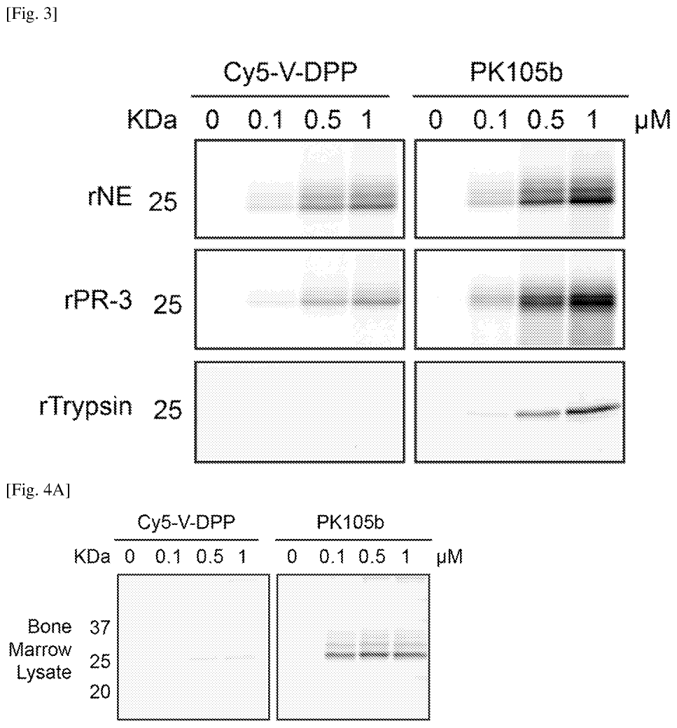

Both probes clearly labeled NE and PR-3 in a concentration-dependent manner (FIG. 3A), though PK105b was more potent than Cy5-V-DPP. PK105b also labeled trypsin, another serine protease, while trypsin binding by Cy5-V-DPP was negligible (FIG. 3A, bottom panels).

Example 4—Selectivity Profile of PK105b in Tissue Lysates

The ability of PK105b to detect serine protease activity in tissue lysates was tested and compared to Cy5-V-DPP.

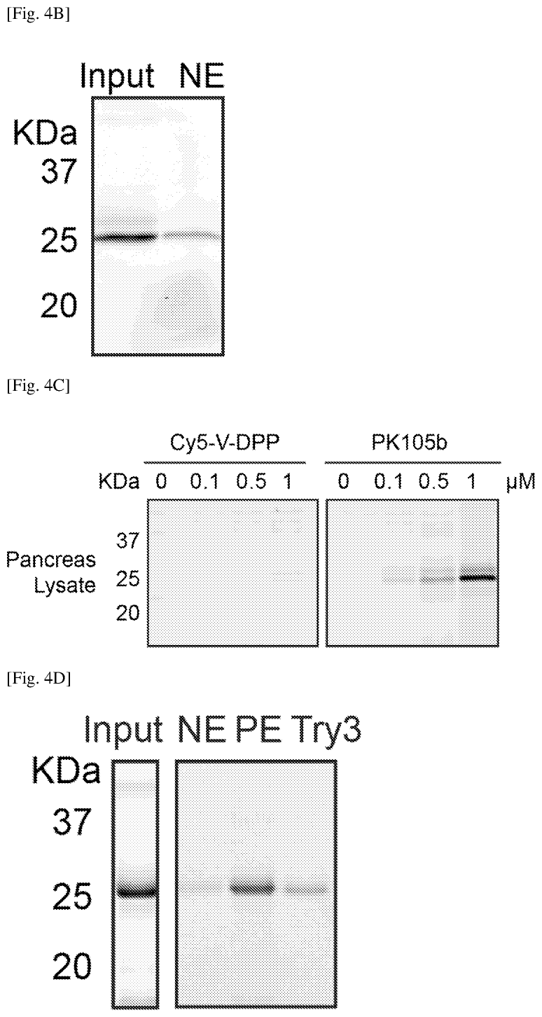

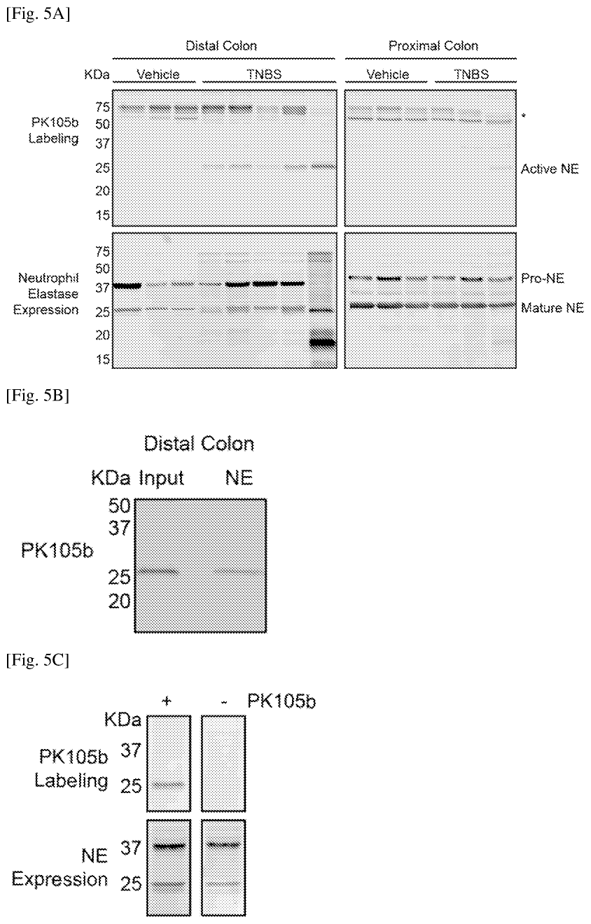

PK105b labeled multiple bands in lysates prepared from mouse bone marrow at 0.1, 0.5 and 1 μM, and it exhibited greater potency than Cy5-V-DPP (FIG. 4A). Of these bands, the 25-KDa protein was confirmed to be NE by immunoprecipitation with an NE-specific antibody (FIG. 4B).

The reactivity of Cy5-V-DPP and PK105b in lysates prepared from mouse pancreas, a tissue rich in serine proteases, was also examined. Here, PK105b strongly labeled 25-KDa proteins at 0.1, 0.5 and 1 &micro;M, but this was not observed with Cy5-V-DPP (FIG. 4C). Immunoprecipitation of PK105b-labeled mouse pancreas lysates revealed that the targets consisted of a combination of NE, pancreatic elastase (PE), and trypsin 3 (Try3, also known as PRSS3 or mesotrypsin; FIG. 4D).

### Example 5—Application of PK105b to Measure NE Activation in Experimental Colitis

PK105b was applied to investigate NE application during acute experimental colitis induced by trinitrobenzenesulfonate (TNBS). Mice in which experimental colitis was induced exhibited loose stools, delayed defecation, weight loss, and colon shortening. Damage to the mucosa was observed by histological evaluation, as well as edema and inflammatory infiltrate. Colon lysates were analyzed for NE activation by PK105b labeling and measurement of in-gel fluorescence. In the distal region of inflamed colons, which is most affected in the TNBS model, clear labeling of a single protein at 25-KDa was observed (FIG. 5A, top row, left panel). This band was virtually absent in distal colons of healthy mice that received vehicle instead of TNBS, as well as more proximal regions of healthy and inflamed colons (FIG. 5A, top row). The identity of the band was confirmed to be NE by immunoprecipitation with an NE-specific antibody (FIG. 5B).

The fluorescent gels were transferred to nitrocellulose membranes in order to immunoblot the samples for total NE expression. In healthy distal colons, a 37-KDa proform of NE as well as a 25-KD mature form were observed (FIG. 5A, bottom row, left panel). In the TNBS-treated colons, the 25-KDa band appeared as a doublet. Only the lower species was labeled by PK105b. To verify that appearance of this smaller NE species was not an artefact of probe labeling, inflamed distal colon samples were immunoblotted in the presence and absence of PK105b. The smaller species was detected regardless of the presence of PK105b (FIG. 5C). Furthermore, the smaller species was not detected in the proximal colon of TNBS-treated mice (FIG. 5A, right column of panels). Taken together, these data suggest that NE is subject to trimming in inflamed regions of the colon that permits its activation and thus its reaction with the PK105b probe.

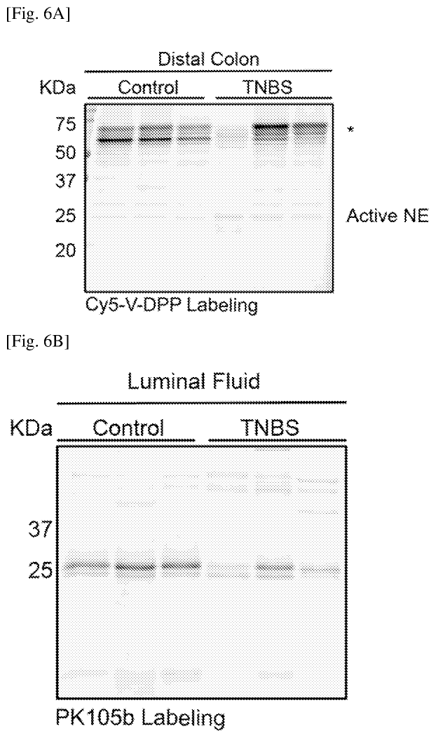

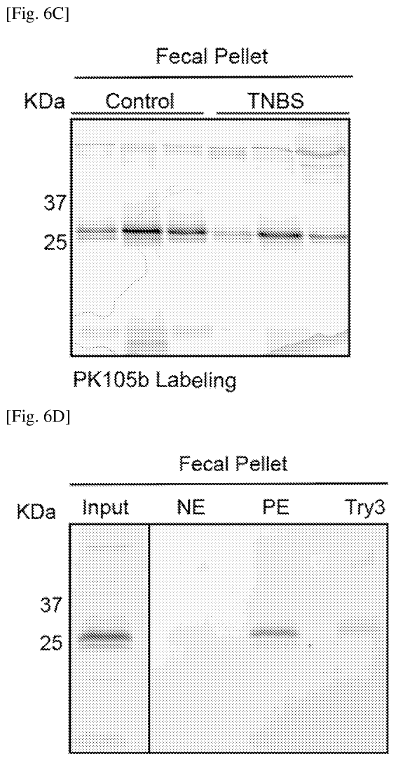

For comparison, probe Cy5-V-DPP was also tested in distal colon lysates. Labeling of the 25-KDa species was barely distinguishable from the background (FIG. 6A). Thus, PK105b is clearly superior to Cy5-V-DPP for its ability to detect NE activity in tissue lysates. Both probes exhibit binding to several species in the 50-75-KDa range (FIGS. 5A, 6A).

Furthermore, secreted serine proteases found in the lumen of the colon (either luminal flush or in fecal pellets) were also tested with PK105b (FIG. 6B-C). In both samples, two labeled serine proteases at 25 kDa were observed. Immunoprecipitation confirmed low levels of NE in these samples, with pancreatic elastase and trypsin 3 being the predominant species (FIG. 6D). Nonetheless, NE activity could be clearly delineated by PK105b in lysates from colon tissues.

### Example 6—Application of PK105b to Measure NE Activation in Mucosal Biopsies from Inflammatory Bowel Disease (IBD) Patients

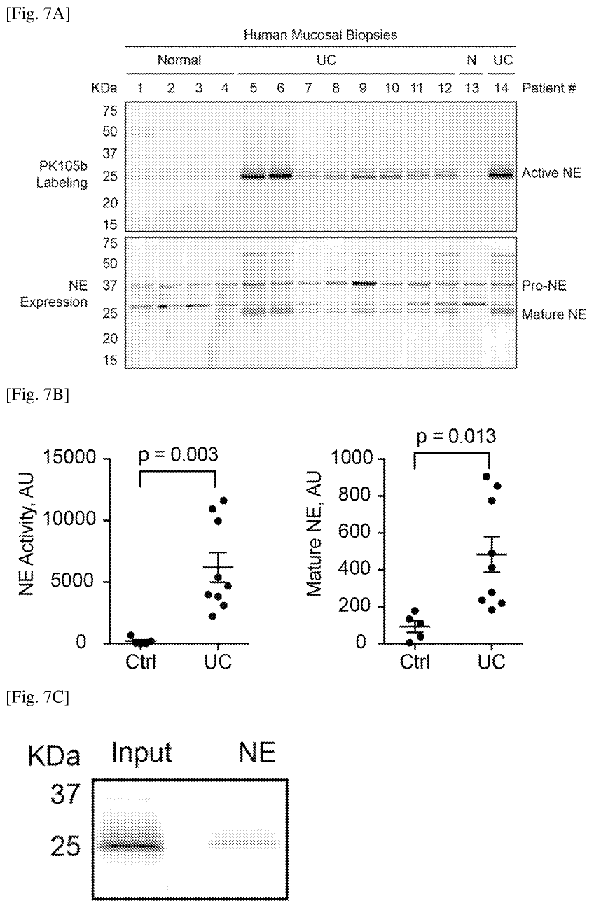

To translate the above findings in mouse colitis to patients, PK105b labeling in human colon mucosal biopsies was examined. As in mice, a significant increase in labeling in samples from patients with active ulcerative colitis (UC) was observed compared healthy individuals brought in for routine colonoscopy screening (FIGS. 7A top panel, 7B). In contrast to mice, where a single 25-KDa species labeled by PK105b was observed, three species were labeled in human mucosal lysates, with the smallest form having the most activity. The banding pattern resembled that which was observed with recombinant human NE (FIGS. 3A-B and Schultz-Fincke et al, ACS Med Chem Lett (2018), Dau et al, Nat Comm (2015)). These bands were confirmed to be NE by immunoprecipitation with an NE-specific antibody (FIG. 7C).

Furthermore, when the same samples were immunoblotted for total NE expression, the pro and mature forms of NE in the healthy tissue at 37 and 25 kDa, respectively, were observed (FIG. 7A, bottom panel). UC tissues, however, displayed an additional doublet that was smaller than the 25-KDa species. The most active species, as indicated by PK105b labeling, corresponded to these smaller species. Thus, as we observed in mouse colitis, NE undergoes differential trimming during human UC that permits its activation and binding to PK105b.

### Example 7—Application of PK105b to Measure NE Activation in Legionella pneumophila Infection

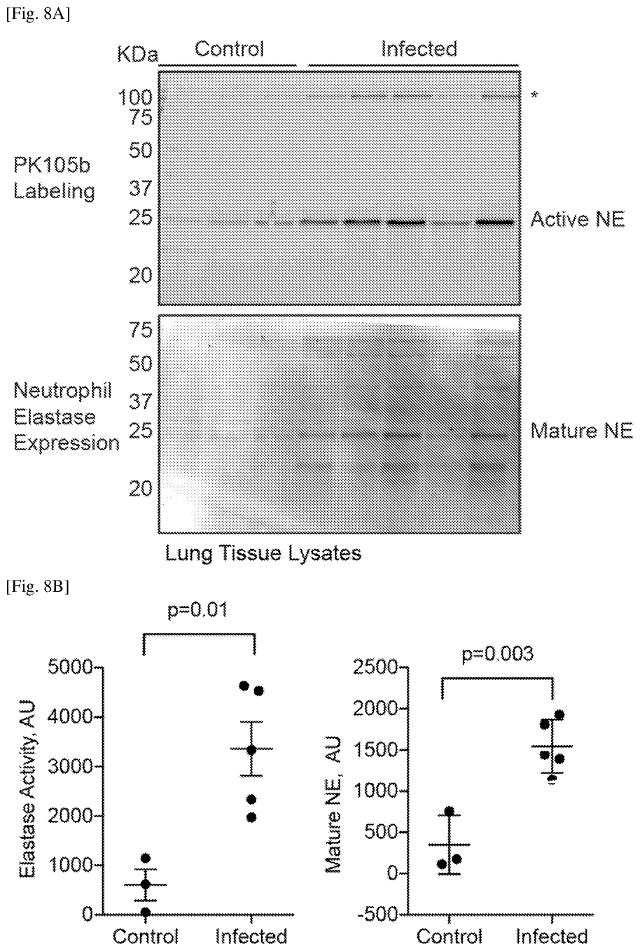

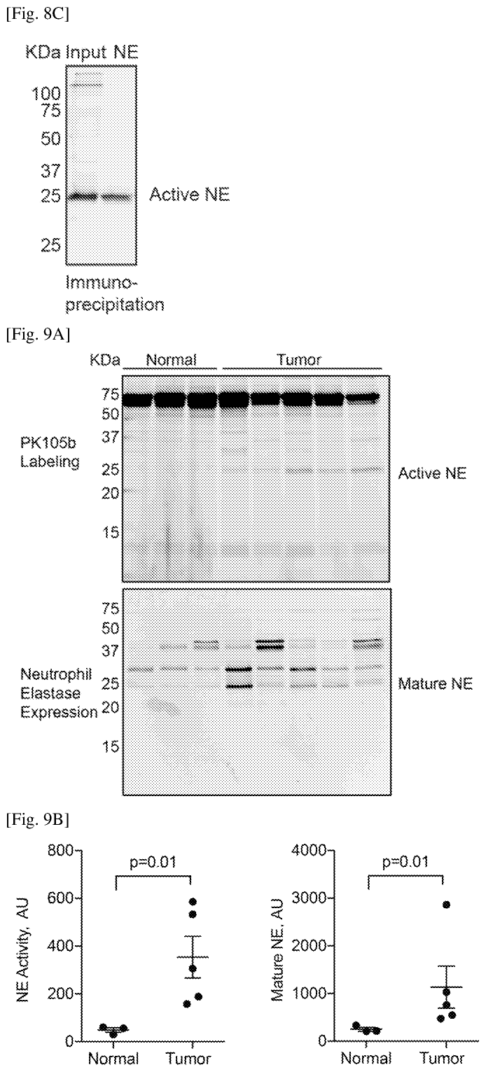

The effectiveness of PK105b to measure NE activation during infection was examined in a mouse model of Legionella pneumophila infection. PK105b labeling was significantly increased in lysates prepared from infected lung tissues compared to control lungs (FIGS. 8A top panel, 8B). The identity of the major 25-KDa species was confirmed to be NE by immunoblotting (FIG. 8A bottom panel) and immunoprecipitation (FIG. 8C) with an NE-specific antibody.

### Example 8—Application of PK105b to Measure NE Activation in Oral Cancer

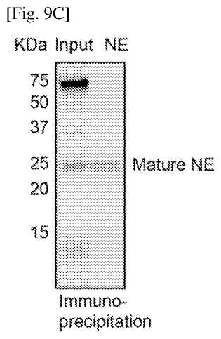

To determine the utility of PK105b to detect NE activation in a cancer setting, a mouse xenograft model of oral squamous cell carcinoma, in which human cancer cells (HSC-3) were injected into the tongue, was utilized. In this context, we observed clear labeling of a 25-KDa species in tumor tissues, but not normal tongue tissues (FIGS. 9A top panel, 9B). This species coincided with the size of mature NE as determined by immunoblot (FIG. 9A bottom panel) and also immunoprecipitated (FIG. 9C) with an NE-specific antibody. Several other unidentified high-molecular weight species were abundantly labeled by PK105b in these lysates.

The present examples, methods, procedures, specific compounds and molecules are meant to exemplify and illustrate the invention and should in no way be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but would be understood by workers in the field. Such patents or publications are hereby incorporated by reference to the same extent as if each was specifically and individually incorporated by reference and for the purpose of describing and enabling the method or material referred to.

### REFERENCES

Dau T, Sarker R S J, Yildirim A O, Eickelberg O, Jenne D E. Autoprocessing of neutrophil elastase near its active site reduces the efficiency of natural and synthetic elastase inhibitors. Nat Comm 6: 1-8, 2015.
Demkow U, van Overveld F. Role of elastases in the pathogenesis of chronic obstructive pulmonary disease: implications for treatment. Eur J Med Res 2:27-35, 2010.
Deu, E., Verdoes, M., and Bogyo, M., New approaches for dissecting protease functions to improve probe development and drug discovery. Nat. Struct. Mol. Biol. 19, 9-16, 2012.
Edgington L E, Bogyo M. In vivo imaging and biochemical characterization of protease function using fluorescent activity-based probes. Curr Protoc Chem Biol 5: 25-44, 2013.
Edgington L E, Verdoes M, Bogyo M. Functional imaging of proteases: recent advances in the design and application of substrate-based and activity-based probes. Curr Op Chem Biol 15: 798-805, 2011.
Edgington-Mitchell L E, Barlow N, Aurelio L, Samha A, Szabo M, Graham B, Bunnett N. Fluorescent diphenylphosphonate-based probes for detection of serine protease activity during inflammation. Bioorganic Med Chem Lett 27: 254-260, 2017.
Edgington-Mitchell L E. Pathophysiological roles of proteases in gastrointestinal disease. Am J Physiol Gastrointest Liver Physiol 310: G234-239, 2015.
Gecse K, Roka R, Ferrier L, Leveque M, Eutamene H, Cartier C, Ait-Belgnaoui A, Rosztoczy A, Izbeki F, Fioramonti J, Wittmann T, Bueno L. Increased faecal serine protease activity in diarrhoeic IBS patients: a colonic lumenal factor impairing colonic permeability and sensitivity. Gut 57: 591-599, 2008.
Jimenez-Vargas N N, Pattison L A, Zhao P, Lieu T, Latorre R, Jensen D D, Castro J, Aurelio L, Le G T, Flynn B, Herenbrink C K, Yeatman H R, Edgington-Mitchell L, Porter C J H, Halls M L, Canals M, Veldhuis N A, Poole D P, McLean P, Hicks G A, Scheff N, Chen E, Bhattacharya A, Schmidt B L, Brierley S M, Vanner S J, Bunnett N W. Protease-activated receptor-2 in endosomes signals persistent pain of irritable bowel syndrome. Proc Natl Acad Sci 115: E7438-E7447, 2018.
Kasperkiewicz P, Altman Y, D'Angelo M, Salvesen G S, Drag M. Toolbox of Fluorescent Probes for Parallel Imaging Reveals Uneven Location of Serine Proteases in Neutrophils. J Am Chem Soc 139: 10115-10125, 2017.
Kasperkiewicz P, Poreba M, Groborz K, Drag M. Emerging challenges in the design of selective protease substrates, inhibitors and activity-based probes for indistinguishable proteases. FEBS J 284: 1518-1539, 2017.
Kasperkiewicz P, Poreba M, Snipas S J, Parker H, Winterbourn C C, Salvesen G S, Drag M. Design of ultrasensitive probes for human neutrophil elastase through hybrid combinatorial substrate library profiling. Proc Natl Acad Sci 111: 2518-2523, 2014.
Kobayashi S D, Voyich J M, Burlak C, DeLeo F R. Neutrophils in the innate immune response. Arch Immunol Ther Exp 53:505-517, 2005.
Korkmaz B, Horwitz M S, Jenne D E, Gauthier F. Neutrophil elastase, proteinase 3, and cathepsin G as therapeutic targets in human diseases. Pharmacol Rev 62: 726-759, 2010.
Kuno Y, Ina K, Nishiwaki T, Tsuzuki T, Shimada M, Imada A, Nishio Y, Nobata K, Suzuki T, Ando T, Hibi K, Nakao A, Yokoyama T, Yokoyama Y, Kusugami K. Possible involvement of neutrophil elastase in impaired mucosal repair in patients with ulcerative colitis. J Gastroenterol 37: 22-32, 2002.
Lechtenberg B C, Kasperkiewicz P, Robinson H, Drag M, Riedl S J. The Elastase-PK101 Structure: Mechanism of an Ultrasensitive Activity-based Probe Revealed. ACS Chem Biol 10: 945-951, 2015.
Lerman I, Hammes S R. Neutrophil elastase in the tumor microenvironment. Steroids 133:96-101, 2018.
Lieu T, Savage E, Zhao P, Edgington-Mitchell L, Barlow N, Bron R, Poole D P, McLean P, Lohman R J, Fairlie D P, Bunnett N W. Antagonism of the proinflammatory and pronociceptive actions of canonical and biased agonists of protease-activated receptor-2. Brit J Pharmacol 173: 2752-2765, 2016.
Martell J, Weerapana E. Applications of Copper-Catalyzed Click Chemistry in Activity-Based Protein Profiling. Molecules 19:1378-1393, 2014.
Morohoshi Y, Matsuoka K, Chinen H, Kamada N, Sato T, Hisamatsu T, Okamoto S, Inoue N, Takaishi H, Ogata H, Iwao Y, Hibi T. Inhibition of neutrophil elastase prevents the development of murine dextran sulfate sodium-induced colitis. J Gastroenterol 41: 318-324, 2006.
Motta J P, Bermudez-Humaran L G, Deraison C, Martin L, Rolland C, Rousset P, Boue J, Dietrich G, Chapman K, Kharrat P, Vinel J P, Alric L, Mas E, Sallenave J M, Langella P, Vergnolle N. Food-Grade Bacteria Expressing Elafin Protect Against Inflammation and Restore Colon Homeostasis. Sci Trans Med 4: 158ra144-158ra144, 2012.
Motta J P, Magne L, Descamps D, Rolland C, Dale C S, Rousset P, Martin L, cenac N, Balloy V, Huerre M, Frohlich L F, Jenne D, Wartelle J, Belaaouaj A, Mas E, Vinel J P, Alric L, Chignard M, Vergnolle N, Sallenave J M. Modifying the Protease, Antiprotease Pattern by Elafin Overexpression Protects Mice from Colitis. Gastroenterol 140: 1272-1282, 2011.
Narita Y, Naoki K, Horiuchi N, Hida N, Okamoto H, Kunikane H, Watanabe K. [A case of legionella pneumonia associated with acute respiratory distress syndrome (ARDS) and acute renal failure treated with methylprednisolone and sivelestat][Article in Japanese]. Nihon Kokyuki Gakkai Zasshi 45:413-8, 2007.
Polverino E, Rosales-Mayor E, Dale G E, Dembowsky K, Tones A. The Role of Neutrophil Elastase Inhibitors in Lung Diseases. Chest 152:249-262, 2017.
Rawlings N D, and Salvesen G. Handbook of Proteolytic Enzymes, Third edition, Academic Press, Waltham, Mass., 2012.
Reeves E P, Lu H, Jacobs H L, Messina C G, Bolsover S, Gabella G, Potma E O, Warley A, Roes J, Segal A W. Killing activity of neutrophils is mediated through activation of proteases by K+ flux. Nature 416:291-297, 2002.
Schulz-Fincke A-C, Blaut M, Braune A, G&uuml; tschow M. A BODIPY-Tagged Phosphono Peptide as Activity-Based Probe for Human Leukocyte Elastase. ACS Med Chem Lett 9: 345-350, 2018.
Uchiyama K, Naito Y, Takagi T, Mizushima K, Hirai Y, Hayashi N, Harusato A, Inoue K, Fukumoto K, Yamada S, Handa O, Ishikawa T, Yagi N, Kokura S, Yoshikawa T. Serpin B1 protects colonic epithelial cell via blockage of neutrophil elastase activity and its expression is enhanced in patients with ulcerative colitis. Am J Physiol Gastrointest Liver Physiol 302: G1163-G1170, 2012.
Willems L I, van der Linden W A, Li N, Li K Y, Liu N, Hoogendoorn S, van der Marel G A, Florea B I, Overkleeft H S. Bioorthogonal chemistry: Applications in activity-based protein profiling. Acc Chem Res 44:718-729, 2011.
Zhao P, Lieu T, Barlow N, Sostegni S, Haerteis S, Korbmacher C, Liedtke W, Jimenez-Vargas N N, Vanner S J, Bunnett N W. Neutrophil Elastase Activates Protease-activated Receptor-2 (PAR2) and Transient Receptor Potential Vanilloid 4 (TRPV4) to Cause Inflammation and Pain. J Biol Chem 290: 13875-13887, 2015.

Further embodiments of the invention concern:

1. A method of detecting neutrophil elastase (NE) activity in a tissue sample lysate, comprising

(1) preparing the lysate from a tissue sample obtained from a subject,

(2) contacting the lysate with a compound of formula I

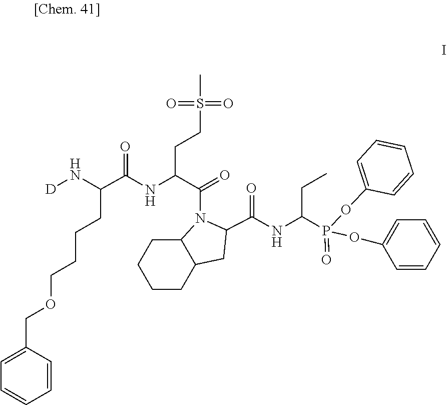

or a salt thereof,

wherein D is a detectable element,

(3) subsequently subjecting at least an aliquot of the lysate of step (2) to gel electrophoresis; and thereafter

(4) measuring a detectable signal.

2. The method of item 1, further comprising after step (3) a step

(5) immunoblotting with an anti-NE antibody.

3. The method of item 1 or 2, wherein additionally the following steps are performed:

(3a) immunoprecipitating the compound of formula I in a separate aliquot of the lysate of step (2) using an antibody specific for the compound of formula I or a part thereof,

(4a) subsequently analyzing co-precipitated material.

4. The method of item 3, wherein the analysis of step (4a) comprises
gel electrophoresis and subsequent immunoblot using an anti-NE antibody, or
protein sequencing,

and preferably comprises gel electrophoresis and subsequent immunoblot using an anti-NE antibody.

5. The method of any one of the preceding items, wherein prior to step (2), an aliquot of the lysate of step (1) is pretreated with a specific NE inhibitor, and wherein the pretreated aliquot is subsequently processed analogously to the not pretreated lysate of step (1).

6. The method of any one of the preceding items, wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy.

7. The method of item 6, wherein the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

8. The method of any one of the preceding items, wherein the subject is a human subject.

9. The method of any one of the preceding items, wherein an activated form of NE that is a trimmed form of mature NE is detected.

10. The method of item 9, wherein the tissue sample is selected from the group consisting of an oral biopsy, an esophagus sample, a stomach sample, a small intestine sample, a colon sample, a proximal colon sample, a distal colon sample, a rectal sample, a fecal sample, and a mucosal biopsy.

11. The method of item 10, wherein the tissue sample is a mucosal biopsy selected from the group consisting of an oral mucosal biopsy, an esophagus mucosal biopsy, a small intestine mucosal biopsy, a colon mucosal biopsy, and a rectal mucosal biopsy.

12. A method of diagnosing a disease associated with NE activity in a subject comprising

(1) preparing a lysate from a tissue sample obtained from the subject,

(2) contacting the lysate with a compound of formula I

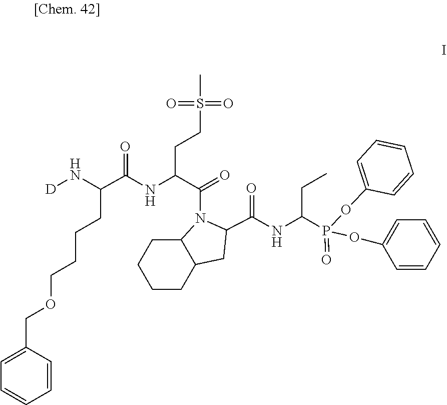

or a salt thereof,

wherein D is a detectable element,

(3) subsequently subjecting the lysate to gel electrophoresis; and thereafter

(4) measuring a detectable signal.

13. The method of item 12, wherein the disease associated with NE activity is selected from the group consisting of a celiac disease, a gastrointestinal motility disorder, pain, itch, a skin disorder, diet-induced obesity, a metabolic disorder, asthma, rheumatoid arthritis, periodontitis, an inflammatory GI disorder, a functional GI disorder, a cancer, a fibrotic disease, metabolic dysfunction, a neurological disease, a chronic obstructive pulmonary disease (COPD), and an infection.

14. The method of item 12, wherein the disease associated with NE activity is selected from the group consisting of an inflammatory bowel disease, an infection, a chronic obstructive pulmonary disease, and a cancer.

15. The method of any one of items 12 to 14, further comprising after step (3) a step (5) immunoblotting with an anti-NE antibody.

16. The method of any one of items 12 to 15, wherein additionally the following steps are performed:

(3a) immunoprecipitating the compound of formula I in a separate aliquot of the lysate of step (2) using an antibody specific for the compound of formula I or a part thereof,

(4a) subsequently analyzing co-precipitated material.

17. The method of item 16, wherein the analysis of step (4a) comprises
gel electrophoresis and subsequent immunoblot using an anti-NE antibody, or
protein sequencing,

and preferably comprises gel electrophoresis and subsequent immunoblot using an anti-NE antibody.

18. The method of any one of items 12 to 17, wherein prior to step (2), an aliquot of the lysate of step (1) is pretreated with a specific NE inhibitor, and wherein the pretreated aliquot is subsequently processed analogously to the not pretreated lysate of step (1).

19. The method of any one of items 12 to 18, wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy.

20. The method of item 19, wherein the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

21. The method of any one of items 12 to 20, wherein the subject is a human subject.

22. The method of any one of items 12 to 21, wherein the method is for diagnosing an inflammatory bowel disease.

23. The method of item 22, wherein an activated form of NE that is a trimmed form of mature NE is detected.

24. The method of item 23, wherein the subject is diagnosed as having an inflammatory bowel disease if the activated form of NE is detected.

25. The method of any one of items 22 to 24, wherein the tissue sample is selected from the group consisting of an oral biopsy, an esophagus sample, a stomach sample, a small intestine sample, a colon sample, a proximal colon sample, a distal colon sample, a rectal sample, a fecal sample, and a mucosal biopsy.

26. The method of item 25, wherein the tissue sample is a mucosal biopsy selected from the group consisting of an oral mucosal biopsy, an esophagus mucosal biopsy, a small intestine mucosal biopsy, a colon mucosal biopsy, and a rectal mucosal biopsy.

27. The method of any one of items 12 to 26, wherein the inflammatory bowel disease is selected from the group consisting of acute colitis, ulcerative colitis, Crohn's disease, microscopic colitis, diversion colitis, Behcet's disease, immuno-oncology colitis, chemotherapy/radiation colitis, Graft versus Host Disease colitis, collagenous colitis, lymphocytic colitis, and indeterminate colitis and pouchitis.

28. The method of any one of items 12 to 27, wherein the inflammatory bowel disease is ulcerative colitis.

29. The method of any one of items 12 to 27, wherein the inflammatory bowel disease is Crohn's disease.

30. The method of any one of items 12 to 21, wherein the method is for diagnosing an infection.

31. The method of item 30, wherein the infection is selected from the group consisting of a bacterial infection and a fungal infection.

32. The method of item 30 or 31, wherein the tissue sample is a sample from an infected tissue.

33. The method of any one of items 30 to 32, wherein the infection is an infection of the lung.

34. The method of item 33, wherein the infection of the lung is a bacterial infection.

35. The method of item 34, wherein the bacterial infection is an infection with Legionella.

36. The method of any one of items 33 to 35, wherein the tissue sample is selected from the group consisting of a lung sample, a lung mucosal biopsy or a sputum sample.

37. The method of any one of items 12 to 21, wherein the method is for diagnosing a cancer.

38. The method of item 37, wherein the tissue sample is selected from the group consisting of a tumor sample, an oral biopsy, an oral mucosal biopsy, a breast biopsy, a prostate biopsy, a colon biopsy, a colon mucosal biopsy, a rectal biopsy, a rectal mucosal biopsy, a lung biopsy, a lung mucosal biopsy, and a sputum sample.

39. The method of item 37 or 38, wherein the cancer is selected from the group consisting of an oral cancer, a breast cancer, a prostate cancer, a colorectal cancer, and a lung cancer.

40. The method of any one of item 37 to 39, wherein the cancer is an oral cancer and the oral cancer is a squamous cell carcinoma.

41. The method of any one of items 12 to 21, wherein the method is for diagnosing a chronic obstructive pulmonary disease.

42. The method of item 41, wherein the tissue sample is selected from the group consisting of a lung sample, a lung mucosal biopsy, and a sputum sample.

43. An in vitro method of inhibiting NE, comprising

(1) preparing a lysate from a tissue sample obtained from a subject,

(2) contacting the lysate with a compound of formula I

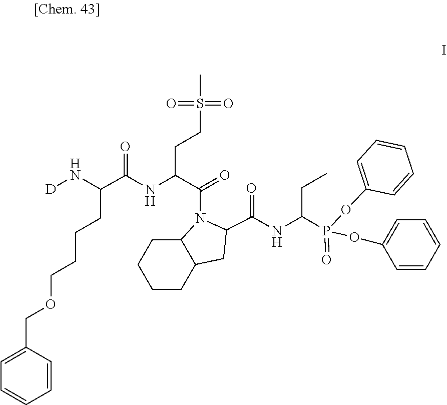

or a salt thereof,

wherein D is a detectable element.

44. The method of item 43, wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy.

45. The method of item 44, wherein the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

46. The method of any one of items 43 to 45, wherein the subject is a human subject.

47. The method of any one of the preceding items, wherein preparing the lysate comprises a clearing step.

48. The method of any one of items 1 to 42 and 47, wherein the gel electrophoresis is a one-dimensional or a two-dimensional gel electrophoresis.

49. The method of any one of items 1 to 42, 47, and 48, wherein the gel electrophoresis is an SDS-PAGE or a native PAGE, preferably an SDS-PAGE.

50. The method of any one of the preceding items, wherein the detectable element is selected from the group consisting of a fluorescent label, a biotin label, a radiolabel, a chelator, and a bioorthogonal ligation handle.

51. The method of any one of items 1 to 42, and 47 to 50, wherein the detectable signal is measured by fluorescence measurement or radiography.

52. The method of item 51, wherein the fluorescence measurement is in-gel fluorescence.

53. The method of item 51, wherein the fluorescence measurement is preceded by secondary labeling.

54. The method of item 53, wherein the secondary labeling is selected from the group consisting of secondary labeling with tagged streptavidin, secondary labeling with a fluorophore, and secondary labeling with a tagged antibody.

55. The method of any one of the preceding items, wherein in step (2), the lysate is contacted with a compound having the formula IA:

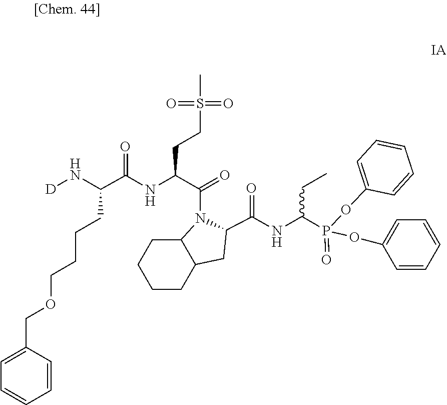

or a salt thereof,

wherein D is a detectable element.

56. The method of any one of items 1 to 55, wherein the detectable element is a fluorescent label.

57. The method of item 56, wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label.

58. The method of item 57, wherein the fluorescent label is a cyanine label.

59. The method of item 57, wherein the fluorescent label is a cyanine label having a formula selected from the following group of formulas:

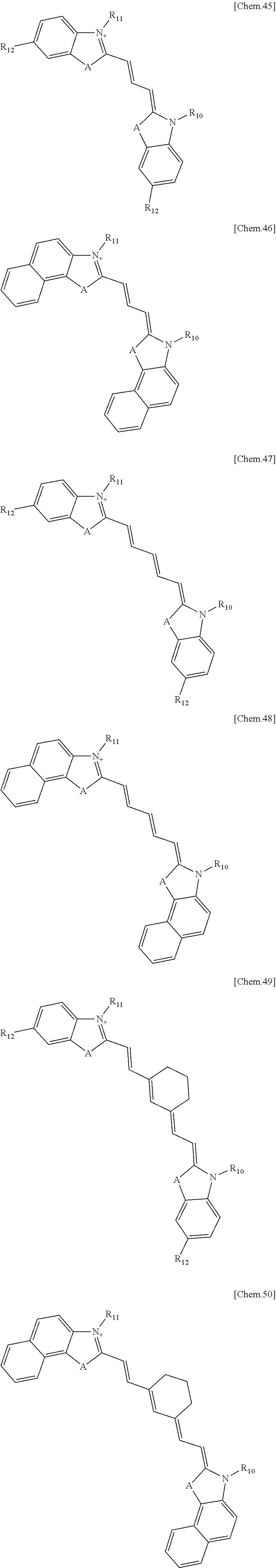

wherein in each of the above formulas,

A is selected from the group consisting of CH₂, C(CH₃)₂, C(C₂H₅)₂, NH, N(CH₃), N(C₂H₅), O, S, and Se;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, 6, 7, or 8;

q is 2, 3, 4, 5, 6, 7, or 8;

r is 2, 3, 4, 5, 6, 7, or 8;

$ represents the point of connection to the nitrogen atom of the cyanine moiety; and & represents the point of connection to the remainder of the molecule;

R₁₁ is selected from the group consisting of (C₁-C₈)alkyl, and (C₆-C₁₀)aryl; and

R₁₂ is H or a sulfo group.

60. The method of item 59, wherein

A is selected from the group consisting of CH₂, C(CH₃)₂, and C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is (C₁-C₈)alkyl; and

R₁₂ is H or a sulfo group.

61. The method of item 59, wherein

A is C(CH₃)₂ or C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is methyl, ethyl or propyl; and

R₁₂ is H or a sulfo group.

62. The method of item 59, wherein

A is C(CH₃)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 4, 5, or 6;

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H or a sulfo group.

63. The method of any one of items 59 to 62, wherein R₁₀ is $-(CH₂)ₚ—C(═O)-&.

64. The method of any one of items 59 to 63, wherein R₁₂ is a sulfo group.

65. The method of item 59, wherein

A is C(CH₃)₂;

R₁₀ is $-(CH₂)ₚ—C(═O)-&; wherein

p is 4, 5, or 6; and

$ represents the point of connection to the nitrogen atom of the cyanine moiety; and & represents the point of connection to the remainder of the molecule;

R₁₁ is methyl or ethyl; and

R₁₂ is a sulfo group.

66. The method of item 59, wherein

A is C(CH₃)₂;

R₁₀ is $-(CH₂)_{q}—C(═O)—NH—[CH₂CH₂O]ᵣ—CH₂CH₂—C(═O)-&;

wherein

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H.

67. The method of any one of items 59 to 66, wherein p is 5, q is 5 and r is 4.

68. The method of item 56, wherein the fluorescent label is a cyanine label having a formula selected from the following group of formulas:

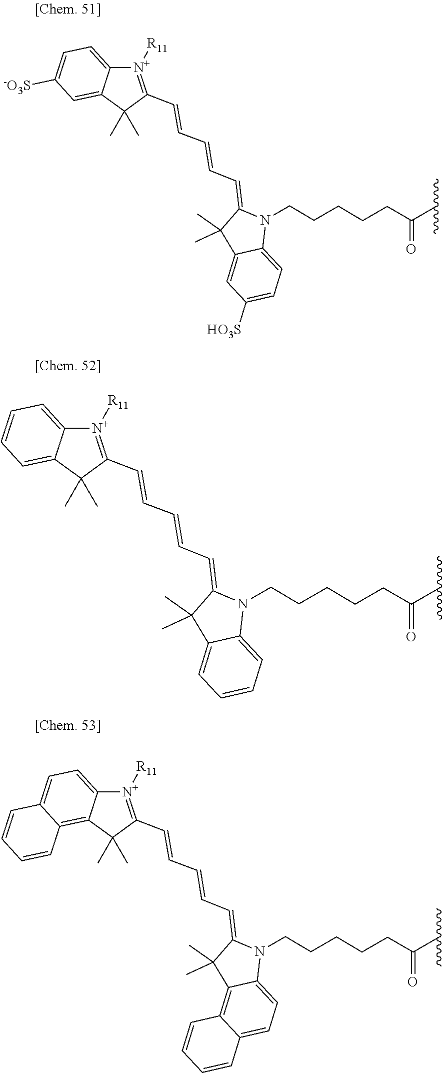

wherein in each of the above formulas,

the curled line represents the point of connection to the remainder of the molecule;

and R₁₁ is selected from the group consisting of (C_{F}C₈)alkyl, and (C₆-C₁₀)aryl.

69. The method of item 68, wherein R₁₁ is (C₁-C₈)alkyl.

70. The method of item 68, wherein R₁₁ is methyl or ethyl.

71. The method of item 56, wherein the fluorescent label is a cyanine label having the formula

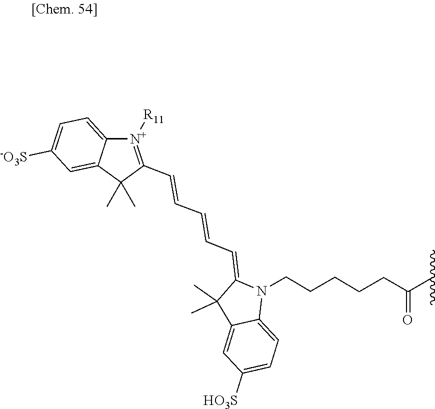

wherein the curled line represents the point of connection to the remainder of the molecule; and R₁₁ is methyl or ethyl.

72. The method of any one of items 1 to 65 and 67 to 71, wherein in step (2) the lysate is contacted with a compound of formula II

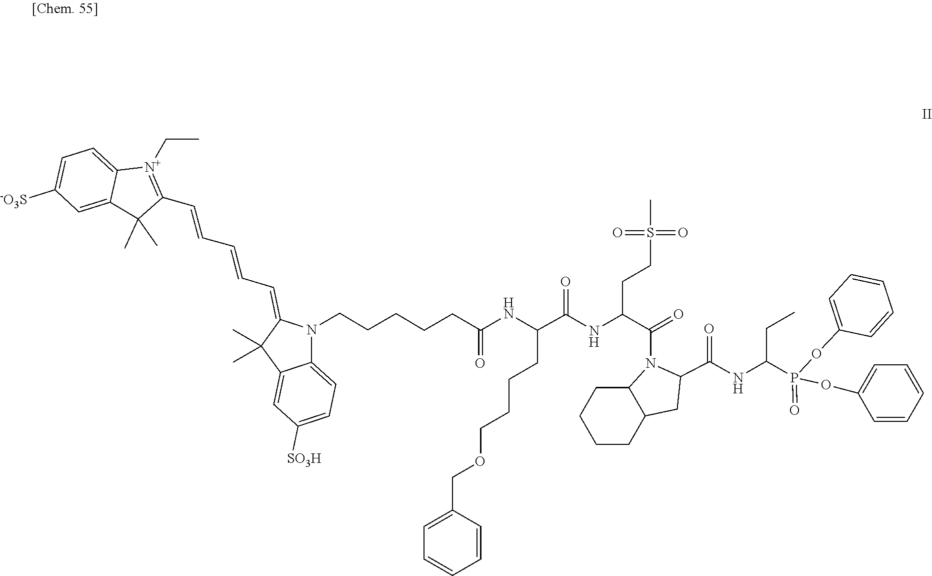

or a salt thereof.

73. The method of any one of items 1 to 65 and 67 to 72, wherein in step (2) the lysate is contacted with a compound of formula IIA

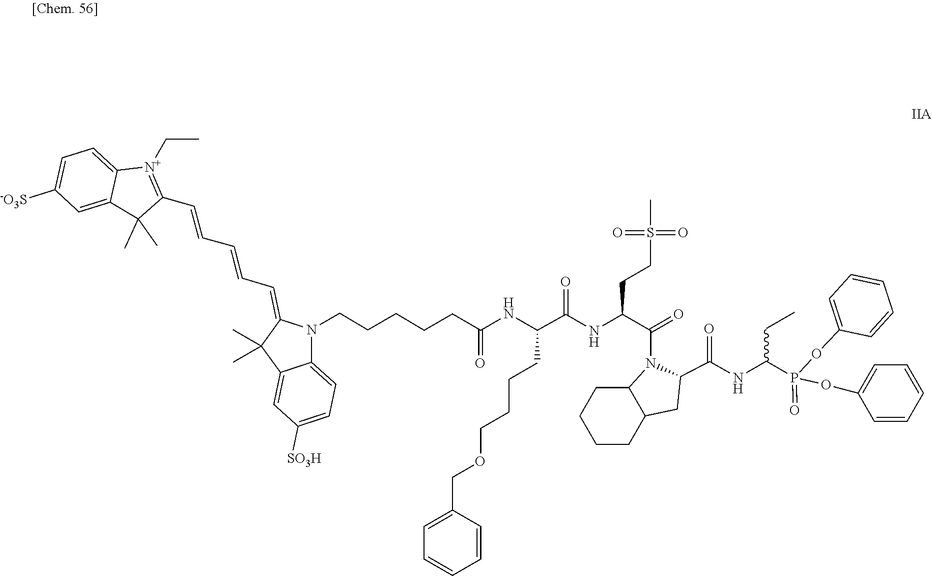

or a salt thereof.

74. The method of any one of items 56 to 73, wherein the detectable signal is measured by in-gel fluorescence.

75. An in vitro method of diagnosing an inflammatory bowel disease in a subject, comprising detecting an activated form of NE that is a trimmed form of mature NE.

76. The method of item 75, wherein the subject is a human subject.

77. The method of item 75 or 76, wherein the method comprises a step of contacting the activated form of NE with an activity-based probe.

78. The method of any one of items 75 to 77, wherein the method comprises a step of contacting the activated form of NE with an anti-NE-antibody.

79. A compound of formula I

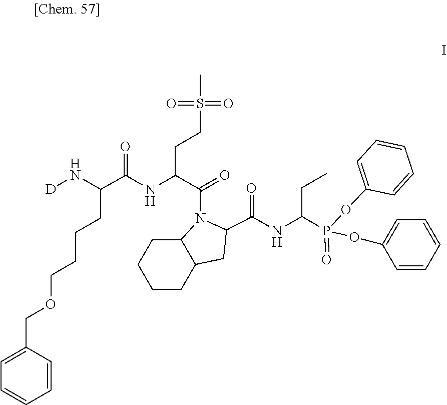

or a salt thereof,

wherein D is a detectable element,

with the proviso that compounds wherein D corresponds to one of the following formulas are excluded:

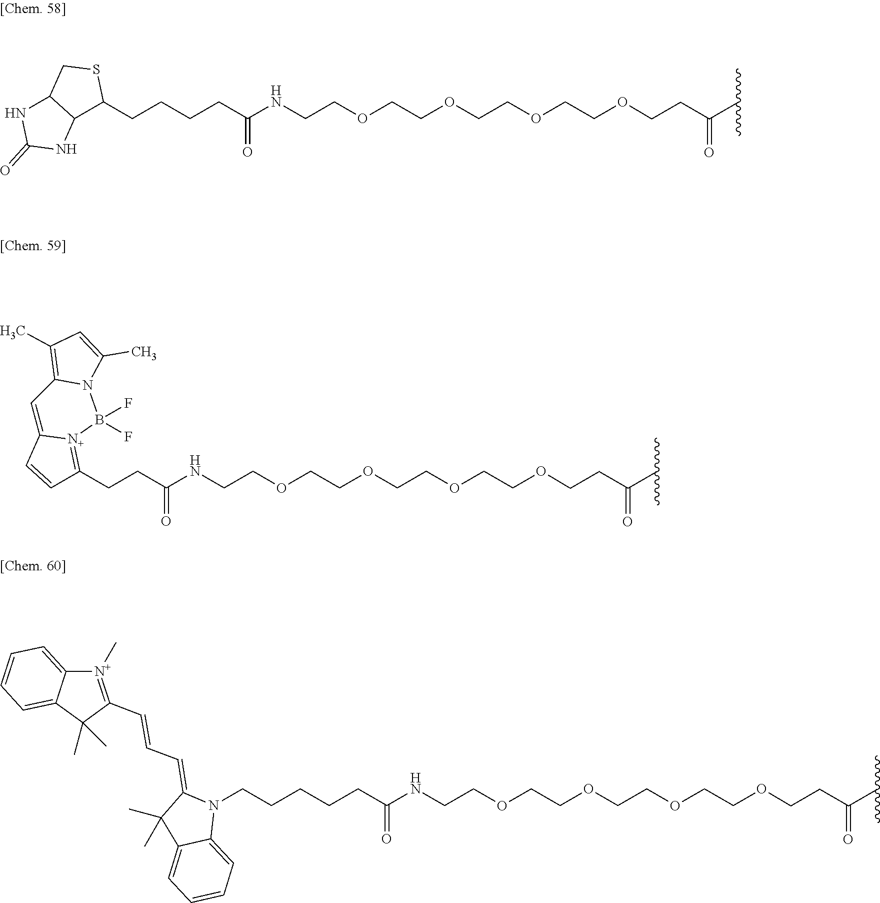

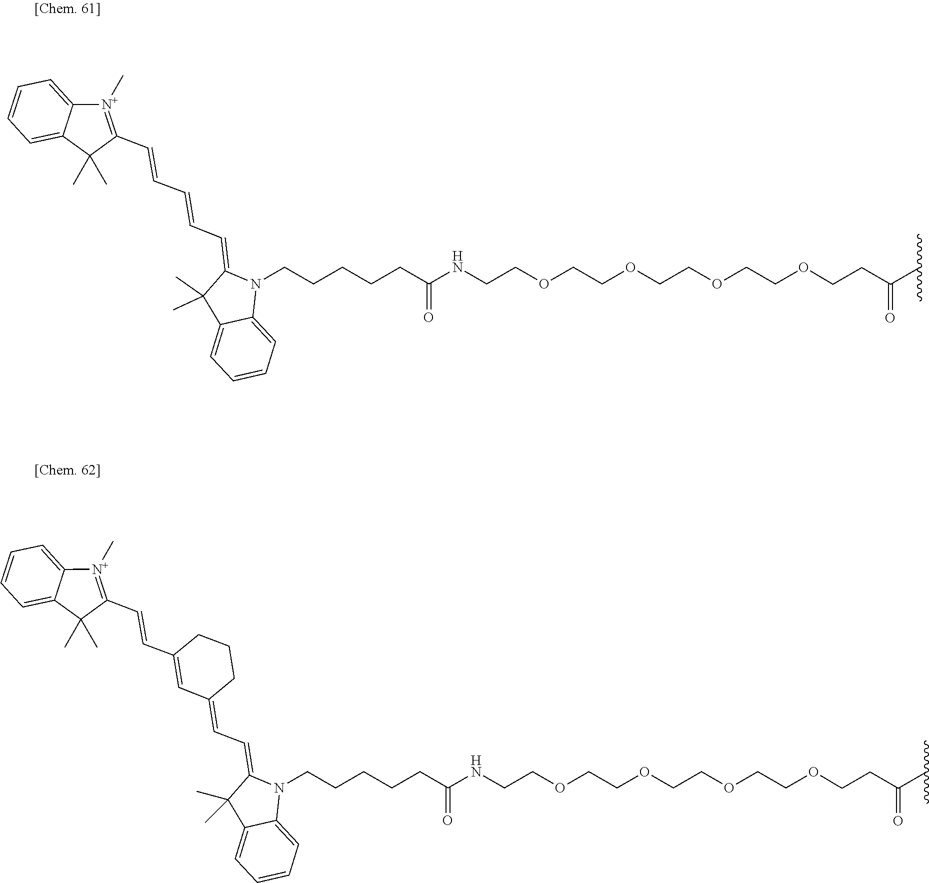 [SMILES: *C(=O)CCOCCOCCOCCOCCNC(=O)CCCCCN1/C(=C/C=C/C=C/C2=[N+](C)c3ccccc3C2(C)C)C(C)(C)c2ccccc21 ; *C(=O)CCOCCOCCOCCOCCNC(=O)CCCCCN1/C(=C/C=C2C=C(/C=C/C3=[N+](C)c4ccccc4C3(C)C)CCC/2)C(C)(C)c2ccccc21]

wherein in each of the above formulas, the curled line represents the point of connection to the remainder of the molecule.

80. The compound of item 79 having the formula IA:

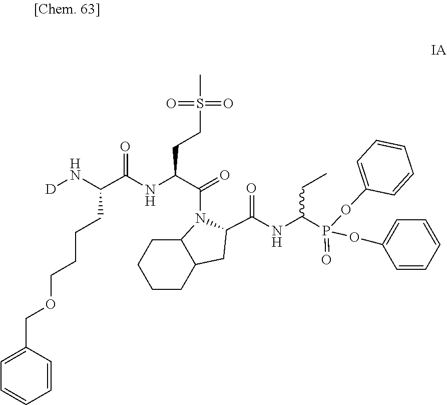

or a salt thereof,

wherein D is a detectable element.

81. The compound of item 79 or 80, wherein the detectable element is selected from the group consisting of a fluorescent label, a biotin label, a radiolabel, a chelator, and a bioorthogonal ligation handle.

82. The compound of any one of items 79 to 81, wherein the detectable element is a fluorescent label.

83. The compound of item 82, wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label.

84. The compound of item 82, wherein the fluorescent label is a cyanine label.

85. The compound of item 82, wherein the fluorescent label is a cyanine label having a formula selected from the following group of formulas:

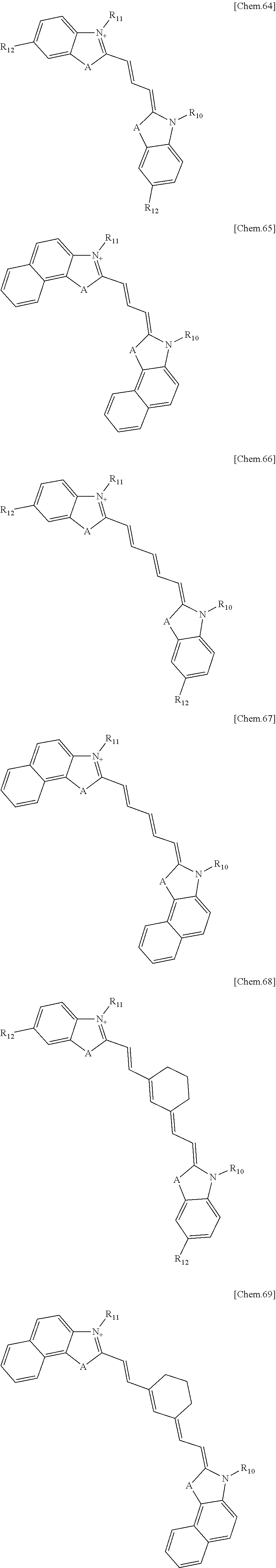

wherein in each of the above formulas,

A is selected from the group consisting of CH₂, C(CH₃)₂, C(C₂H₅)₂, NH, N(CH₃), N(C₂H₅), O, S, and Se;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, 6, 7, or 8;

q is 2, 3, 4, 5, 6, 7, or 8;

r is 2, 3, 4, 5, 6, 7, or 8;

$ represents the point of connection to the nitrogen atom of the cyanine moiety; and & represents the point of connection to the remainder of the molecule;

R₁₁ is selected from the group consisting of (C₁-C₈)alkyl, and (C₆-C₁₀)aryl; and

R₁₂ is H or a sulfo group.

86. The compound of item 85, wherein

A is selected from the group consisting of CH₂, C(CH₃)₂, and C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is (C₁-C₈)alkyl; and

R₁₂ is H or a sulfo group.

87. The compound of item 85, wherein

A is C(CH₃)₂ or C(C₂H₅)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 2, 3, 4, 5, or 6;

q is 2, 3, 4, 5, or 6;

r is 2, 3, 4, 5, or 6;

$ represents the point of connection to the nitrogen atom of the cyanine moiety; and & represents the point of connection to the remainder of the molecule;

R₁₁ is methyl, ethyl or propyl; and

R₁₂ is H or a sulfo group.

88. The compound of item 85, wherein

A is C(CH₃)₂;

R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;

wherein

p is 4, 5, or 6;

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H or a sulfo group.

89. The compound of any one of items 85 to 88, wherein R₁₀ is $-(CH₂)ₚ—C(═O)-&.

90. The compound of any one of items 85 to 89, wherein R₁₂ is a sulfo group.

91. The compound of item 85, wherein

A is C(CH₃)₂;

R₁₀ is $-(CH₂)ₚ—C(═O)-&; wherein

p is 4, 5, or 6; and

$ represents the point of connection to the nitrogen atom of the cyanine moiety; and & represents the point of connection to the remainder of the molecule;

R₁₁ is methyl or ethyl; and

R₁₂ is a sulfo group.

92. The compound of item 85, wherein

A is C(CH₃)₂;

R₁₀ is $-(CH₂)_{q}—C(═O)—NH—[CH₂CH₂O]ᵣ—CH₂CH₂—C(═O)-&;

wherein

q is 4, 5, or 6;

r is 3, 4, 5, or 6;

$ represents the point of connection to the nitrogen atom of the cyanine moiety; and & represents the point of connection to the remainder of the molecule;

R₁₁ is methyl or ethyl; and

R₁₂ is H.

93. The compound of any one of items 85 to 92, wherein p is 5, q is 5 and r is 4.

94. The compound of item 82, wherein the fluorescent label is a cyanine label having a formula selected from the following group of formulas:

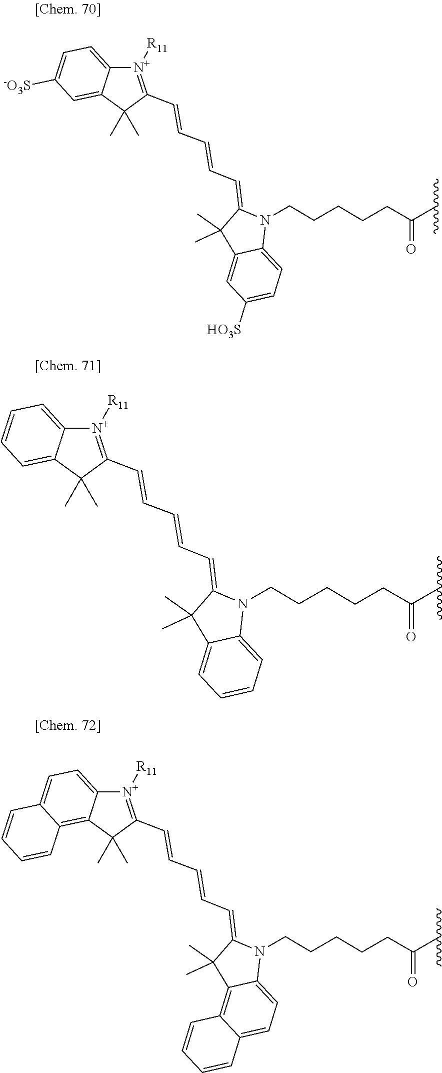

wherein in each of the above formulas,

the curled line represents the point of connection to the remainder of the molecule;

and R₁₁ is selected from the group consisting of (C₁-C₈)alkyl, and (C₆-C₁₀)aryl.

95. The compound of item 94, wherein R₁₁ is (C_{F}C₈)alkyl.

96. The compound of item 94, wherein R₁₁ is methyl or ethyl.

97. The compound of item 82, wherein the fluorescent label is a cyanine label having the formula

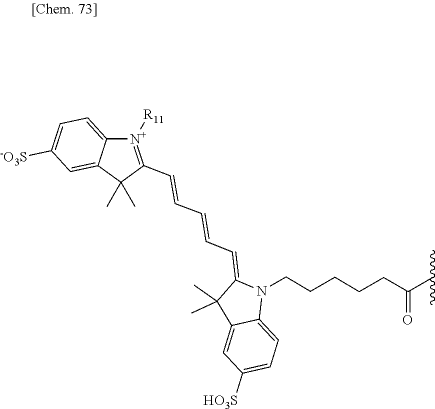

wherein the curled line represents the point of connection to the remainder of the molecule; and R₁₁ is methyl or ethyl.

98. A compound of formula II

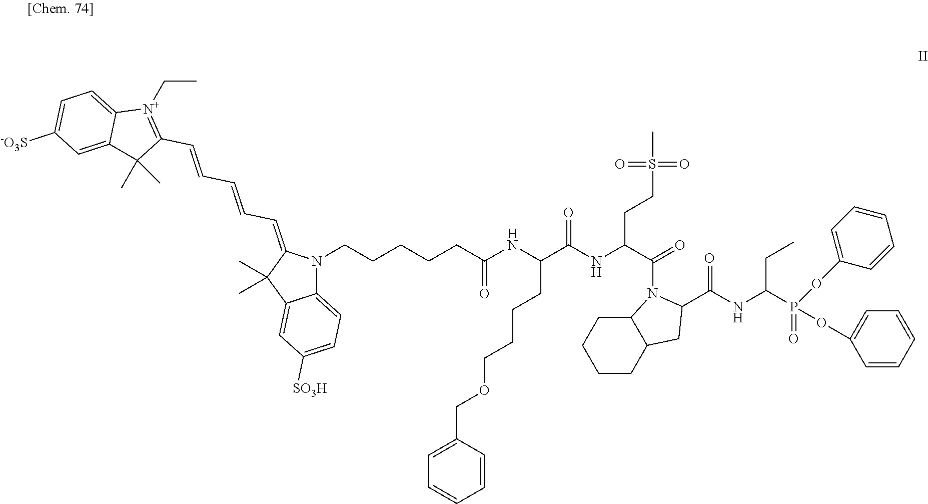

or a salt thereof.

99. A compound of formula IIA

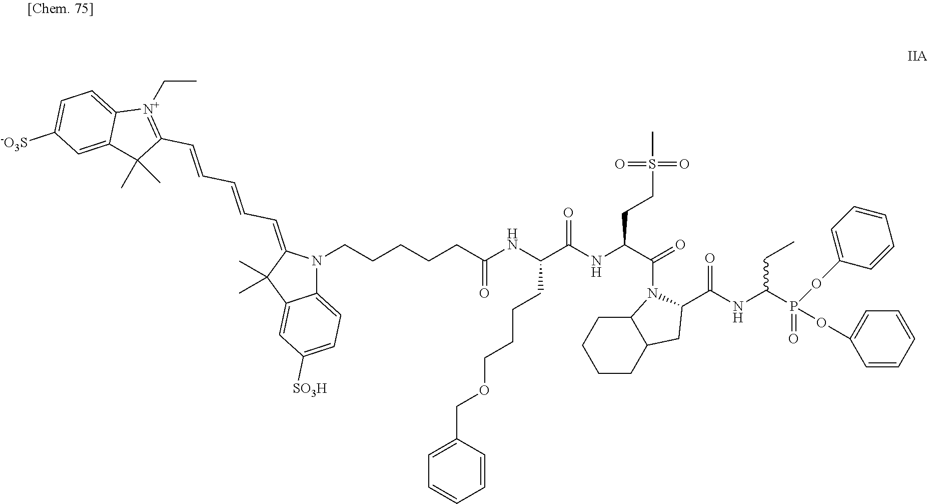

or a salt thereof.

100. A composition comprising a compound of any one of items 79 to 99 or a salt thereof, and an excipient.

1. A method of detecting neutrophil elastase (NE) activity in a tissue sample lysate, comprising
(1) preparing the lysate from a tissue sample obtained from a subject,
(2) contacting the lysate with a compound of formula I 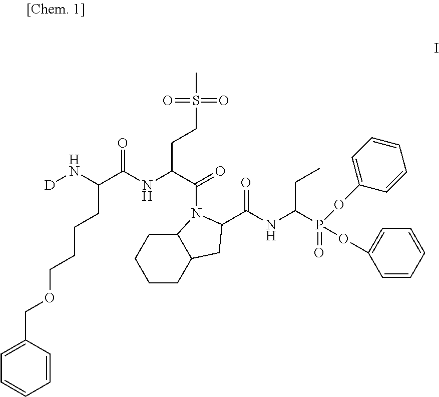
or a salt thereof,
wherein D is a detectable element,
(3) subsequently subjecting at least an aliquot of the lysate of step (2) to gel electrophoresis; and thereafter
(4) measuring a detectable signal.

2. The method of claim 1, further comprising after step (3) a step (5) immunoblotting with an anti-NE antibody, and/or
wherein additionally the following steps are performed:
(3a) immunoprecipitating the compound of formula I in a separate aliquot of the lysate of step (2) using an antibody specific for the compound of formula I or a part thereof,
(4a) subsequently analyzing co-precipitated material, optionally wherein the analysis of step (4a) comprises
gel electrophoresis and subsequent immunoblot using an anti-NE antibody, or
protein sequencing,
and preferably comprises gel electrophoresis and subsequent immunoblot using an anti-NE antibody.

3. The method of claim 1 or 2,
wherein prior to step (2), an aliquot of the lysate of step (1) is pretreated with a specific NE inhibitor, and wherein the pretreated aliquot is subsequently processed analogously to the not pretreated lysate of step (1); and/or
wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy, optionally wherein the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

4. The method of any one of the preceding claims, wherein the subject is a human subject.

5. The method of any one of the preceding claims, wherein an activated form of NE that is a trimmed form of mature NE is detected, optionally wherein the tissue sample is selected from the group consisting of an oral biopsy, an esophagus sample, a stomach sample, a small intestine sample, a colon sample, a proximal colon sample, a distal colon sample, a rectal sample, a fecal sample, and a mucosal biopsy, optionally wherein the mucosal biopsy is selected from the group consisting of an oral mucosal biopsy, an esophagus mucosal biopsy, a small intestine mucosal biopsy, a colon mucosal biopsy, and a rectal mucosal biopsy.

6. A method of diagnosing a disease associated with NE activity in a subject comprising
(1) preparing a lysate from a tissue sample obtained from the subject,
(2) contacting the lysate with a compound of formula I 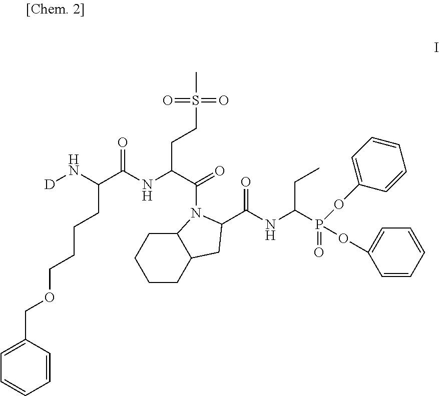
or a salt thereof,
wherein D is a detectable element,
(3) subsequently subjecting the lysate to gel electrophoresis; and thereafter
(4) measuring a detectable signal.

7. The method of claim 6,
wherein the disease associated with NE activity is selected from the group consisting of a celiac disease, a gastrointestinal motility disorder, pain, itch, a skin disorder, diet-induced obesity, a metabolic disorder, asthma, rheumatoid arthritis, periodontitis, an inflammatory GI disorder, a functional GI disorder, a cancer, a fibrotic disease, metabolic dysfunction, a neurological disease, a chronic obstructive pulmonary disease (COPD), and an infection; or
wherein the disease associated with NE activity is selected from the group consisting of an inflammatory bowel disease, an infection, a chronic obstructive pulmonary disease, and a cancer.

8. The method of claim 6 or 7, further comprising after step (3) a step (5) immunoblotting with an anti-NE antibody; and/or
wherein additionally the following steps are performed:
(3a) immunoprecipitating the compound of formula I in a separate aliquot of the lysate of step (2) using an antibody specific for the compound of formula I or a part thereof,
(4a) subsequently analyzing co-precipitated material, optionally wherein the analysis of step (4a) comprises
gel electrophoresis and subsequent immunoblot using an anti-NE antibody, or
protein sequencing,
and preferably comprises gel electrophoresis and subsequent immunoblot using an anti-NE antibody.

9. The method of any one of claims 6 to 8,
wherein prior to step (2), an aliquot of the lysate of step (1) is pretreated with a specific NE inhibitor, and wherein the pretreated aliquot is subsequently processed analogously to the not pretreated lysate of step (1); and/or
wherein the tissue sample is selected from the group consisting of an oral biopsy, an esophagus sample, a stomach sample, a small intestine sample, a lung sample, a sputum sample, a pancreas sample, a bone marrow sample, a colon sample, a distal colon sample, a proximal colon sample, a breast biopsy, a prostate biopsy, a rectal biopsy, a liver sample, a skin sample, a tumor sample, a fecal sample, and a mucosal biopsy, optionally wherein the mucosal biopsy is selected from the group consisting of a colon mucosal biopsy, a distal colon mucosal biopsy, a proximal colon mucosal biopsy, a small intestine mucosal biopsy, a lung mucosal biopsy, a rectal mucosal biopsy, an esophagus mucosal biopsy, and an oral mucosal biopsy.

10. The method of any one of claims 6 to 9, wherein the subject is a human subject.

11. The method of any one of claims 6 to 10, wherein the method is for diagnosing an inflammatory bowel disease,
optionally wherein an activated form of NE that is a trimmed form of mature NE is detected, optionally wherein the subject is diagnosed as having an inflammatory bowel disease if the activated form of NE is detected.

12. The method of claim 11, wherein the tissue sample is selected from the group consisting of an oral biopsy, an esophagus sample, a stomach sample, a small intestine sample, a colon sample, a proximal colon sample, a distal colon sample, a rectal sample, a fecal sample, and a mucosal biopsy, optionally wherein the tissue sample is a mucosal biopsy selected from the group consisting of an oral mucosal biopsy, an esophagus mucosal biopsy, a small intestine mucosal biopsy, a colon mucosal biopsy, and a rectal mucosal biopsy.

13. The method of any one of claims 6 to 12, wherein the inflammatory bowel disease is selected from the group consisting of acute colitis, ulcerative colitis, Crohn's disease, microscopic colitis, diversion colitis, Behcet's disease, immuno-oncology colitis, chemotherapy/radiation colitis, Graft versus Host Disease colitis, collagenous colitis, lymphocytic colitis, and indeterminate colitis and pouchitis.

14. The method of any one of claims 6 to 13, wherein the inflammatory bowel disease is ulcerative colitis; or wherein the inflammatory bowel disease is Crohn's disease.

15. The method of any one of claims 6 to 10, wherein the method is for diagnosing an infection,
optionally wherein the infection is selected from the group consisting of a bacterial infection and a fungal infection; and/or
optionally wherein the tissue sample is a sample from an infected tissue.

16. The method of claim 15, wherein the infection is an infection of the lung, optionally wherein the infection of the lung is a bacterial infection, optionally wherein the bacterial infection is an infection with Legionella.

17. The method of claim 16, wherein the tissue sample is selected from the group consisting of a lung sample, a lung mucosal biopsy or a sputum sample.

18. The method of any one of claims 6 to 10, wherein the method is for diagnosing a cancer,
optionally wherein the tissue sample is selected from the group consisting of a tumor sample, an oral biopsy, an oral mucosal biopsy, a breast biopsy, a prostate biopsy, a colon biopsy, a colon mucosal biopsy, a rectal biopsy, a rectal mucosal biopsy, a lung biopsy, a lung mucosal biopsy, and a sputum sample; and/or
optionally wherein the cancer is selected from the group consisting of an oral cancer, a breast cancer, a prostate cancer, a colorectal cancer, and a lung cancer.

19. The method of claim 18, wherein the cancer is an oral cancer and the oral cancer is a squamous cell carcinoma.

20. The method of any one of claims 6 to 10, wherein the method is for diagnosing a chronic obstructive pulmonary disease, optionally wherein the tissue sample is selected from the group consisting of a lung sample, a lung mucosal biopsy, and a sputum sample.

21. The method of any one of the preceding claims, wherein preparing the lysate comprises a clearing step.

22. The method of any one of the preceding claims, wherein the gel electrophoresis is a one-dimensional or a two-dimensional gel electrophoresis, and/or wherein the gel electrophoresis is an SDS-PAGE or a native PAGE, preferably an SDS-PAGE.

23. The method of any one of the preceding claims, wherein the detectable element is selected from the group consisting of a fluorescent label, a biotin label, a radiolabel, a chelator, and a bioorthogonal ligation handle; and/or
wherein the detectable signal is measured by fluorescence measurement or radiography,
optionally wherein the fluorescence measurement is in-gel fluorescence; or
optionally wherein the fluorescence measurement is preceded by secondary labeling, optionally wherein the secondary labeling is selected from the group consisting of secondary labeling with tagged streptavidin, secondary labeling with a fluorophore, and secondary labeling with a tagged antibody.

24. The method of any one of the preceding claims, wherein in step (2), the lysate is contacted with a compound having the formula IA: 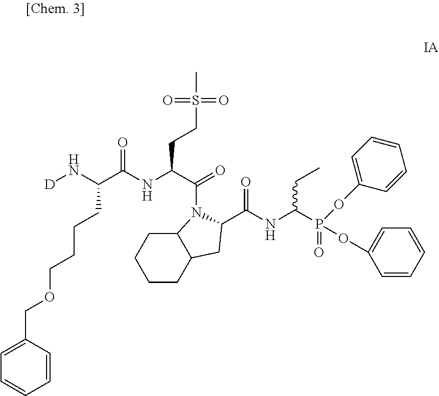
or a salt thereof,
wherein D is a detectable element.

25. The method of any one of claims 1 to 24, wherein the detectable element is a fluorescent label, optionally
wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label, or
wherein the fluorescent label is a cyanine label.

26. The method of claim 25, wherein the fluorescent label is a cyanine label having a formula selected from the following group of formulas: 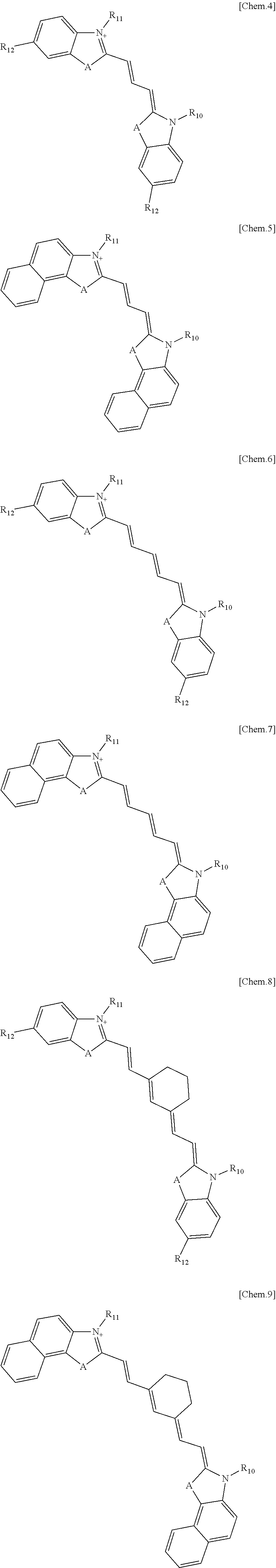
wherein in each of the above formulas,
A is selected from the group consisting of CH₂, C(CH₃)₂, C(C₂H₅)₂, NH, N(CH₃), N(C₂H₅), O, S, and Se;
R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;
wherein
p is 2, 3, 4, 5, 6, 7, or 8;
q is 2, 3, 4, 5, 6, 7, or 8;
r is 2, 3, 4, 5, 6, 7, or 8;
$ represents the point of connection to the nitrogen atom of the cyanine moiety; and & represents the point of connection to the remainder of the molecule;
R₁₁ is selected from the group consisting of (C₁-C₈)alkyl, and (C₆-C₁₀) aryl; and
R₁₂ is H or a sulfo group.

27. The method of claim 25, wherein the fluorescent label is a cyanine label having a formula selected from the following group of formulas: 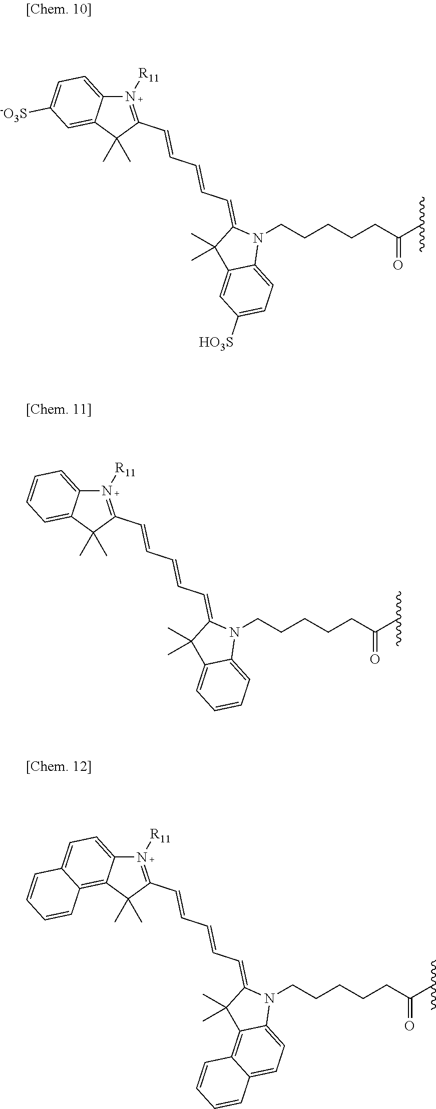
wherein in each of the above formulas,
the curled line represents the point of connection to the remainder of the molecule;
and R₁₁ is selected from the group consisting of (C₁-C₈)alkyl, and (C₆-C₁₀)aryl; or
wherein the fluorescent label is a cyanine label having the formula 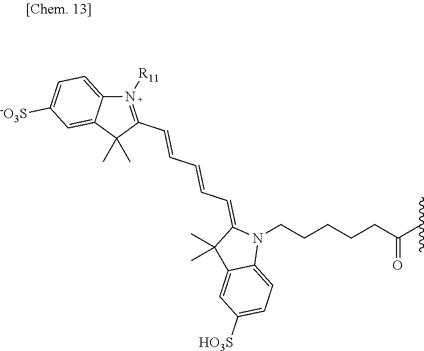
wherein the curled line represents the point of connection to the remainder of the molecule; and R₁₁ is methyl or ethyl.

28. The method of any one of claims 1 to 27,
wherein in step (2) the lysate is contacted with a compound of formula II 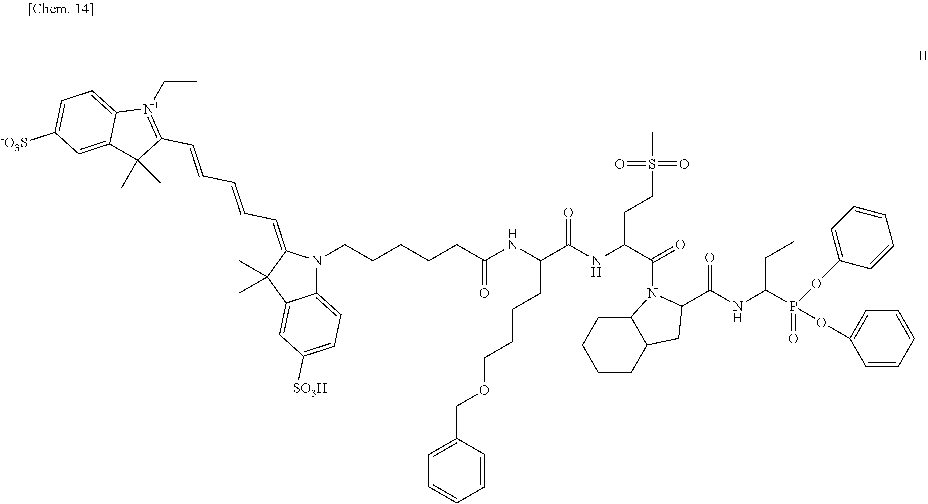
or a salt thereof; or
wherein in step (2) the lysate is contacted with a compound of formula IIA 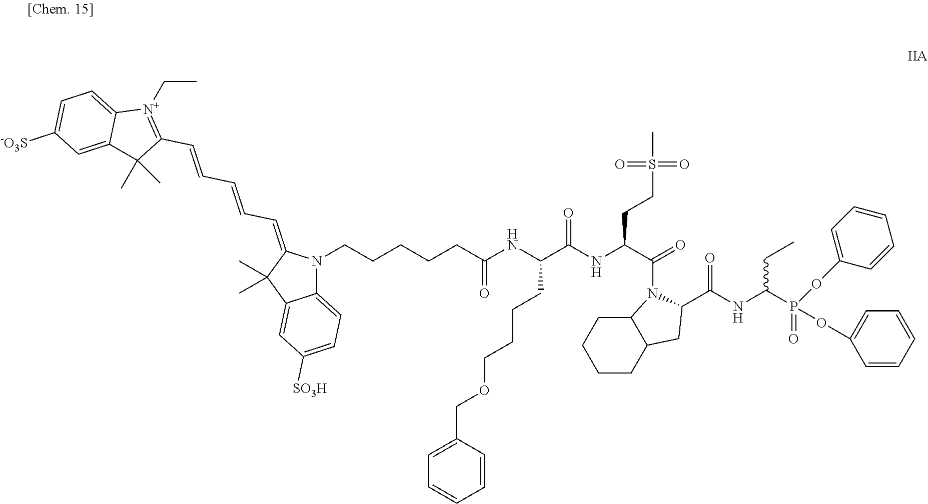
or a salt thereof.

29. The method of any one of claims 25 to 28, wherein the detectable signal is measured by in-gel fluorescence.

30. An in vitro method of diagnosing an inflammatory bowel disease in a subject, comprising detecting an activated form of NE that is a trimmed form of mature NE, optionally wherein the subject is a human subject.

31. The method of claim 30, wherein the method comprises a step of contacting the activated form of NE with an activity-based probe; and/or
wherein the method comprises a step of contacting the activated form of NE with an anti-NE-antibody.

32. A compound of formula I 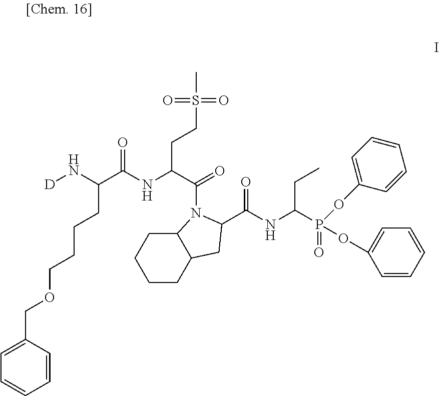
or a salt thereof,
wherein D is a detectable element,
with the proviso that compounds wherein D corresponds to one of the following formulas are excluded: 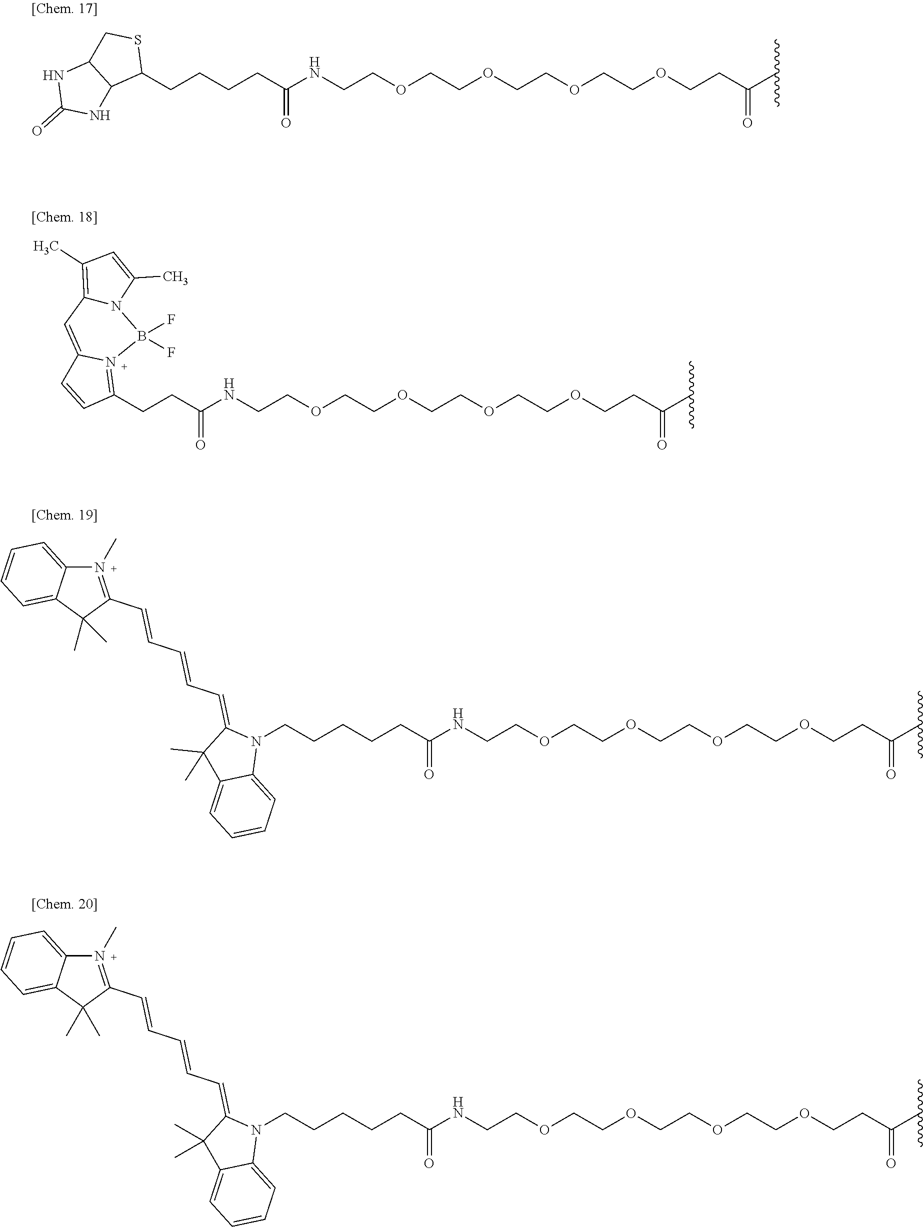 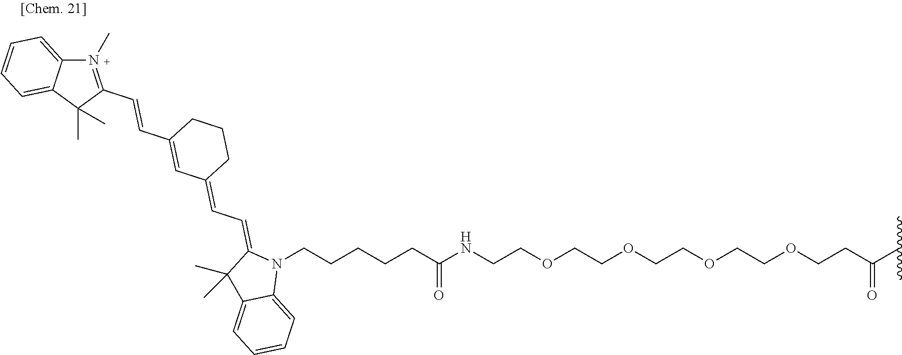
wherein in each of the above formulas, the curled line represents the point of connection to the remainder of the molecule.

33. The compound of claim 32 having the formula IA: 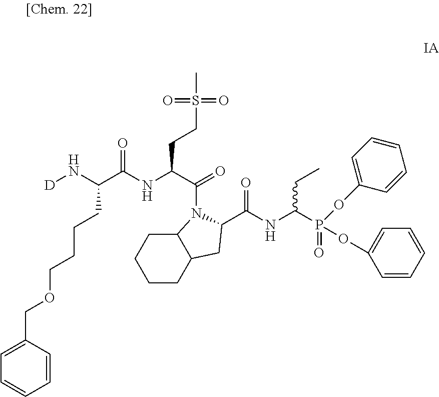
or a salt thereof,
wherein D is a detectable element.

34. The compound of claim 32 or 33, wherein the detectable element is selected from the group consisting of a fluorescent label, a biotin label, a radiolabel, a chelator, and a bioorthogonal ligation handle.

35. The compound of any one of claims 32 to 34, wherein the detectable element is a fluorescent label, optionally
wherein the fluorescent label is selected from the group consisting of a fluorescein, an Oregon green, a bora-diaza-indecene dye, a rhodamine dye, a benzopyrillium dye, a coumarin dye, a cyanine label or a benzoindole label, or
wherein the fluorescent label is a cyanine label.

36. The compound of claim 35, wherein the fluorescent label is a cyanine label having a formula selected from the following group of formulas: 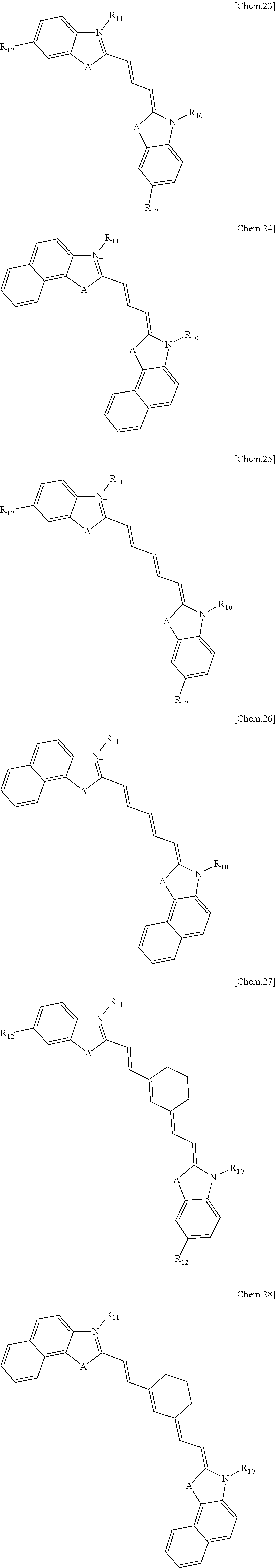
wherein in each of the above formulas,
A is selected from the group consisting of CH₂, C(CH₃)₂, C(C₂H₅)₂, NH, N(CH₃), N(C₂H₅), O, S, and Se;
R₁₀ is selected from the group consisting of $-(CH₂)ₚ—C(═O)-& and $-(CH₂)_{q}—C(═O)—NH—[CH₂CH₂O]ᵣ—CH₂CH₂—C(═O)-&;
wherein
p is 2, 3, 4, 5, 6, 7, or 8;
q is 2, 3, 4, 5, 6, 7, or 8;
r is 2, 3, 4, 5, 6, 7, or 8;
$ represents the point of connection to the nitrogen atom of the cyanine moiety; and & represents the point of connection to the remainder of the molecule;
R₁₁ is selected from the group consisting of (C₁-C₈)alkyl, and (C₆-C₁₀) aryl; and
R₁₂ is H or a sulfo group.

37. The compound of claim 35, wherein the fluorescent label is a cyanine label having a formula selected from the following group of formulas: 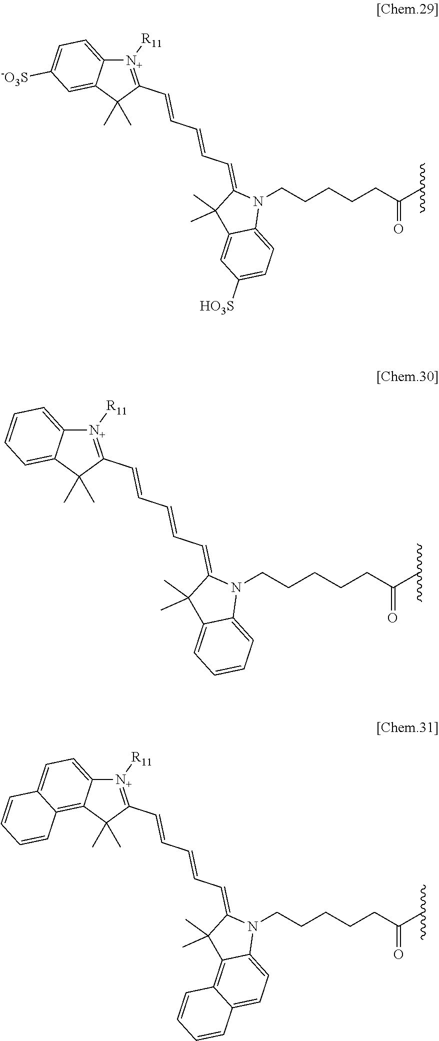
wherein in each of the above formulas,
the curled line represents the point of connection to the remainder of the molecule;
and R₁₁ is selected from the group consisting of (C₁-C₈)alkyl, and (C₆-C₁₀)aryl; or
wherein the fluorescent label is a cyanine label having the formula 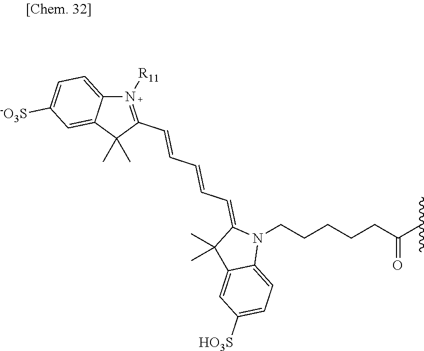
wherein the curled line represents the point of connection to the remainder of the molecule; and R₁₁ is methyl or ethyl.

38. A compound of formula II 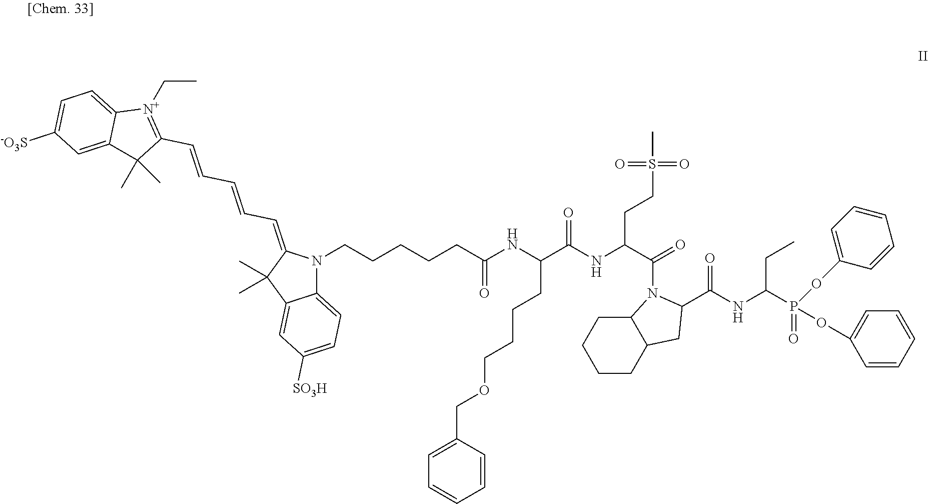
or a salt thereof.

39. A compound of formula IIA 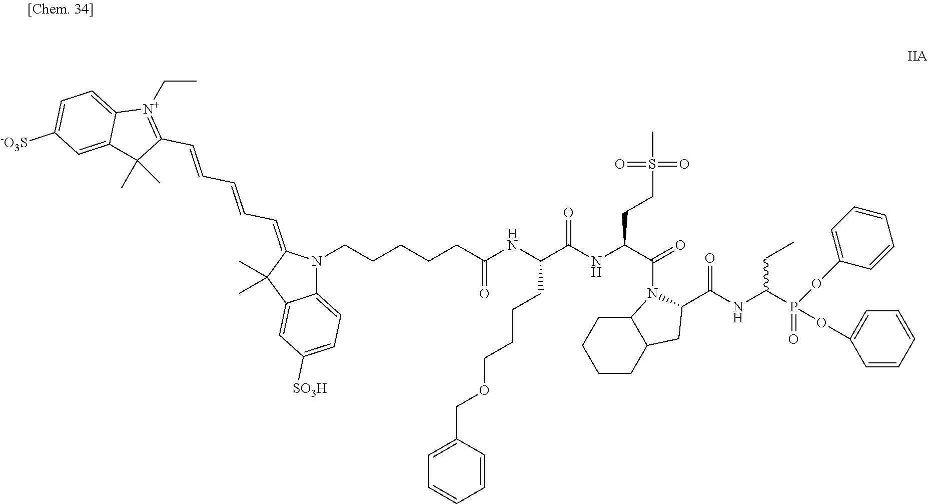
or a salt thereof.

40. A composition comprising a compound of any one of claims 32 to 39
or a salt thereof, and an excipient.
